SEQUENCE LISTING (47 sequences)

SEQ ID NO: 1 - DNA, 1707 bp, Sendai virus
  source 1..1707: mol_type = other DNA
atggatcaagatgccttcattcttaaagaagattctgaagttgagagggaggcgccaggaggaagagagtcgctctcggatgttatcggattcctcgatgctgtcctgtcgagtgaaccaactgacatcggaggggacagaagctggctccacaacaccatcaacactccccaaggaccaggctctgcccatagagccaaaagtgagggcgaaggagaagtctcaacaccgtcgacccaagataatcgatcaggtgaggagagtagagtctctgggagaacaagcaagccagaggcagaagcacatgctggaaaccttgataaacaaaatatacaccgggcctttgggggaagaactggtacaaactctgtatctcaggatctgggcgatggaggagactccggaatccttgaaaatcccccaaatgagagaggatatccgagatcaggtattgaagatgaaaacagagagatggctgcgcaccctgataagaggggagaagaccaagctgaaggacttccagaagaggtacgaggaggtacatccctacctgatgaaggagaaggtggagcaagtaataatggaagaagcatggagcctggcagctcacatagtgcaagagtaactggggtcctggtgattcctagccccgaactcgaagaggctgtgctacggaggaacaaaagaagacctaccaacagtgggtccaaacctcttactccagcaaccgtgcctggcacccggtccccaccgctgaatcgttacaacagcacagggtcaccaccaggaaaacccccatctacacaggatgagcacatcaactctggggacacccccgccgtcagggtcaaagaccggaaaccaccaatagggacccgctctgtctcagattgtccagccaacggccgcccaatccacccgggtctagagaccgactcaacaaaaaagggcataggagagaacacatcatctatgaaagagatggctacattgttgacgagtcttggtgtaatccagtctgctcaagaattcgagtcatcccgagacgcgagttatgtgtttgcaagacgtgccctaaagtctgcaaactatgcagagatgacattcaatgtatgcggcctgatcctttctgccgagaaatcttccgctcgtaaggtagatgagaacaaacaactgctcaaacagatccaagagagcgtggaatcattccgggatatttacaagagattctctgagtatcagaaagaacagaactcattgctgatgtccaacctatctacacttcatatcatcacagatagaggtggcaagactgacaacacagactcccttacaaggtccccctccgtttttgcaaaatcaaaagagaacaagactaaggctaccaggtttgacccatctatggagaccctagaagatatgaagtacaaaccggacctaatccgagaggatgaatttagagatgagatccgcaacccgttgtaccaagagagggacacagaacccagggcctcaaacgcatcacgcctcctcccctccaaagagaagcccacaatgcactctctcaggctcgtcatagagagcagtcccctaagcagagctgagaaagcagcatatgtgaaatcattatccaagtgcaagacagaccaagaggttaaggcagtcatggaactcgtagaagaggacatagagtcactgaccaactag

SEQ ID NO: 2 - protein, 568 aa, Sendai virus
MDQDAFILKEDSEVEREAPGGRESLSDVIGFLDAVLSSEPTDIGGDRSWLHNTINTPQGPGSAHRAKSEGEGEVSTPSTQDNRSGEESRVSGRTSKPEAEAHAGNLDKQNIHRAFGGRTGTNSVSQDLGDGGDSGILENPPNERGYPRSGIEDENREMAAHPDKRGEDQAEGLPEEVRGGTSLPDEGEGGASNNGRSMEPGSSHSARVTGVLVIPSPELEEAVLRRNKRRPTNSGSKPLTPATVPGTRSPPLNRYNSTGSPPGKPPSTQDEHINSGDTPAVRVKDRKPPIGTRSVSDCPANGRPIHPGLETDSTKKGIGENTSSMKEMATLLTSLGVIQSAQEFESSRDASYVFARRALKSANYAEMTFNVCGLILSAEKSSARKVDENKQLLKQIQESVESFRDIYKRFSEYQKEQNSLLMSNLSTLHIITDRGGKTDNTDSLTRSPSVFAKSKENKTKATRFDPSMETLEDMKYKPDLIREDEFRDEIRNPLYQERDTEPRASNASRLLPSKEKPTMHSLRLVIESSPLSRAEKAAYVKSLSKCKTDQEVKAVMELVEEDIESLTN

SEQ ID NO: 3 - DNA, 1479 bp, Sendai virus
  source 1..1479: mol_type = other DNA
atgtcgacccaagataatcgatcaggtgaggagagtagagtctctgggagaacaagcaagccagaggcagaagcacatgctggaaaccttgataaacaaaatatacaccgggcctttgggggaagaactggtacaaactctgtatctcaggatctgggcgatggaggagactccggaatccttgaaaatcccccaaatgagagaggatatccgagatcaggtattgaagatgaaaacagagagatggctgcgcaccctgataagaggggagaagaccaagctgaaggacttccagaagaggtacgaggaggtacatccctacctgatgaaggagaaggtggagcaagtaataatggaagaagcatggagcctggcagctcacatagtgcaagagtaactggggtcctggtgattcctagccccgaactcgaagaggctgtgctacggaggaacaaaagaagacctaccaacagtgggtccaaacctcttactccagcaaccgtgcctggcacccggtccccaccgctgaatcgttacaacagcacagggtcaccaccaggaaaacccccatctacacaggatgagcacatcaactctggggacacccccgccgtcagggtcaaagaccggaaaccaccaatagggacccgctctgtctcagattgtccagccaacggccgcccaatccacccgggtctagagaccgactcaacaaaaaagggcataggagagaacacatcatctatgaaagagatggctacattgttgacgagtcttggtgtaatccagtctgctcaagaattcgagtcatcccgagacgcgagttatgtgtttgcaagacgtgccctaaagtctgcaaactatgcagagatgacattcaatgtatgcggcctgatcctttctgccgagaaatcttccgctcgtaaggtagatgagaacaaacaactgctcaaacagatccaagagagcgtggaatcattccgggatatttacaagagattctctgagtatcagaaagaacagaactcattgctgatgtccaacctatctacacttcatatcatcacagatagaggtggcaagactgacaacacagactcccttacaaggtccccctccgtttttgcaaaatcaaaagagaacaagactaaggctaccaggtttgacccatctatggagaccctagaagatatgaagtacaaaccggacctaatccgagaggatgaatttagagatgagatccgcaacccgttgtaccaagagagggacacagaacccagggcctcaaacgcatcacgcctcctcccctccaaagagaagcccacaatgcactctctcaggctcgtcatagagagcagtcccctaagcagagctgagaaagcagcatatgtgaaatcattatccaagtgcaagacagaccaagaggttaaggcagtcatggaactcgtagaagaggacatagagtcactgaccaactag

SEQ ID NO: 4 - protein, 492 aa, Sendai virus
MSTQDNRSGEESRVSGRTSKPEAEAHAGNLDKQNIHRAFGGRTGTNSVSQDLGDGGDSGILENPPNERGYPRSGIEDENREMAAHPDKRGEDQAEGLPEEVRGGTSLPDEGEGGASNNGRSMEPGSSHSARVTGVLVIPSPELEEAVLRRNKRRPTNSGSKPLTPATVPGTRSPPLNRYNSTGSPPGKPPSTQDEHINSGDTPAVRVKDRKPPIGTRSVSDCPANGRPIHPGLETDSTKKGIGENTSSMKEMATLLTSLGVIQSAQEFESSRDASYVFARRALKSANYAEMTFNVCGLILSAEKSSARKVDENKQLLKQIQESVESFRDIYKRFSEYQKEQNSLLMSNLSTLHIITDRGGKTDNTDSLTRSPSVFAKSKENKTKATRFDPSMETLEDMKYKPDLIREDEFRDEIRNPLYQERDTEPRASNASRLLPSKEKPTMHSLRLVIESSPLSRAEKAAYVKSLSKCKTDQEVKAVMELVEEDIESLTN

SEQ ID NO: 5 - DNA, 1698 bp, Sendai virus
  source 1..1698: mol_type = other DNA
atgacagcatatatccagaggtcacagtgcatctcaacatcactactggttgttctcaccacattggtctcgtgtcagattcccagggataggctctctaacataggggtcatagtcgatgaagggaaatcactgaagatagctggatcccacgaatcgaggtacatagtactgagtctagttccgggggtagaccttgagaatgggtgcggaacagcccaggttatccagtacaagagcctactgaacaggctgttaatcccattgagggatgccttagatcttcaggaggctctgataactgtcaccaatgatacgacacaaaatgccggtcgtcgtcagaagagattcttcggtgctgtgattggtactatcgcacttggagtggcgacatcagcacagatcaccgcagggattgcactagccgaagcgagggaggccaaaagagacatagcgctcatcaaagaatcgatgacaaaaacacacaagtctatagaactgctgcaaaacgctgtgggggaacaaattcttgctctaaagacactccaggatttcgtgaatgatgagatcaaacccgcaataagcgaattaggctgtgagactgctgccttaagactgggtataaaattgacacagcattactccgggctgttaactgcgttcggctcgaatttcggaaccatcggagagaagagcctcacgctgcaggcgctgtcttcactttactctgctaacattactgagattatgaccacaatcaggacagggcagtctaacatctatgatgtcatttatacagaacagatcaaaggaacggtgatagatgtggatctagagagatacatggttaccctgtctgtgaagatccctattctttctgaagtcccaggtgtgctcatacacaaggcatcgtctatttcttacaacatagacggggaggaatggtatgtgactgtccccagccatatactcagtcgtgcttctttcttagggggtgcagacataaccgattgtgttgagtccagattgacctatatatgccccagggatcccgcacaactgatacctgacagccagcaaaagtgtatcctgggggacacaacaaggtgtcctgtcacaaaagttgtggacagccttatccccaagtttgcttttgtgaatgggggcgttgttgctaactgcatagcatccacatgtacctgcgggacaggccgaagaccaatcagtcaggatcgctctaaaggtgtagtattcctaacccatgacaactgtggtcttataggtgtcaatggggtagaattgtatgctaaccggagagggcacgatgccacttggggggtccagaacttgacagtcggtcctgcaattgctatcagacccgttgatatttctctcaaccttgctgatgctacgaatttcttgcaagactctaaggctgagcttgagaaagcacggaaaatcctctctgaggtaggtagatggtacaactcaagagagactgtgattacgatcatagtagttatggtcgtaatattggtggtcattatagtgatcgtcatcgtgctttatagactcaaaaggtcaatgctaatgggtaatccagatgaccgtataccgagggacacatatacattagagccgaagatcagacatatgtacacaaacggtgggtttgatgcgatggctgagaaaagatga

SEQ ID NO: 6 - protein, 565 aa, Sendai virus
MTAYIQRSQCISTSLLVVLTTLVSCQIPRDRLSNIGVIVDEGKSLKIAGSHESRYIVLSLVPGVDLENGCGTAQVIQYKSLLNRLLIPLRDALDLQEALITVTNDTTQNAGRRQKRFFGAVIGTIALGVATSAQITAGIALAEAREAKRDIALIKESMTKTHKSIELLQNAVGEQILALKTLQDFVNDEIKPAISELGCETAALRLGIKLTQHYSGLLTAFGSNFGTIGEKSLTLQALSSLYSANITEIMTTIRTGQSNIYDVIYTEQIKGTVIDVDLERYMVTLSVKIPILSEVPGVLIHKASSISYNIDGEEWYVTVPSHILSRASFLGGADITDCVESRLTYICPRDPAQLIPDSQQKCILGDTTRCPVTKVVDSLIPKFAFVNGGVVANCIASTCTCGTGRRPISQDRSKGVVFLTHDNCGLIGVNGVELYANRRGHDATWGVQNLTVGPAIAIRPVDISLNLADATNFLQDSKAELEKARKILSEVGRWYNSRETVITIIVVMVVILVVIIVIVIVLYRLKRSMLMGNPDDRIPRDTYTLEPKIRHMYTNGGFDAMAEKR

SEQ ID NO: 7 - DNA, 72 bp, Sendai virus
  source 1..72: mol_type = other DNA
actgtgattacgatcatagtagttatggtcgtaatattggtggtcattatagtgatcgtcatcgtgctttat

SEQ ID NO: 8 - protein, 24 aa, Sendai virus
TVITIIVVMVVILVVIIVIVIVLY

SEQ ID NO: 9 - DNA, 129 bp, synthetic construct
  misc_feature 1..129: note = n/a
  source 1..129: mol_type = other DNA
agactcaaaaggtcaatgctaatgggtaatccagatgaccgtataccgagggacacatatacattagagccgaagatcagacatatgtacacaaacggtgggtttgatgcgatggctgagaaaagatga

SEQ ID NO: 10 - protein, 42 aa, synthetic construct
  REGION 1..42: note = n/a
RLKRSMLMGNPDDRIPRDTYTLEPKIRHMYTNGGFDAMAEKR

SEQ ID NO: 11 - DNA, 1728 bp, Sendai virus
  source 1..1728: mol_type = other DNA
atggatggtgataggggcaaacgtgactcgtactggtctacctctcctagtggtagcactacaaaattagcatcaggttgggagaggtcaagtaaagttgacacatggttgctgattctctcattcacccagtgggctttgtcaattgccacagtgatcatctgtatcataatttctgctagacaagggtatagtatgaaagagtactcaatgactgtagaggcattgaacatgagcagcagggaggtgaaagagtcacttaccagtctaataaggcaagaggttatcgcaagggctgtcaacattcagagctctgtgcaaaccggaatcccagtcttgttgaacaaaaacagcagggatgtcatccagatgattgataagtcgtgcagcagacaagagctcactcagctctgtgagagtacgatcgcagtccaccatgccgagggaattgccccacttgagccacatagtttctggagatgccctgtcggagaaccgtatcttagctcagatcctaaaatctcattgctgcctggtccgagcttgttatctggttctacaacgatctctggatgtgttaggctcccttcactctcaattggcgaggcaatctatgcctattcatcaaatctcattacacaaggttgtgctgacatagggaaatcatatcaggtcctgcagctagggtacatatcactcaattcagatatgttccctgatcttaaccccgtagtgtcccacacttatgacatcaacgacaatcggaaatcatgctctgtggtggcaaccgggactaggggttatcagctttgctccatgccgactgtagacgaaagaaccgactactctagtgatggtatcgaggatctggtccttgatgtcctggatctcaaagggagcactaagtctcaccggtatcgcaacagcgaggtagatcttgatcacccgttctctgcactataccccagtgtaggcaacggcattgcaacagaaggctcattgatatttcttgggtatggtgggctaaccacccctctacagggtgatacaaaatgtaggacccaaggatgccaacaggtgtcgcaagacacatgcaatgaggctctgaaaattacatggctaggagggaaacaggtggtcagcgtgatcatccaggtcaatgactatctctcagagaggccaaagataagagtcacaaccattccaatcactcaaaactatctcggggcggaaggtagattattaaaattgggtgatcgggtgtacatctatacaagatcatcaggctggcactctcaactgcagataggagtacttgatgtcagccaccctttgactatcaactggacacctcatgaagccttgtctagaccaggaaatgaagagtgcaattggtacaatacgtgtccgaaggaatgcatatcaggcgtatacactgatgcttatccattgtcccctgatgcagctaacgtcgctaccgtcacgctatatgccaatacatcgcgtgtcaacccaacaatcatgtattctaacactactaacattataaatatgttaaggataaaggatgttcaattagaggctgcatataccacgacatcgtgtatcacgcattttggtaaaggctactgctttcacatcatcgagatcaatcagaagagcctgaataccttacagccgatgctctttaagactagcatccctaaattatgcaaggccgagtcttaa

SEQ ID NO: 12 - protein, 575 aa, synthetic construct
  REGION 1..575: note = n/a
MDGDRGKRDSYWSTSPSGSTTKLASGWERSSKVDTWLLILSFTQWALSIATVIICIIISARQGYSMKEYSMTVEALNMSSREVKESLTSLIRQEVIARAVNIQSSVQTGIPVLLNKNSRDVIQMIDKSCSRQELTQLCESTIAVHHAEGIAPLEPHSFWRCPVGEPYLSSDPKISLLPGPSLLSGSTTISGCVRLPSLSIGEAIYAYSSNLITQGCADIGKSYQVLQLGYISLNSDMFPDLNPVVSHTYDINDNRKSCSVVATGTRGYQLCSMPTVDERTDYSSDGIEDLVLDVLDLKGSTKSHRYRNSEVDLDHPFSALYPSVGNGIATEGSLIFLGYGGLTTPLQGDTKCRTQGCQQVSQDTCNEALKITWLGGKQVVSVIIQVNDYLSERPKIRVTTIPITQNYLGAEGRLLKLGDRVYIYTRSSGWHSQLQIGVLDVSHPLTINWTPHEALSRPGNEECNWYNTCPKECISGVYTDAYPLSPDAANVATVTLYANTSRVNPTIMYSNTTNIINMLRIKDVQLEAAYTTTSCITHFGKGYCFHIIEINQKSLNTLQPMLFKTSIPKLCKAES

SEQ ID NO: 13 - DNA, 111 bp, synthetic construct
  misc_feature 1..111: note = n/a
  source 1..111: mol_type = other DNA
atggatggtgataggggcaaacgtgactcgtactggtctacctctcctagtggtagcactacaaaattagcatcaggttgggagaggtcaagtaaagttgacacatggttg

SEQ ID NO: 14 - protein, 37 aa, synthetic construct
  REGION 1..37: note = n/a
MDGDRGKRDSYWSTSPSGSTTKLASGWERSSKVDTWL

SEQ ID NO: 15 - DNA, 63 bp, synthetic construct
  misc_feature 1..63: note = n/a
  source 1..63: mol_type = other DNA
ctgattctctcattcacccagtgggctttgtcaattgccacagtgatcatctgtatcataatt

SEQ ID NO: 16 - protein, 21 aa, synthetic construct
  REGION 1..21: note = n/a
LILSFTQWALSIATVIICIII

SEQ ID NO: 17 - DNA, 1554 bp, synthetic construct
  misc_feature 1..1554: note = n/a
  source 1..1554: mol_type = other DNA
tctgctagacaagggtatagtatgaaagagtactcaatgactgtagaggcattgaacatgagcagcagggaggtgaaagagtcacttaccagtctaataaggcaagaggttatcgcaagggctgtcaacattcagagctctgtgcaaaccggaatcccagtcttgttgaacaaaaacagcagggatgtcatccagatgattgataagtcgtgcagcagacaagagctcactcagctctgtgagagtacgatcgcagtccaccatgccgagggaattgccccacttgagccacatagtttctggagatgccctgtcggagaaccgtatcttagctcagatcctaaaatctcattgctgcctggtccgagcttgttatctggttctacaacgatctctggatgtgttaggctcccttcactctcaattggcgaggcaatctatgcctattcatcaaatctcattacacaaggttgtgctgacatagggaaatcatatcaggtcctgcagctagggtacatatcactcaattcagatatgttccctgatcttaaccccgtagtgtcccacacttatgacatcaacgacaatcggaaatcatgctctgtggtggcaaccgggactaggggttatcagctttgctccatgccgactgtagacgaaagaaccgactactctagtgatggtatcgaggatctggtccttgatgtcctggatctcaaagggagcactaagtctcaccggtatcgcaacagcgaggtagatcttgatcacccgttctctgcactataccccagtgtaggcaacggcattgcaacagaaggctcattgatatttcttgggtatggtgggctaaccacccctctacagggtgatacaaaatgtaggacccaaggatgccaacaggtgtcgcaagacacatgcaatgaggctctgaaaattacatggctaggagggaaacaggtggtcagcgtgatcatccaggtcaatgactatctctcagagaggccaaagataagagtcacaaccattccaatcactcaaaactatctcggggcggaaggtagattattaaaattgggtgatcgggtgtacatctatacaagatcatcaggctggcactctcaactgcagataggagtacttgatgtcagccaccctttgactatcaactggacacctcatgaagccttgtctagaccaggaaatgaagagtgcaattggtacaatacgtgtccgaaggaatgcatatcaggcgtatacactgatgcttatccattgtcccctgatgcagctaacgtcgctaccgtcacgctatatgccaatacatcgcgtgtcaacccaacaatcatgtattctaacactactaacattataaatatgttaaggataaaggatgttcaattagaggctgcatataccacgacatcgtgtatcacgcattttggtaaaggctactgctttcacatcatcgagatcaatcagaagagcctgaataccttacagccgatgctctttaagactagcatccctaaattatgcaaggccgagtcttaa

SEQ ID NO: 18 - protein, 517 aa, synthetic construct
  REGION 1..517: note = n/a
SARQGYSMKEYSMTVEALNMSSREVKESLTSLIRQEVIARAVNIQSSVQTGIPVLLNKNSRDVIQMIDKSCSRQELTQLCESTIAVHHAEGIAPLEPHSFWRCPVGEPYLSSDPKISLLPGPSLLSGSTTISGCVRLPSLSIGEAIYAYSSNLITQGCADIGKSYQVLQLGYISLNSDMFPDLNPVVSHTYDINDNRKSCSVVATGTRGYQLCSMPTVDERTDYSSDGIEDLVLDVLDLKGSTKSHRYRNSEVDLDHPFSALYPSVGNGIATEGSLIFLGYGGLTTPLQGDTKCRTQGCQQVSQDTCNEALKITWLGGKQVVSVIIQVNDYLSERPKIRVTTIPITQNYLGAEGRLLKLGDRVYIYTRSSGWHSQLQIGVLDVSHPLTINWTPHEALSRPGNEECNWYNTCPKECISGVYTDAYPLSPDAANVATVTLYANTSRVNPTIMYSNTTNIINMLRIKDVQLEAAYTTTSCITHFGKGYCFHIIEINQKSLNTLQPMLFKTSIPKLCKAES

SEQ ID NO: 19 - DNA, 3822 bp, SARS-CoV-2
  source 1..3822: mol_type = other DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 20 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 21 - DNA, 3639 bp, synthetic construct
  misc_feature 1..3639: note = n/a
  source 1..3639: mol_type = other DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggcca

SEQ ID NO: 22 - protein, 1213 aa, synthetic construct
  REGION 1..1213: note = n/a
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 23 - DNA, 63 bp, synthetic construct
  misc_feature 1..63: note = n/a
  source 1..63: mol_type = other DNA
tggtacatttggctaggttttatagctggcttgattgccatagtaatggtgacaattatgctt

SEQ ID NO: 24 - protein, 21 aa, synthetic construct
  REGION 1..21: note = n/a
WYIWLGFIAGLIAIVMVTIML

SEQ ID NO: 25 - DNA, 120 bp, synthetic construct
  misc_feature 1..120: note = n/a
  source 1..120: mol_type = other DNA
tgctgtatgaccagttgctgtagttgtctcaagggctgttgttcttgtggatcctgctgcaaatttgatgaagacgactctgagccagtgctcaaaggagtcaaattacattacacataa

SEQ ID NO: 26 - protein, 39 aa, synthetic construct
  REGION 1..39: note = n/a
CCMTSCCSCLKGCCSCGSCCKFDEDDSEPVLKGVKLHYT

SEQ ID NO: 27 - DNA, 3840 bp, synthetic construct
  misc_feature 1..3840: note = n/a
  source 1..3840: mol_type = other DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggagttctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccaactgtgattacgatcatagtagttatggtcgtaatattggtggtcattatagtgatcgtcatcgtgctttatagactcaaaaggtcaatgctaatgggtaatccagatgaccgtataccgagggacacatatacattagagccgaagatcagacatatgtacacaaacggtgggtttgatgcgatggctgagaaaagatga

SEQ ID NO: 28 - DNA, 3639 bp, synthetic construct
  misc_feature 1..3639: note = n/a
  source 1..3639: mol_type = other DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggagttctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggcca

SEQ ID NO: 29 - DNA, 201 bp, synthetic construct
  misc_feature 1..201: note = n/a
  source 1..201: mol_type = other DNA
actgtgattacgatcatagtagttatggtcgtaatattggtggtcattatagtgatcgtcatcgtgctttatagactcaaaaggtcaatgctaatgggtaatccagatgaccgtataccgagggacacatatacattagagccgaagatcagacatatgtacacaaacggtgggtttgatgcgatggctgagaaaagatga

SEQ ID NO: 30 - DNA, 3840 bp, synthetic construct
  misc_feature 1..3840: note = n/a
  source 1..3840: mol_type = other DNA
atgttcgtcttcctggtgctgctgcccctggtctcttctcagtgcgtgaacctgacaactcggactcagctgccccctgcctataccaattccttcacacggggcgtgtactatccagacaaggtgtttagaagctccgtgctgcactccacacaggatctgtttctgcccttcttttctaacgtgacctggttccacgccatccacgtgagcggcaccaatggcacaaagcggttcgacaatccagtgctgccctttaacgatggcgtgtacttcgccagcaccgagaagtccaacatcatcagaggctggatctttggcaccacactggactctaagacacagagcctgctgatcgtgaacaatgccaccaacgtggtcatcaaggtgtgcgagttccagttttgtaatgatcctttcctgggcgtgtactatcacaagaacaataagtcctggatggagtctgagtttagggtgtattctagcgccaacaattgcacatttgagtacgtgtcccagccattcctgatggacctggagggcaagcagggcaatttcaagaacctgagggagttcgtgtttaagaatatcgatggctacttcaaaatctactccaagcacacccctatcaacctggtgcgcgacctgccacagggcttctctgccctggagccactggtggatctgcccatcggcatcaacatcacccggtttcagacactgctggccctgcacagaagctacctgacacctggcgactcctctagcggatggaccgccggcgccgccgcctactatgtgggctatctgcagccaaggaccttcctgctgaagtacaacgagaatggcaccatcacagacgcagtggattgcgccctggaccccctgtctgagacaaagtgtacactgaagagctttaccgtggagaagggcatctatcagacaagcaatttcagggtgcagcccaccgagtccatcgtgcgctttccaaatatcacaaacctgtgcccctttggcgaggtgttcaacgcaaccaggttcgccagcgtgtacgcatggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcaccaggacagacaggcaagatcgccgactacaattataagctgcccgacgatttcaccggctgcgtgatcgcctggtcctctaacaatctggattctaaagtgggcggcaactacaattatctgtaccggctgtttagaaagagcaacctgaagcctttcgagagggacatctccacagaaatctaccaggccggctctaccccatgcaatggcgtggagggctttaactgttatttccccctgcagagctacggcttccagcctaccaatggcgtgggctatcagccataccgcgtggtggtgctgtcctttgagctgctgcacgcaccagcaacagtgtgcggacctaagaagtctaccaatctggtgaagaacaagtgcgtgaacttcaacttcaacggcctgaccggcacaggcgtgctgaccgagtccaacaagaagttcctgccctttcagcagttcggcagggacatcgcagataccacagacgccgtgcgcgacccccagaccctggagatcctggacatcacaccttgctccttcggcggcgtgtctgtgatcacacctggcaccaatacaagcaaccaggtggccgtgctgtatcagggcgtgaattgtaccgaggtgccagtggcaatccacgcagaccagctgaccccaacatggcgggtgtactctaccggcagcaacgtgttccagacaagagccggatgcctgatcggagcagagcacgtgaacaatagctatgagtgcgacatccccatcggcgccggcatctgtgcctcctaccagacccagacaaactcccctaggagagccaggtctgtggccagccagtccatcatcgcctataccatgagcctgggcgccgagaacagcgtggcctactccaacaattctatcgccatccccaccaacttcacaatctccgtgaccacagagatcctgcctgtgagcatgaccaagacatccgtggactgcacaatgtatatctgtggcgatagcaccgagtgctccaacctgctgctgcagtacggctctttttgtacccagctgaatcgcgccctgacaggaatcgcagtggagcaggacaagaacacacaggaggtgttcgcccaggtgaagcaaatctacaagaccccacccatcaaggactttggcggcttcaacttcagccagatcctgcccgatcctagcaagccttccaagcggtcttttatcgaggacctgctgttcaacaaggtgaccctggccgatgccggcttcatcaagcagtatggcgattgcctgggcgacatcgcagcccgggacctgatctgcgcccagaagtttaatggcctgaccgtgctgcctccactgctgacagatgagatgatcgcccagtacacatctgccctgctggccggcaccatcacaagcggatggaccttcggcgccggcgccgccctgcagatcccctttgccatgcagatggcctatagattcaacggcatcggcgtgacccagaatgtgctgtacgagaaccagaagctgatcgccaatcagtttaactccgccatcggcaagatccaggactctctgagcagcaccgccagcgccctgggcaagctgcaggatgtggtgaatcagaacgcccaggccctgaataccctggtgaagcagctgtctagcaacttcggcgccatctcctctgtgctgaatgacatcctgagccggctggacaaggtggaggcagaggtgcagatcgaccggctgatcacaggcagactgcagagcctgcagacctacgtgacacagcagctgatcagggcagcagagatcagggcctccgccaatctggcagcaaccaagatgagcgagtgcgtgctgggccagtccaagagagtggacttttgtggcaagggctatcacctgatgtctttcccacagagcgcccctcacggcgtggtgtttctgcacgtgacctacgtgcctgcccaggagaagaacttcaccacagccccagccatctgccacgatggcaaggcccactttcccagggagggcgtgttcgtgtctaacggcacccactggtttgtgacacagcgcaatttctacgagcctcagatcatcaccacagacaatacatttgtgagcggcaactgtgacgtggtcatcggcatcgtgaacaataccgtgtatgatcctctgcagccagagctggactcttttaaggaggagctggataagtacttcaagaatcacaccagccccgacgtggatctgggcgacatctctggcatcaatgccagcgtggtgaacatccagaaggagatcgaccggctgaacgaggtggccaagaatctgaacgagtccctgatcgatctgcaggagctgggcaagtatgagcagtacatcaagtggccaaccgtgatcacaatcatcgtggtcatggtggtcatcctggtggtcatcatcgtgatcgtgatcgtgctgtataggctgaagcgcagcatgctgatgggcaaccctgacgatagaatcccccgggacacctacacactggagcccaagatcagacacatgtacactaatggaggatttgacgcaatggcagaaaagagatga

SEQ ID NO: 31 - protein, 1279 aa, synthetic construct
  REGION 1..1279: note = n/a
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWSSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPTVITIIVVMVVILVVIIVIVIVLYRLKRSMLMGNPDDRIPRDTYTLEPKIRHMYTNGGFDAMAEKR

SEQ ID NO: 32 - protein, 1213 aa, synthetic construct
  REGION 1..1213: note = n/a
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWSSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 33 - protein, 66 aa, synthetic construct
  REGION 1..66: note = n/a
TVITIIVVMVVILVVIIVIVIVLYRLKRSMLMGNPDDRIPRDTYTLEPKIRHMYTNGGFDAMAEKR

SEQ ID NO: 34 - DNA, 3840 bp, synthetic construct
  misc_feature 1..3840: note = n/a
  source 1..3840: mol_type = other DNA
atgtttgtctttctggtcctgctgcccctggtctcatcacagtgcgtcaacctgactacacgaacacagctgccccctgcttacaccaattccttcacccggggcgtgtactatccagacaaggtgtttagaagctccgtgctgcactccacacaggatctgtttctgcccttcttttctaacgtgacctggttccacgccatccacgtgagcggcaccaatggcacaaagcggttcgacaatccagtgctgccctttaacgatggcgtgtacttcgccagcaccgagaagtccaacatcatcagaggctggatctttggcaccacactggactctaagacacagagcctgctgatcgtgaacaatgccaccaacgtggtcatcaaggtgtgcgagttccagttttgtaatgatcctttcctgggcgtgtactatcacaagaacaataagtcctggatggagtctgagtttagggtgtactctagcgccaacaattgcacatttgagtatgtgtcccagccattcctgatggacctggagggcaagcagggcaatttcaagaacctgagggagttcgtgtttaagaacatcgatggctacttcaagatctactccaagcacacccctatcaacctggtgcgcgacctgccacagggcttctctgccctggagccactggtggatctgcccatcggcatcaacatcacccggtttcagacactgctggccctgcacagaagctacctgacacctggcgactcctctagcggatggacagcaggagcagcagcctactatgtgggctacctgcagccaaggaccttcctgctgaagtataacgagaatggcaccatcacagacgccgtggattgcgccctggatccactgtctgagacaaagtgtacactgaagagctttaccgtggagaagggcatctaccagacaagcaatttcagggtgcagcccaccgagtccatcgtgcgctttccaaatatcacaaacctgtgcccctttggcgaggtgttcaacgccacccgcttcgccagcgtgtatgcctggaataggaagcgcatctccaactgtgtggccgactattctgtgctgtacaacagcgcctccttctctacctttaagtgttacggcgtgagccccacaaagctgaatgatctgtgttttaccaacgtgtatgccgattccttcgtgattaggggcgacgaggtgcgccagattgcaccaggacagacaggcaagatcgccgactacaattataagctgcccgacgatttcaccggctgcgtgatcgcctggtcctctaacaatctggattctaaagtgggcggcaactacaattatctgtaccggctgtttagaaagagcaacctgaagcctttcgagagggacatctccacagagatctaccaggccggctctaccccatgcaatggcgtgaagggctttaactgttatttccccctgcagagctacggcttccagcctacctatggcgtgggctatcagccataccgcgtggtggtgctgtcctttgagctgctgcacgcacctgcaacagtgtgtggacctaagaagtctaccaatctggtgaagaacaagtgtgtgaacttcaattttaatggcctgacaggaacaggagtgctgaccgagtccaacaagaagttcctgccctttcagcagttcggcagggacattgcagatacaaccgatgccgtgcgcgacccccagaccctggagatcctggatatcacaccttgctccttcggcggcgtgtctgtgatcacacctggcaccaatacaagcaaccaggtggccgtgctgtaccagggcgtgaattgtacagaggtgccagtggcaattcacgcagaccagctgaccccaacatggcgggtgtattctaccggcagcaacgtgttccagacaagagccggatgcctgattggagcagagcacgtgaacaatagctacgagtgcgatatccccatcggcgccggcatctgtgcctcctatcagacccagacaaactcccctcggagagcccggtctgtggcaagccagtccatcatcgcctacaccatgagcctgggcgccgagaacagcgtggcctattccaacaattctatcgccatccccaccaacttcacaatctccgtgaccacagagatcctgcctgtgagcatgaccaagacatccgtggactgcacaatgtacatctgtggcgatagcaccgagtgctccaacctgctgctgcagtatggctctttttgtacccagctgaatagagccctgacaggcatcgccgtggagcaggacaagaacacacaggaggtgttcgcccaggtgaagcagatctacaagacccctcccatcaaggactttggcggcttcaattttagccagatcctgcccgatcctagcaagccttccaagcggtcttttatcgaggacctgctgttcaacaaggtgaccctggccgatgccggcttcatcaagcagtatggcgattgcctgggcgacattgcagcccgggacctgatctgtgcccagaagtttaatggcctgaccgtgctgccacccctgctgacagatgagatgatcgcccagtatacatctgccctgctggcaggaaccatcacatctggatggacctttggagcaggagcagcactgcagatcccctttgccatgcagatggcctacaggttcaacggcatcggcgtgacccagaatgtgctgtatgagaaccagaagctgatcgccaatcagtttaactccgccatcggcaagatccaggactctctgagctccacagcaagcgccctgggcaagctgcaggatgtggtgaatcagaacgcccaggccctgaataccctggtgaagcagctgtctagcaacttcggcgccatctcctctgtgctgaatgatatcctgagccggctggaccctccagaggcagaggtgcagatcgaccggctgatcacaggcagactgcagagcctgcagacctacgtgacacagcagctgattagggcagcagaaattagagcatctgcaaatctggcagcaaccaagatgagcgagtgcgtgctgggccagtccaagagagtggacttttgtggcaagggctaccacctgatgtcttttcctcagagcgcccctcacggagtggtgtttctgcacgtgacctatgtgcctgcccaggagaagaacttcaccacagccccagccatctgccacgatggcaaggcccactttcccagggagggcgtgttcgtgtctaacggcacccactggtttgtgacacagcgcaatttctacgagcctcagatcatcaccacagacaatacatttgtgagcggcaactgtgatgtggtcatcggcatcgtgaacaataccgtgtacgatcctctgcagccagagctggactcttttaaggaggagctggataagtatttcaagaatcacaccagcccagacgtggatctgggcgacatctctggcatcaatgccagcgtggtgaacatccagaaggagatcgaccgcctgaacgaggtggccaagaatctgaacgagtccctgatcgatctgcaggagctgggcaagtatgagcagtacatcaagtggcccaccgtgatcacaatcatcgtggtcatggtggtcatcctggtggtcatcatcgtgatcgtgatcgtgctgtacaggctgaagcgcagcatgctgatgggcaaccctgacgatcggatcccaagagatacctatacactggagcccaagattcggcacatgtacactaatggcggctttgacgcaatggcagaaaaacgatga

SEQ ID NO: 35 - protein, 1279 aa, synthetic construct
  REGION 1..1279: note = n/a
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWSSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPTVITIIVVMVVILVVIIVIVIVLYRLKRSMLMGNPDDRIPRDTYTLEPKIRHMYTNGGFDAMAEKR

SEQ ID NO: 36 - DNA, 3891 bp, synthetic construct
  misc_feature 1..3891: note = n/a
  source 1..3891: mol_type = other DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggagttctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcagactcaaaaggtcaatgctaatgggtaatccagatgaccgtataccgagggacacatatacattagagccgaagatcagacatatgtacacaaacggtgggtttgatgcgatggctgagaaaagatga

SEQ ID NO: 37 - protein, 1296 aa, synthetic construct
  REGION 1..1296: note = n/a
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWSSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCRLKRSMLMGNPDDRIPRDTYTLEPKIRHMYTNGGFDAMAEKR

SEQ ID NO: 38 - DNA, 3639 bp, synthetic construct; identical to SEQ ID NO: 28
  misc_feature 1..3639: note = n/a
  source 1..3639: mol_type = other DNA

SEQ ID NO: 39 - protein, 1213 aa, synthetic construct; identical to SEQ ID NO: 32
  REGION 1..1213: note = n/a

SEQ ID NO: 40 - DNA, 123 bp, synthetic construct
  misc_feature 1..123: note = n/a
  source 1..123: mol_type = other DNA
tggtacatttggctaggttttatagctggcttgattgccatagtaatggtgacaattatgctttgctgtatgaccagttgctgtagttgtctcaagggctgttgttcttgtggatcctgctgc

SEQ ID NO: 41 - protein, 41 aa, synthetic construct
  REGION 1..41: note = n/a
WYIWLGFIAGLIAIVMVTIMLCCMTSCCSCLKGCCSCGSCC

SEQ ID NO: 42 - DNA, 129 bp, synthetic construct; identical to SEQ ID NO: 9
  misc_feature 1..129: note = n/a
  source 1..129: mol_type = other DNA

SEQ ID NO: 43 - protein, 42 aa, synthetic construct; identical to SEQ ID NO: 10
  REGION 1..42: note = n/a

SEQ ID NO: 44 - DNA, 3891 bp, synthetic construct
  misc_feature 1..3891: note = n/a
  source 1..3891: mol_type = other DNA
atgttcgtcttcctggtgctgctgcccctggtctcttctcagtgcgtgaacctgacaactcggactcagctgccccctgcctataccaattccttcacacggggcgtgtactatccagacaaggtgtttagaagctccgtgctgcactccacacaggatctgtttctgcccttcttttctaacgtgacctggttccacgccatccacgtgagcggcaccaatggcacaaagcggttcgacaatccagtgctgccctttaacgatggcgtgtacttcgccagcaccgagaagtccaacatcatcagaggctggatctttggcaccacactggactctaagacacagagcctgctgatcgtgaacaatgccaccaacgtggtcatcaaggtgtgcgagttccagttttgtaatgatcctttcctgggcgtgtactatcacaagaacaataagtcctggatggagtctgagtttagggtgtattctagcgccaacaattgcacatttgagtacgtgtcccagccattcctgatggacctggagggcaagcagggcaatttcaagaacctgagggagttcgtgtttaagaatatcgatggctacttcaaaatctactccaagcacacccctatcaacctggtgcgcgacctgccacagggcttctctgccctggagccactggtggatctgcccatcggcatcaacatcacccggtttcagacactgctggccctgcacagaagctacctgacacctggcgactcctctagcggatggaccgccggcgccgccgcctactatgtgggctatctgcagccaaggaccttcctgctgaagtacaacgagaatggcaccatcacagacgcagtggattgcgccctggaccccctgtctgagacaaagtgtacactgaagagctttaccgtggagaagggcatctatcagacaagcaatttcagggtgcagcccaccgagtccatcgtgcgctttccaaatatcacaaacctgtgcccctttggcgaggtgttcaacgcaaccaggttcgccagcgtgtacgcatggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcaccaggacagacaggcaagatcgccgactacaattataagctgcccgacgatttcaccggctgcgtgatcgcctggtcctctaacaatctggattctaaagtgggcggcaactacaattatctgtaccggctgtttagaaagagcaacctgaagcctttcgagagggacatctccacagaaatctaccaggccggctctaccccatgcaatggcgtggagggctttaactgttatttccccctgcagagctacggcttccagcctaccaatggcgtgggctatcagccataccgcgtggtggtgctgtcctttgagctgctgcacgcaccagcaacagtgtgcggacctaagaagtctaccaatctggtgaagaacaagtgcgtgaacttcaacttcaacggcctgaccggcacaggcgtgctgaccgagtccaacaagaagttcctgccctttcagcagttcggcagggacatcgcagataccacagacgccgtgcgcgacccccagaccctggagatcctggacatcacaccttgctccttcggcggcgtgtctgtgatcacacctggcaccaatacaagcaaccaggtggccgtgctgtatcagggcgtgaattgtaccgaggtgccagtggcaatccacgcagaccagctgaccccaacatggcgggtgtactctaccggcagcaacgtgttccagacaagagccggatgcctgatcggagcagagcacgtgaacaatagctatgagtgcgacatccccatcggcgccggcatctgtgcctcctaccagacccagacaaactcccctaggagagccaggtctgtggccagccagtccatcatcgcctataccatgagcctgggcgccgagaacagcgtggcctactccaacaattctatcgccatccccaccaacttcacaatctccgtgaccacagagatcctgcctgtgagcatgaccaagacatccgtggactgcacaatgtatatctgtggcgatagcaccgagtgctccaacctgctgctgcagtacggctctttttgtacccagctgaatcgcgccctgacaggaatcgcagtggagcaggacaagaacacacaggaggtgttcgcccaggtgaagcaaatctacaagaccccacccatcaaggactttggcggcttcaacttcagccagatcctgcccgatcctagcaagccttccaagcggtcttttatcgaggacctgctgttcaacaaggtgaccctggccgatgccggcttcatcaagcagtatggcgattgcctgggcgacatcgcagcccgggacctgatctgcgcccagaagtttaatggcctgaccgtgctgcctccactgctgacagatgagatgatcgcccagtacacatctgccctgctggccggcaccatcacaagcggatggaccttcggcgccggcgccgccctgcagatcccctttgccatgcagatggcctatagattcaacggcatcggcgtgacccagaatgtgctgtacgagaaccagaagctgatcgccaatcagtttaactccgccatcggcaagatccaggactctctgagcagcaccgccagcgccctgggcaagctgcaggatgtggtgaatcagaacgcccaggccctgaataccctggtgaagcagctgtctagcaacttcggcgccatctcctctgtgctgaatgacatcctgagccggctggacaaggtggaggcagaggtgcagatcgaccggctgatcacaggcagactgcagagcctgcagacctacgtgacacagcagctgatcagggcagcagagatcagggcctccgccaatctggcagcaaccaagatgagcgagtgcgtgctgggccagtccaagagagtggacttttgtggcaagggctatcacctgatgtctttcccacagagcgcccctcacggcgtggtgtttctgcacgtgacctacgtgcctgcccaggagaagaacttcaccacagccccagccatctgccacgatggcaaggcccactttcccagggagggcgtgttcgtgtctaacggcacccactggtttgtgacacagcgcaatttctacgagcctcagatcatcaccacagacaatacatttgtgagcggcaactgtgacgtggtcatcggcatcgtgaacaataccgtgtatgatcctctgcagccagagctggactcttttaaggaggagctggataagtacttcaagaatcacaccagccccgacgtggatctgggcgacatctctggcatcaatgccagcgtggtgaacatccagaaggagatcgaccggctgaacgaggtggccaagaatctgaacgagtccctgatcgatctgcaggagctgggcaagtatgagcagtacatcaagtggccatggtacatttggctaggttttatagctggcttgattgccatagtaatggtgacaattatgctttgctgtatgaccagttgctgtagttgtctcaagggctgttgttcttgtggatcctgctgcagactcaaaaggtcaatgctaatgggtaatccagatgaccgtataccgagggacacatatacattagagccgaagatcagacatatgtacacaaacggtgggtttgatgcgatggctgagaaaagatga

SEQ ID NO: 45 - protein, 1296 aa, synthetic construct; identical to SEQ ID NO: 37
  REGION 1..1296: note = n/a

SEQ ID NO: 46 - DNA, 3885 bp, synthetic construct
  misc_feature 1..3885: note = n/a
  source 1..3885: mol_type = other DNA
atgtttgtctttttcgtcctgctgcccctggtctcaagccagtgcgtgaatctgactactagaactcagctgccacccgcctatactaatagcttcaccaggggcgtgtactatcccgacaaggtgtttcgcagctccgtgctgcactccacacaggatctgtttctgcctttcttttccaatgtgacctggttccacgtgatctctggcaccaacggcacaaagcggttcgacaatcccgtgctgccttttaacgatggcgtgtacttcgccagcatcgagaagtccaacatcatcagaggctggatctttggcaccacactggactctaagacacagagcctgctgatcgtgaacaatgccaccaacgtggtcatcaaggtgtgcgagttccagttttgtaacgatccattcctggaccacaagaacaataagtcctggatggagtctgagtttcgggtgtactctagcgccaacaattgcacctttgagtatgtgagccagcccttcctgatggacctggagggcaagcagggcaatttcaagaacctgagggagttcgtgtttaagaatatcgatggctacttcaagatctactctaagcacacccctatcatcgtgcgcgagcctgaggacctgccacagggcttcagcgccctggagccactggtggatctgcccatcggcatcaacatcaccaggtttcagacactgctggccctgcaccgctcctacctgacaccaggcgattcctctagcggatggaccgcaggagctgccgcctactatgtgggctacctgcagccccggaccttcctgctgaagtataacgagaatggcaccatcacagacgcagtggattgcgccctggaccccctgtctgagacaaagtgtacactgaagagctttaccgtggagaagggcatctaccagacaagcaatttccgggtgcagcctaccgagtccatcgtgagatttccaaatatcacaaacctgtgcccctttgacgaggtgttcaacgccaccagattcgccagcgtgtatgcctggaataggaagcgcatctccaactgcgtggccgattattctgtgctgtacaacctggcccctttctttacctttaagtgttacggcgtgtccccaacaaagctgaatgacctgtgctttaccaacgtgtatgccgattctttcgtgatcaggggcgacgaggtgcgccagatcgcaccaggacagacaggcaatatcgccgactacaactataagctgcccgacgatttcaccggctgcgtgatcgcctggaacagcaataagctggattctaaggtgagcggcaactacaattatctgtaccggctgtttagaaagtccaatctgaagcctttcgagcgggacatctctacagagatctaccaggccggcaataagccatgcaacggcgtggccggctttaactgttatttccctctgcggtcctactctttcagaccaacctatggcgtgggccaccagccctatagagtggtggtgctgagctttgagctgctgcacgcaccagcaacagtgtgcggacctaagaagtccaccaatctggtgaagaacaagtgcgtgaacttcaacttcaacggcctgaagggcaccggcgtgctgacagagagcaacaagaagttcctgccttttcagcagttcggccgggacatcgcagataccacagacgccgtgcgcgacccccagaccctggagatcctggatatcacaccatgctccttcggcggcgtgtctgtgatcacaccaggcaccaatacatccaaccaggtggccgtgctgtaccagggcgtgaattgtaccgaggtgcccgtggcaatccacgcagatcagctgacccctacatggagggtgtattccaccggctctaacgtgttccagacacgcgccggatgcctgatcggagccgagtatgtgaacaatagctacgagtgcgacatccccatcggcgccggcatctgtgcctcctaccagacccagacaaagtcccacaggagagcaaggtctgtggcaagccagtccatcatcgcctacaccatgagcctgggcgccgagaacagcgtggcctatagcaacaattccatcgccatccccaccaacttcacaatcagcgtgaccacagagatcctgcctgtgagcatgaccaagacatccgtggactgcacaatgtacatctgtggcgatagcaccgagtgctccaatctgctgctgcagtatggctccttttgtacccagctgaagcgcgccctgacaggaatcgcagtggagcaggataagaacacacaggaggtgttcgcccaggtgaagcagatctacaagaccccccctatcaagtattttggcggcttcaacttcagccagatcctgcctgacccatctaagccaagcaagaggtcctttatcgaggacctgctgttcaacaaggtgaccctggccgatgccggcttcatcaagcagtacggcgattgcctgggcgacatcgcagcccgcgacctgatctgtgcccagaagtttaagggcctgaccgtgctgccaccactgctgacagatgagatgatcgcccagtatacatctgccctgctggcaggcaccatcacaagcggatggaccttcggcgcaggagccgccctgcagatcccctttgccatgcagatggcctaccggttcaacggcatcggcgtgacccagaatgtgctgtatgagaaccagaagctgatcgccaatcagtttaacagcgccatcggcaagatccaggactctctgtcctctacagccagcgccctgggcaagctgcaggatgtggtgaatcacaacgcccaggccctgaataccctggtgaagcagctgagctccaagttcggcgccatctctagcgtgctgaacgatatcttttccaggctggacaaggtggaggccgaggtgcagatcgacaggctgatcacaggccgcctgcagtccctgcagacctacgtgacacagcagctgatccgggccgccgagatcagagcctctgccaatctggccgccaccaagatgagcgagtgcgtgctgggacagtccaagagggtggatttttgtggcaagggctaccacctgatgtctttcccacagagcgcccctcacggagtggtgtttctgcacgtgacctatgtgccagcccaggagaagaacttcaccacagcaccagcaatctgccacgacggcaaggcacactttcctcgggagggcgtgttcgtgtctaatggcacccactggtttgtgacacagagaaacttctacgagccacagatcatcaccacagacaatacattcgtgtccggcaactgtgacgtggtcatcggcatcgtgaacaataccgtgtacgatccactgcagcccgagctggactcttttaaggaggagctggataagtatttcaagaatcacaccagccccgacgtggatctgggcgatatctctggcatcaatgccagcgtggtgaacatccagaaggagatcgacagactgaacgaggtggccaagaatctgaacgagagcctgatcgatctgcaggagctgggcaagtatgagcagtacatcaagtggccttggtacatctggctgggcttcatcgccggcctgatcgccatcgtgatggtgaccatcatgctgtgctgtatgacatcttgctgtagctgcctgaagggctgctgttcctgtggctcttgctgccggctgaagcggagcatgctgatgggcaaccctgacgatagaatcccccgggacacctatacactggagcccaagattcggcacatgtatactaatggggggtttgatgctatggcagagaaaagataatga

SEQ ID NO: 47 - protein, 1293 aa, synthetic construct
  REGION 1..1293: note = n/a
MFVFFVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCRLKRSMLMGNPDDRIPRDTYTLEPKIRHMYTNGGFDAMAEKR

(19) United States
(12) Patent Application Publication
(10) Pub. No.: US 2026/0284178 A1
(43) Pub. Date: Sep. 24, 2026

(54) VACCINE WITH REDUCED ANTI-VECTOR ANTIGENICITY

(71) Applicant: MAX-PLANCK-GESELLSCHAFT ZUR FORDERUNG DER WISSENSCHAFTEN E.V., München (DE)

(72) Inventors: Reinhard FASSLER, Martinsried (DE); Wolfgang NEUBERT, Martinsried (DE); Marian WIEGAND, Munchen (DE); Ulrich M. LAUER, Tübingen (DE)

(21) Appl. No.: 18/868,571

(22) PCT Filed: May 25, 2023
(86) PCT No.: PCT/EP2023/064144

§ 371 Date: Nov. 22, 2024

(30) Foreign Application Priority Data
May 25, 2022 (EP) 22175583.8

(51) Int. Cl. A61K 39/215; A61K 39/00; C07K 14/005; C12N 15/86

(52) U.S. Cl. CPC A61K 39/215; C07K 14/005; C12N 15/86; A61K 2039/5256; C12N 2760/18843; C12N 2770/20022; C12N 2770/20034

(57) ABSTRACT

The present invention relates to a recombinant replication-deficient or replication competent negative-strand RNA virus, (i) capable of displaying a heterologous antigen on the surface, and thus capable of inducing mucosal immunity in host, (ii) after entering a host cell, producing a heterologous antigen, thereby improving immune response of the host to the antigen, and (iii) having a reduced anti-vector antigenicity in comparison to the wildtype vector, thus reducing an immune response of the host to the vector.

The inventors constructed a recombinant replication-competent or replication-deficient negative-strand RNA virus, (i) capable of displaying a heterologous antigen on the surface, (ii) capable of inducing mucosal immunity in host, (iii) after entering a host cell, producing a heterologous antigen, thereby improving immune response of the host to the antigen, and (iv) having a reduced anti-vector antigenicity in comparison to the wildtype vector, thus reducing an immune response of the host to the vector, i.e. inducing less anti-vector immunity.

The present invention relates to a recombinant replication-deficient negative-strand RNA virus, comprising in its genome: (a) a nucleotide sequence encoding a P polypeptide, said nucleotide sequence being modified in comparison to the wildtype form, said modification leading to replication deficiency of the negative-strand RNA virus, and (b) at least one nucleotide sequence encoding at least one heterologous polypeptide, wherein the at least one heterologous polypeptide is displayed on the surface of the virus particle, and can be expressed in infected cells.

Another aspect of the inventions relates to a recombinant replication-competent negative-strand RNA virus, comprising in its genome: (a) a nucleotide sequence encoding a P polypeptide, wherein the P protein exerts its natural function in the viral replication cycle, and (b) at least one nucleotide sequence encoding at least one heterologous polypeptide, wherein the at least one heterologous polypeptide is displayed on the surface of the virus particle, and can be expressed and replicated in infected cells.

Other aspects of the invention relate to a pharmaceutical composition, a vaccine and an immunogenic composition, comprising the recombinant replication-competent or replication-deficient negative-strand RNA virus of the invention. Further aspects of the invention relate to an RNA molecule, and a DNA molecule encoding the recombinant replication-competent or replication-deficient negative-strand RNA virus of the invention. Another aspect relates to a nucleocapsid, comprising the RNA molecule of the invention.

### BACKGROUND

History teaches that it is often only second-generation vaccines that bring a breakthrough in the complete control of permanently circulating viral infections: even in the fight against polio (poliomyelitis) 70 years ago, sustained success was only achieved with a second-generation vaccine that specifically mimics the natural enteral-mucosal route of infection. A previously developed first-generation vaccine, which at that time could only be injected into the upper arm muscle, did not result in “sterilizing immunity”, because the enteral-mucosal route of entry of poliovirus could not be protected against infection with poliovirus replicating in the digestive tract. Vaccinated individuals could thus continue to become infected with poliovirus and pass the disease onto others via enterally produced progeny viruses via enteral excretions (stool). The first-generation vaccine thus failed to establish sufficient herd immunity.

An overview of COVID-19 vaccines and candidate vaccines can be found on the WHO homepage (“COVID-19 vaccine tracker and landscape”: https://www.who.int/publications/m/item/draft-landscape-of-covid-19-candidate-vaccines). All previously approved anti-SARS-COV-2 vaccines belong to the first-generation of vaccines, according to a WHO definition. They are administered by i.m. injection, usually into an upper arm muscle. They do not enter the body by the respiratory tract, as in infection with SARS-COV-2. First-generation SARS-COV-2 vaccines elicit an immune response sufficient to mostly prevent severe courses of COVID-19, but do not achieve sufficient immunity of the respiratory tract.

This means that vaccinated subjects are still prone to be infected (the so-called breakthrough infections, being frequently observed). They may infect non-vaccinated persons by droplet infection: this means that all SARS-COV-2 vaccines approved so far, belonging to the first generation, are not able to effectively interrupt chains of infection. There is thus a great demand for a SARS-COV-2 vaccine which elicits a “sterilizing immunity” in the respiratory tract against COVID-19.

Breakthrough infections frequently occur and may overload the hospitals. The urgently needed herd immunity is a long way off. The return to “normal” lives as they existed before the pandemic does not succeed, with its implications for all areas of our lives.

The known vector-based (adenovirus-based) vaccines do not allow an integration of the SARS-CoV-2 Spike(S) protein into the envelope of the vector vaccine particles. In contrast to corona viruses, adenovirus-based vaccines do not immediately provide the recombinant S protein as antigen. Rather, after i.m. injection, the S protein must be produced in muscle cells infected by the vector vaccine, resulting in a delay of immune response and thus a loss of efficiency, in particular in development of mucosal immunity in the respiratory tract. The same applies to mRNA-based vaccines, administered i.m.

Repeated administration of an anti-SARS-COV-2 vaccine is mandatory if the immunological protection does not last long enough. At present, three or more vaccinations are considered to be necessary, for example a booster immunization every 6 months. Furthermore, newly emerging virus variants require administration of an adapted vaccine, and thus repeated administration. In the case of a vectored vaccine, the adenovirus capsid has a strong immunogenic potential, leading to a pronounced anti-vector immunity (anti-adenovirus immunity). Upon re-administration of an adenovirus vector, such anti-vector immunity may neutralize the vector, leading to a reduced efficacy and limited options of administering the first-generation vector vaccines in a schedule requiring two or more vaccinations. A pronounced anti-vector immunity would neutralize the effect of a booster immunization, which may thus become ineffective, or may require larger doses, resulting in increased effort in production and costs and risks by side-effects.

There is thus a need for SARS-COV-2 vaccine, which at least partially overcomes the disadvantages of first-generation vaccines described above.

Paramyxoviruses, in particular the Sendai virus, are enveloped viruses with a helical nucleocapsid. The envelope comprises a lipid membrane, which is derived from the plasma membrane of the host cell from which the virus was released. Transmembrane glycoproteins, namely the fusion protein (F) and hemagglutinin-neuraminidase (HN), are anchored in the viral envelope. The matrix protein (M) is located at the inside of the membrane. The nucleocapsid, i.e. the viral replication complex, consists of single-stranded RNA complexed with nucleoprotein (N), with in each case 6 nucleotides of the RNA bound by one N protein, an RNA-dependent RNA polymerase (L) and the cofactor phosphoprotein (P), forming the RNA polymerase complex. During primary transcription, the genome is transcribed by the viral RNA dependent RNA polymerase (vRdRp), and de novo protein synthesis begins. As soon as sufficient amounts of N proteins have been made, the vRdRp is thought to switch to a replicative mode to synthesize antigenomes. From that template, new genomes are replicated that are concomitantly encapsidated with N proteins.

The genomic RNA of virus families, i.e. negative-stranded RNA virus families, which is not immediately translated after infection of cells and release of this RNA into the cell, is usually forming a complex with a protein called the nucleoprotein (N protein or NP). Only this specific complex of viral RNA plus N proteins is recognized by the viral polymerase as a template for either viral genome replication or viral transcription. During viral genome replication newly synthesized genomes or antigenomes are immediately complexed with N proteins. During viral transcription, mRNA are synthesized that are not complexed with N proteins—this would prevent translation at the ribosomes. So, only viral genomes/antigenomes are complexed with N proteins. In the process of complexing viral RNA with N proteins, another viral protein is involved, the P protein. The P protein itself is forming a complex with the N protein; only this N-P complex enables complexing of the viral RNA with N proteins. If this complex between N and P cannot be formed, e.g. due to mutation of interacting sites on the N or the P protein, then no complexing of newly synthesized viral genomes/antigenomes can take place. No functional novel viral genomes can be generated and, therefore, no new functional viral particles can be generated. The virus would be replication-deficient. This replication-deficiency can be achieved through mutating the binding site of the P protein to the N protein, specifically through deletion of the nucleotides encoding for the amino acids 2-77 of the P protein. (see also Wiegand et al., 2007).

The negative-strand RNA genome of the Sendai virus contains the genes of the 6 structural proteins in the order: 3′-N-P-M-F-HN-L-5′. The P gene codes for a total of 8 proteins, the structural phosphoprotein and all non-structural proteins known to date.

The proteins P, N and L are important for functional transcription and replication (Lamb et al., Paramyxoviridae: The Viruses and their Replication. Fields Virology, 4th edition (2001), Lippincott, Williams & Wilkins, Philadelphia, 1305-1340).

WO 2006/084746 discloses a replication-defective and transcription-competent Sendai virus (SeV), which were used for the expression of transgenes.

Wiegand (Journal of Virology 2017: 91 (10), e02298-16) demonstrated that the ectodomain of the SeV surface protein F could be substituted by the corresponding ectodomain of the RSV virus F. Both SeV and RSV belong the family of paramyxoviruses. The substitution of the SeV F protein with the RSV F protein is an “intrafamiliar” exchange of functionally and structurally equivalent sequences.

Up to now no studies are available demonstrating an “interfamiliar” substitution of such functionally essential proteins between the completely different virus families of paramyxoviruses and coronaviruses, without a loss of function.

Attenuation of virus replication refers to suboptimal conditions of a specific vector construct compared to its natural/wildtype counterpart. While the latter has adapted during evolution to its preferred host organism or cell type and, thus, achieves high viral reproduction rates, do attenuated viral vectors only generate suboptimal titers during replication. There are many different potential reasons how attenuation and suboptimal replication can happen. Attenuation can come from the viral vector itself, from its host system, from the conditions during replication or from a mixture of these. Of course, changes to the viral genome can have a huge impact on the viral replication behaviour as (an) introduced mutation(s) most likely do not represent any optimization that the virus would be selected for by nature. The effect of single or multiple point mutations can usually already be clearly recognized depending on the affected gene or gene product. Mutations that affect the functionality of viral enzymes involved in viral replication can have a large impact on virus reproduction. For example, an N-terminal deletion in the Sendai virus P gene can lead to replication deficiency, as the L-Protein cannot interact with its co-factor P protein in a way that is needed to interact with the viral genome and mediate the synthesis of copies of the viral RNA genome. Another type of mutation can affect structural proteins of a virus, such as surface or envelope proteins. Envelope proteins are needed during virus reproduction to form new viral particles as release these particles from the host cell. Further, these proteins are essential to viral interaction with a new host cell and the following uptake into the cell through receptor binding and mediation of the uptake mechanism e.g. by membrane fusion or vesicular uptake into the cell. Different viruses follow different uptake strategies. For example, the Sendai virus HN and F proteins in the membranous envelope of the viral particles enable receptor binding followed by fusion of the viral membrane with the host cell membrane, thereby releasing the viral genome into the host cell. The SARS-CoV-2 rather enters the host cell through vesicular uptake mediated by its surface protein S. Large mutations of these surface/envelope proteins such as point mutations of essential amino acids or even exchange of entire envelope proteins between genomes from different virus families can have a huge impact on the normal viral life cycle and its reproduction efficacy.

### DETAILED DESCRIPTION

The inventors developed a second-generation vaccine (in particular against SARS-COV-2) fulfilling the following requirements:
the vaccine vector is safe with maximum attenuation, so that it can be used in healthy subjects and also in pre-diseased, partly immune-suppressed subjects,
the vector should efficiently penetrate the mucus layer on the mucosa and thus be suitable as a vaccine vector for induction of mucosal immunity in the respiratory tract,
the vaccine vector is to be administered by the natural infection route (in particular in the anti-SARS-COV-2 vaccine: the mucosal route in the respiratory tract),
the vaccine vector elicits immediate mucosal immunity, by presentation of an antigen on the viral surface (in particular in the anti-SARS-COV-2 vaccine: for example, the Spike(S) protein),
the vaccine vector elicits no, or at most a reduced anti-vector immunity, to prevent loss of efficacy by repeated administration,
after expression of the transgene in infected cells the vector elicits a systemic immune response.

The inventors developed a vector-based vaccine in accordance with the above explanations, carrying the SARS-COV-2 Spike protein on the surface of the vector vaccine particles. By this design, the protein is presented directly to the immune system in the course of intra-nasal/mucosal application, just as in natural infection of the respiratory tract by SARS-COV-2. Furthermore, upon entry of mucosal cells of the host, the virus is capable of producing the SARS-COV-2 Spike protein, and displaying it on the cell surface. In this way, an extensive protection, in particular including “sterilizing immunity”, against the extremely threatening COVID-19 disease is achieved and ensured.

The inventors found that a suitable vector is an attenuated (replication deficient) Sendai virus (SeV). The Sendai virus (family Paramyxoviruses) typically infects rodents and causes a highly transmissible respiratory tract infection.

The Sendai virus (SeV, paramyxovirus family) carries two proteins on the surface: (i) the hemagglutinin-neuraminidase (HN) protein, and (ii) the fusion (F) protein. The HN protein mediates binding (adsorption) of the SeV virus particle at the cellular receptor. The F protein mediates fusion of the SeV virus membrane with the cytoplasmic membrane. After membrane fusion (penetration), the SeV replication complex (nucleocapsid) is released into the cell.

The biologically active forms of both HN and F present on the SeV surface are oligomers. Upon virus maturation, these oligomers are concentrated in specific regions of the cytoplasmic membrane by interaction with the SeV Matrix protein (M), being a major prerequisite for release of newly formed virus particles from the infected cell (“budding”).

At the beginning of the inventor's work, it was unknown if a recombinant protein obtained from other virus families (e.g. the S protein of SARS-COV-2, a coronavirus family member) could be integrated in the envelope of a SeV vaccine vector (paramyxovirus family) without a significant negative impact on the SeV HN and F protein function (adsorption and penetration) in the infection of target cells.

It was also completely unknown if and which modification of a recombinant protein (e.g. a chimeric SARS-COV-2 S protein) would be required for successful integration in the SeV envelope and display on the surface, and which modification will be tolerated, such that the biological function of the recombinant protein remains unaffected, for example antigenicity of the SARS-COV-2 and functionality during infection process.

The only polypeptide on the surface of SARS-COV-2 is the spike protein S. The S protein is present in oligomeric form. The S protein is the largest viral protein with fusogenic function (more than 1200 amino acid residues in length). Approximately 30-60 S protein molecules are anchored in the envelope of a single SARS-COV-2 particle, with an average distance of 15 nm. At the beginning of the inventor's work, it was unknown if the distance of the S proteins on the surface is essential to the function, or if the S protein, when recombinantly integrated in the envelope, would be functional in combination with SeV F and HN, or if combined display on the SeV surface resulted in partial or complete loss of function.

Furthermore, is was unknown if a recombinant SARS-COV-2 S protein could supplement the function of the F and/or HN polypeptide if sequences encoding the F and/or HN polypeptides are deleted partially or completely from the SeV genome.

These aspects make it clear that design and construction of a SeV vector-based vaccine against SARS-COV-2 could in no way be considered trivial or simple, but required considerable inventive activity.

The inventors constructed a recombinant Sendai virus displaying a recombinant antigen obtained from SARS-COV-2 (e.g. SARS-COV-2 Spike protein) on its surface. Examples 1 and 2 describe cDNA constructs of recombinant Sendai virus (SeV), capable of expressing the coronavirus SARS-COV-2 Spike(S) protein. The S protein can be expressed as a chimeric protein, comprising the ectodomain of the S protein, the cytoplasmic domain of an SeV polypeptide and the transmembrane domain of an SeV polypeptide or SARS-COV-2 Spike. The inventors showed that a candidate vaccine, provided as a cDNA could be rescued from GMP qualified Vero cells and propagated in V3-10 helper cells derived from Vero cells. The inventors showed that the SARS-COV-2 S protein was integrated in the candidate virus particles.

The inventors found that a recombinant SARS-COV-2 S protein is displayed in a functional form on the SeV surface. The inventors provided a SeV vaccine vector (i) with a reduced amount of surface F and/or HN protein, or (ii) with no F and HN protein, thus inducing at most a small or no anti-vector immunity. The inventors recognized that the S protein of coronaviruses (positive-stranded) is capable of complementing the adsorption and penetration capability in negative-stranded RNA viruses if the native (endogenous) genes mediating these capabilities are deleted in these viruses. In particular the adsorption and penetration capability of the negative-stranded paramyxovirus F and HN proteins could be complemented by the S protein from a virus of the positive-stranded coronavirus family, having adsorption and penetration capabilities. This is the first time that functional “interfamiliar” substitution of two endogenous F and HN proteins in a paramyxovirus (SeV) by a single coronavirus protein could be demonstrated.

The inventors found that attenuation of a Sendai virus can be achieved by modification of the P protein, resulting in a replication-deficient virus.

The inventors also found that attenuation can be achieved in a replication-competent SeV, wherein the surface proteins F and/or HN are at least partially replaced by SARS-COV-2 surface protein S, or a fragment thereof, so that the S protein of fragment thereof can exert the function of the F and/or HN protein. The inventor also found that such modified replication-competent SeV has an increased production efficacy, compared with replication-deficient SeV of the invention (Example 4).

The inventors also found that intranasal immunization was able to stimulate a specific mucosal immune response against SARS-COV-2. Immunization with a virus of the invention dose-dependently induced specific IgA antibodies (Example 5).

If a vaccinated subject has already acquired immunity against SARS-COV-2 in the respiratory tract, for example by SARS-COV-2 infection, the immune system of the subject may recognize the recombinant replication-competent or replication-deficient negative-strand RNA virus vaccine vector, in particular the SeV vaccine vector against SARS-COV-2, and the vaccine can act as a booster immunization. If a vaccinated subject has not yet acquired immunity against SARS-COV-2 in the respiratory tract, the vaccine vector can induce immunity against SARS-COV-2 via different immune-stimulatory pathways and interactions with the immune system. First, the vaccine vector particles per se are immunogenic for SARS-COV-2 as they display the Spike protein on the surface; in this way they vaccine vector can function as a e.g. nanoparticle vaccine or Virus-like Particle vaccine (VLP). These particles are often recognized by antigen presenting cells of the immune system, e.g. dendritic cells, or are taken up by macrophages, which then cross present parts of the Spike protein to T lymphocytes, which can then differentiate into helper and effector cells. Secondly, the vaccine vector can infect cells, e.g. of the respiratory tract, and express the encoded Spike protein inside of these cells. This will then result in the expression of the Spike protein on the surface of these cells, which can then be recognized by the immune system as infected and will also result in the display of peptides of the Spike protein on MHC-I molecules after standard protein degradation inside of these cells.

In view of the experimental evidence obtained with a paramyxovirus (e.g. SeV) recombinantly displaying an antigen (e.g. SARS-COV-2 S protein) on the surface, and at the same time, eliciting at most a small or no anti-vector immunity, the inventors concluded that a recombinant negative strand RNA virus vector could be constructed eliciting small or no anti-vector immunity and carrying a recombinant antigen.

In a first aspect, the present invention relates to a recombinant replication-deficient negative-strand RNA virus, comprising in its genome:
(a) a nucleotide sequence encoding a P polypeptide, said nucleotide sequence being modified in comparison to the wildtype form, said modification leading to replication deficiency of the negative-strand RNA virus, and
(b) at least one nucleotide sequence encoding at least one heterologous polypeptide, wherein the at least one heterologous polypeptide is displayed on the surface of the virus particle.

In the present invention, the term “virus” relates to an infectious agent replicating only inside of living cells. A virus has no own metabolism. The term “virus”, as used herein includes all forms inside a host cell, and virus particles (also termed virions) outside the host cell, i.e. released from the host cell and being capable of infecting new host cells. An RNA virus may be a single-stranded or double-stranded RNA virus.

In the present invention, the term “genome” or “viral genome” are used interchangeably and refer to the entirety of nucleic acids present in the nucleocapsid. The genome may be unsegmented (only one nucleic acid molecule) or segmented (two or more nucleic acid molecules). A single-stranded virus may be a positive-stranded virus or a negative-stranded virus. In negative-stranded viruses, the nucleic acid complementary to the genome is termed antigenome. In a negative-stranded virus, the genes are transcribed into mRNA, which are translated into polypeptides. In a positive-stranded virus, the nucleic acid complementary to the genome is termed genome. In a positive-stranded virus, the genomic sequences can be directly translated into polypeptides. In the present invention, the genome may be a recombinant genome. The recombinant replication-deficient negative-strand RNA virus, as described herein, may comprise a recombinant genome. The recombinant genome may comprise a recombinant RNA molecule

In the present invention, the terms “interfamiliar” and “intrafamiliar” describe a relation among viruses of different virus families or the same virus family. For example, an interfamiliar exchange of nucleotide sequences is an exchange among viruses of different families, and an intrafamiliar exchange of nucleotide sequences is an exchange among viruses of the same family.

As used herein, the term “heterologous” or “exogenous” refers to a polypeptide and/or nucleic acid that is foreign to a particular virus, such as a negative-strand RNA virus. The heterologous (exogenous) polypeptide and/or nucleic acid is not naturally present in a particular virus and may be introduced to the viral genome by artificial or recombinant means. For example, a heterologous (exogenous) nucleotide sequence is a foreign sequence introduced into a viral genome. The heterologous (exogenous) nucleotide sequence may be in operative linkage with a native or endogenous viral sequence, such as an expression control sequence. The heterologous (exogenous) nucleotide sequence may also be inserted in frame is a native or endogenous viral coding sequence, such that a fusion protein or chimeric protein is expressed, comprising the heterologous (exogenous) polypeptide sequence and a native or endogenous amino acid sequence.

Yet another aspect of the present invention relates to a recombinant replication-competent negative-strand RNA virus, comprising in its genome:
(a) a nucleotide sequence encoding a P polypeptide, wherein the P protein exerts in particular its natural function in the viral replication cycle,
(b) at least one nucleotide sequence encoding at least one heterologous polypeptide, wherein the at least one heterologous polypeptide is displayed on the surface of the virus particle,
   wherein the heterologous polypeptide comprises a viral antigen, and wherein the viral antigen and the negative-stranded RNA virus are selected from viruses of different families.

In particular, the recombinant replication-competent negative-strand RNA virus is attenuated. In the recombinant replication-competent negative-strand RNA virus, the P protein can be a wildtype P protein, for example a P protein encoded by SEQ ID NO: 1 or a sequence with at least 80%, at least 85%, at least 90%, at least 95%, or at least 98% identity thereto. The wildtype P protein may have an amino acid sequence comprising SEQ ID NO: 2, or an amino acid sequence with at least 80%, at least 85%, at least 90%, at least 95%, or at least 98% identity thereto.

In the present invention, the term “attenuated virus” relates to a virus with reduced or no pathogenicity, in particular mostly due to a reduced or missing replication. The attenuated virus may be capable of eliciting an immune response without producing the specific disease caused by the non-attenuated form of the virus.

In particular, the present invention also relates to a negative-strand RNA virus which is replication-deficient. Loss of the capacity for replication means that in a target cell which does not produce in trans any of the functions deleted in the virus, no detectable virus genome replication is found, and in contrast to a reduced or conditional replication deficiency, also no permissive conditions exist, in which replication can occur. As used herein, the negative-strand RNA virus of the invention may still be infectious, i.e. is capable of adsorbing to a host cell and penetrating into that host cell. As used herein, the negative-strand RNA virus that is infectious may also direct expression of at least the heterologous gene in the host cell.

As used herein, a “recombinant replication-competent or replication-deficient negative-strand RNA virus according to the invention” describes two different viruses of the present invention: (i) a recombinant replication-competent negative-strand RNA virus, and (ii) a recombinant replication-deficient negative-strand RNA virus. The term “recombinant replication-competent or replication-deficient negative-strand RNA virus according to the invention”, or a similar wording (for example, relating to specific virus species) is used in this specification in particular to describe features which can be independently present in (i) the recombinant replication-competent negative-strand RNA virus, and (ii) the recombinant replication-deficient negative-strand RNA virus. Embodiments of the invention relating to the recombinant replication-deficient negative-strand RNA virus are in particular described in Items 1-69. Embodiments of the invention relating to the recombinant replication-competent negative-strand RNA virus are in particular described in Items 70-133.

The recombinant virus according to the invention may be transcription-competent, i.e. the virus may be capable of inducing viral and heterologous mRNA synthesis and translation into viral polypeptides and heterologous polypeptides. In particular, gene products encoded by the virus are transcribed after infection in a target cell, so that expression of the viral proteins including one or more heterologous gene products can take place in the target cell. A heterologous polypeptide may be displayed on the surface of a target cell, i.e. a mucosal cell of a vaccinated subject, and thereby induce and/or improve the immune response to the heterologous polypeptide

Transcription may include primary transcription and secondary transcription. As used herein, primary transcription starts from the minimal replication unit, i.e. a nucleocapsid penetrated into the cell, said nucleocapsid comprising a single-stranded RNA molecule complexed with nucleoprotein (N), an RNA-dependent RNA polymerase (L) and the phosphoprotein (P), forming the RNA polymerase complex. Primary transcription may also start from a DNA molecule encoding the virus, in particular a cDNA molecule. During primary transcription, the genome is transcribed by the RNA polymerase, and de novo protein synthesis begins. As soon as new genomes are replicated, these templates also can be transcribed. This phase of transcription is called secondary transcription and is much more effective than primary transcription.

In the present invention, the term “gene product” describes a product obtained by expression of a gene. A gene product includes a nucleic acid transcribed from the gene (e.g. mRNA), and a polypeptide or protein obtained by translation from an mRNA.

The recombinant replication-deficient RNA virus according to the invention may be capable of being replicated in a host cell trans-complemented with exogenous sequences encoding the deficient viral proteins (“helper cells”). As a result, replication-deficient virus particles are obtained. The eukaryotic host cell may be co-transfected with at least one eukaryotic expression vector encoding viral proteins required for formation of viral nucleocapsid, i.e. structural proteins and a polymerase required for replication of the cDNA sequences. For example, a host cell may be transfected with a plasmid comprising a replication-deficient viral genome, said genome being modified in comparison to the wildtype form, said modification leading to replication deficiency of the negative-strand RNA virus, and the host cell may be co-transfected with three plasmids, encoding native viral proteins N (capsid), L and P (forming an RNA-dependent RNA polymerase complex), respectively. The genome may be transcribed to an antigenomic RNA by a polymerase endogenous to the host cell (e.g. RNA Pol II). The antigenome may be assembled into a nucleocapsid. FIG. 1 describes an exemplary rescue strategy of a replication deficient Sendai virus.

For initial production of the replication-deficient negative strand RNA virus (referred to herein as “rescue” or “viral rescue”), a eukaryotic host cell may be transfected with the cDNA comprising the viral genome. In particular, the host cell capable of viral rescue from a DNA molecule coding the viral genome does not require trans-complementation with exogenous sequences encoding the deficient viral proteins.

In the present invention, the term “P polypeptide” refers to the Phosphoprotein present in negative stranded RNA viruses. P polypeptide is a cofactor of the RNA-dependent RNA polymerase of these viruses and has a role in viral transcription and replication.

In the present invention, the term “wild-type” describes a recombinant negative-strand RNA virus comprising essentially no recombinant sequences or modification, such as deletion. The term “wildtype” with respect to a particular sequence relates to a sequence comprising essentially no recombinant sequences or modification, such as deletion. For example, the wildtype sequence of the P polypeptide is a sequence of the P polypeptide comprising essentially no recombinant sequences or modification, such as deletion. If “wildtype” refers to a specific sequence of the recombinant negative-strand RNA virus (such as the P sequence), other sequences may be modified, or may also be unmodified wildtype sequences. The skilled person knows wildtype P polypeptide sequences.

The recombinant replication-competent or replication-deficient negative-strand RNA virus according to the invention can be based on a naturally occurring negative-strand RNA virus. The recombinant replication-competent or replication-deficient negative-strand RNA virus according to the invention can be selected from the order of Mononegavirales. Preferred families on which the recombinant replication-competent or replication-deficient negative-strand RNA virus according to the invention can be based are Artoviridae, Bornaviridae, Filoviridae, Lispiviridae, Mymonaviridae, Nyamiviridae, Paramyxoviridae, Pneumoviridae, Rhabdoviridae, Sunviridae, and Xinmoviridae. Preferred families on which the recombinant replication-competent or replication-deficient negative-strand RNA virus according to the invention can be based are Paramyxoviridae (paramyxoviruses), Rhabdoviridae (rhabdoviruses), Filoviridae, Bornaviridae, and recombinant variants thereof.

In the present invention, single-stranded non-segmented negative strand RNA viruses include Paramyxoviridae (paramyxoviruses), Rhabdoviridae (rhabdoviruses), Filoviridae (filoviridae) and Bornaviridae (bornaviruses).

Examples of paramyxoviruses include, but are not limited to the Sendai virus, human and bovine parainfluenza virus, e.g. human parainfluenza virus (hPIV) type 1, 2, 3, 4a or 4b, Newcastle disease virus (NDV), mumps virus, measles virus, respiratory syncytial virus (RSV) and human respiratory syncytial virus (hRSV). Examples of rhabdoviruses include, but are not limited to, the vesicular stomatitis virus (VSV).

Preferably, the recombinant replication-competent or replication-deficient negative-strand RNA virus may be a paramyxovirus. Preferably the recombinant replication-competent or replication-deficient negative-strand RNA virus of the present invention may be a Sendai virus, e.g. the Fushimi strain (ATCC VR105). Recombinant variants of the aforementioned viruses, as described for example in EP-A-702 085, EP-A-0 863 202 or WO 01/42445, are also covered by the invention.

In the present invention, single-stranded positive strand viruses include the family of coronaviruses (Coronaviridae). Examples of coronaviruses include MERS-COV (Middle East Respiratory Syndrome Coronavirus), SARS-CoV (Severe acute respiratory syndrome-related coronavirus, also termed SARS-COV-1), and SARS-COV-2 (severe acute respiratory syndrome coronavirus type 2).

In the present invention, the term “nucleocapsid” relates to a complex comprising the viral nucleic acid and at least one viral protein (the “capsid”). The nucleocapsid can be present in the host cell, as well as in the virus particle (virion).

In the present invention, the term “viral envelope” relates to the outer layer of the virus, if present. The envelope can be derived from host cell membranes and comprises viral peptides, for example peptides capable of adsorbing to a host cell receptor and mediating penetration into the cell. These peptides can be anchored in the envelope by a transmembrane domain and an intracellular domain. In the present invention, the envelope of the recombinant replication-competent or replication-deficient negative-strand RNA can comprise the at least one heterologous polypeptide, for example the SARS-CoV-2 Spike protein or fragment thereof, so that it is displayed on the surface of the virus particle.

In the present invention, the terms “peptide” and “polypeptide” can be used interchangeably.

In the present invention, the terms “nucleic acid” and “nucleic acid molecule” and “polynucleotide” can be used interchangeably.

In the present invention, the term “viral peptide” relates to a peptide required for nucleic acid synthesis (transcription and replication, e.g. a polymerase or a polymerase complex), envelope structure, if an envelope is present, nucleocapsid structure, and surface structure (required for adsorption on and penetration into a host cell). No uniform terminology of viral peptides and genes encoding such peptides exists. In the present invention, a reference to a specific gene and/or peptide in a particular virus or virus family is intended to include a reference to homologous genes and/or peptides in other virus species and/or families, in particular genes and/or peptides having the same function (also termed herein “functional homologue”). For example, a reference to the P peptide, specifically described in the Sendai virus herein, includes a reference to any one of peptides described in single-stranded negative-strand RNA viruses having essentially the same function as the Sendai virus P peptide.

In the present invention, the nucleotide sequence (a) encoding a P polypeptide (also termed P protein or phosphoprotein) can be modified in comparison to the wildtype form (i.e. wildtype P sequence), said modification leading to replication deficiency of the negative-strand RNA virus. As used herein, the term “P polypeptide” describes polypeptides of negative-strand single-stranded RNA viruses, having essentially the same function as the P polypeptide of paramyxoviruses, in particular the Sendai virus. Reference is made herein to the Sendai virus sequences encoding the P polypeptide, as an exemplary, non-limiting example of a P polypeptide (SEQ ID NO:2) and a sequence encoding a P polypeptide (SEQ ID NO:1).

As used herein, “modification” includes a deletion, insertion and/or substitution in a nucleotide sequence or polynucleotide. If the nucleotide sequence comprises a coding sequence, such modification may result in a modified polypeptide.

In the nucleotide sequence (a) encoding the P polypeptide, the modification in the nucleotide sequence (a) may be located in the nucleotide sequence encoding the N terminus of the P polypeptide. At least the region of amino acids 33-41 of the P polypeptide may be modified, which are important for the capacity for replication. Modification in the region of amino acids 2-77 in the P polypeptide leads to loss of capacity for replication, for example a deletion of (i) the amino acids 2-77 of the protein encoded by gene P or (ii) a partial sequence of (i) sufficient for loss of the capacity for replication. Corresponding mutations can also take place in P proteins of other negative-strand RNA viruses, e.g. of other paramyxoviruses, e.g. hPIV3.

In particular, the modification in the nucleotide sequence (a) is a deletion in the nucleotide sequence encoding the N terminus of the P polypeptide. More particular, the modification in the nucleotide sequence (a) is
(i) a deletion of the nucleotide sequence encoding the amino acids 2-77 of the P polypeptide (,,deltaP 2-77″, SEQ ID NO:4), in comparison to the wildtype form, or
(ii) a partial sequence of (i), leading to replication deficiency of the RNA virus.
SEQ ID NO:3 describes a nucleotide sequence encoding the SeV P Protein with deletion of amino acids 2-77 of SEQ ID NO:4.

In particular, the C-terminal region of the P polypeptide (starting from amino acid 320) does not have a modification impairing the transcription function.

In the replication-competent or replication-deficient negative-stranded RNA virus as described herein, the genome may further comprise
(c) a nucleotide sequence encoding a negative-strand RNA virus N polypeptide,
(d) a nucleotide sequence encoding a negative-strand RNA virus M polypeptide, and
(e) a nucleotide sequence encoding a negative-strand RNA virus L polypeptide.

As used herein, the term “N polypeptide”, “N protein” or “nucleoprotein” describes polypeptides of negative-strand single-stranded RNA viruses, having essentially the same function as the N polypeptide of paramyxoviruses, in particular the Sendai virus. The skilled person knows suitable N polypeptides of paramyxoviruses and the function thereof. In the nucleocapsid of single-stranded RNA viruses, the RNA molecule is complexed with the N polypeptide. As used herein, a reference to the N polypeptide is not limited to paramyxoviruses, and includes functional and structural homologues in other viral families.

As used herein, the term “M polypeptide”, “M protein” or “matrix protein” describes polypeptides of negative-strand single-stranded RNA viruses, having essentially the same function as the M polypeptide of paramyxoviruses, in particular the Sendai virus. The skilled person knows suitable M polypeptides of paramyxoviruses and the function thereof. The M polypeptide is located at the inner surface of the viral envelope. As used herein, a reference to the M polypeptide is not limited to paramyxoviruses, and includes functional and structural homologues in other viral families.

As used herein, the term “L polypeptide”, “L protein” or “large protein” describes polypeptides of negative-strand single-stranded RNA viruses, having essentially the same function as the L polypeptide of paramyxoviruses, in particular the Sendai virus. The skilled person knows suitable L polypeptides of paramyxoviruses and the function thereof. The L polypeptide is the largest protein of the polymerase complex and has RNA-dependent polymerase activity. As used herein, a reference to the L polypeptide is not limited to paramyxoviruses, and includes functional and structural homologues in other viral families.

Furthermore, the genome of the recombinant replication-competent or replication-deficient negative-strand RNA virus may further comprise
(f) a nucleotide sequence encoding a negative-strand RNA virus F polypeptide, and
(g) a nucleotide sequence encoding a negative-strand RNA virus HN polypeptide.

As used herein, the term “F polypeptide”, “F protein” or “Fusion protein” describes polypeptides of negative-strand single-stranded RNA viruses, having essentially the same function as the F polypeptide of paramyxoviruses, in particular the Sendai virus. The skilled person knows suitable F polypeptides of paramyxoviruses and the function thereof. The F polypeptide mediates fusion of the virus particle with the host cell membrane. As used herein, a reference to the F polypeptide is not limited to paramyxoviruses, and includes functional and structural homologues in other viral families.

As used herein, the F polypeptide or the sequence encoding the F polypeptide may comprise a sequence described herein:

SEQ ID NO:6 describes the amino acid sequence of the Sendai virus (SeV) SeV F polypeptide. SEQ ID NO: 5 describes a nucleotide sequence encoding the Sendai virus (SeV) SeV F polypeptide of SEQ ID NO:6

As used herein, the term “HN polypeptide”, “HN protein” or “Hemagglutinin-Neuraminidase” describes polypeptides of negative-strand single-stranded RNA viruses, having essentially the same function as the HN polypeptide of paramyxoviruses, in particular the Sendai virus. The skilled person knows suitable HN polypeptides of paramyxoviruses and the function thereof. The HN polypeptide mediates adsorption of the virus particle to the host cell. As used herein, a reference to the HN polypeptide is not limited to paramyxoviruses, and includes functional and structural homologues in other viral families.

As used herein, the HN polypeptide or the sequence encoding the HN polypeptide may comprise a sequence described herein:

SEQ ID NO: 12 describes the amino acid sequence of the Sendai virus (SeV) SeV HN polypeptide. SEQ ID NO:11 describes a nucleotide sequence encoding the Sendai virus (SeV) SeV HN polypeptide of SEQ ID NO:12.

As used herein, “at least one nucleotide sequence (b)” includes one, two, three, four or even more nucleotide sequences (b), each independently encoding a heterologous polypeptide. Preferably, “at least one nucleotide sequence (b)” includes one or two nucleotide sequences (b), more preferably one nucleotide sequence (b). “At least one nucleotide sequence (b)” and “a nucleotide sequence (b)” may be used herein interchangeably.

The at least one heterologous polypeptide encoded by nucleotide sequence (b) may comprise any polypeptide suitable for therapy, for example an antigen, against which an immune response is to be produced, or a therapeutic protein, e.g. a protein for virotherapy.

The at least one heterologous polypeptide encoded by nucleotide sequence (b) may comprise a heterologous antigen, e.g. originating from a pathogen (such as a virus, a bacterium, a fungus or a protozoon), a tumor antigen or an autoantigen. The skilled person knows antigens suitable to prepare an immunogenic composition and/or vaccine against a pathogen, such as a virus, a bacterium, a fungus or a protozoon. An Example of an antigen suitable for the preparation of an immunogenic composition and/or vaccine against SARS-COV-2 is the SARS-COV-2 spike protein (S protein), or an immunogenic fragment thereof.

The heterologous polypeptide may comprise a viral polypeptide, in particular a viral antigen. The viral polypeptide or the viral antigen and the negative-stranded RNA virus may be selected from viruses of different families. For example, the negative-stranded RNA virus may be a paramyxovirus, and the heterologous antigen may be selected from viral antigens of a family other than paramyxoviruses, for example selected from viral polypeptides and antigens of the families Rhabdoviridae, Filoviridae, Bornaviridae (negative strand viruses) and coronaviruses (positive stranded viruses).

The heterologous polypeptide may also comprise an antigen from a virus which is not a negative-strand RNA virus.

The heterologous polypeptide may also comprise an antigen from a positive-strand virus, such as a coronavirus.

It is preferred that the single-stranded negative-strand RNA virus is a paramyxovirus, such as a Sendai virus, and the heterologous polypeptide comprises an antigen from a positive-strand virus, such as a coronavirus, in particular SARS-COV-2.

It is more preferred that the single-stranded negative-strand RNA virus is a Sendai virus, and the heterologous polypeptide is an antigen from a coronavirus.

It is more preferred that the single-stranded negative-strand RNA virus is a paramyxovirus, such as a Sendai virus, and the heterologous polypeptide comprises a Spike protein (S protein) or an immunogenic fragment thereof, from a coronavirus, in particular SARS-COV-2.

The antigen may be a surface polypeptide of a virus, or an immunogenic fragment thereof. The skilled person knows suitable antigens, for example a polypeptide present on a virus particle. An example of a viral surface polypeptide is the SARS-COV-2 spike protein (S protein), or an immunogenic fragment thereof.

The at least one nucleotide sequence (b) may be located in the RNA molecule between two coding sequences, at a position upstream the 5′ terminal coding sequence and/or downstream the 3′ terminal coding sequence. For example, in a paramyxovirus, the at least one nucleotide sequence (b) may be located independently upstream the 5′ end of the N gene, between the genes N and P, P and M, M and F, F and HN, and HN and L, and/or downstream the 3′ end of the L gene. By this arrangement, the least one nucleotide sequence (b) present in the RNA molecule does not interfere with the endogenous viral sequences and expression thereof.

In the recombinant replication-competent or replication-deficient RNA virus of the present invention, the genome may further comprise
(f) a nucleotide sequence encoding a negative-strand RNA virus F polypeptide, and
(g) a nucleotide sequence encoding a negative-strand RNA virus HN polypeptide,
and the at least one nucleotide sequence (b) may be located in the RNA molecule independently between two coding sequences, at a position upstream the 5′ terminal coding sequence and/or downstream the 3′ terminal coding sequence for example upstream the 5′ end of the N gene, between the genes N and P, P and M, M and F, F and HN, and HN and L, and/or downstream the 3′ end of the L gene. Examples of such constructs are described in the Examples (“Type A” constructs). Preferably, the at least one nucleotide sequence (b) is located between the P and the M gene.

In the replication-competent or replication-deficient negative-strand RNA virus, the at least one nucleotide sequence (b) may be inserted in or replaces at least partially at least one native (endogenous) sequence encoding at least one viral polypeptide, such that the function or activity of the at least one endogenous viral polypeptide is disrupted at least partially or completely. In particular, the at least one viral polypeptide is different from the P polypeptide. The viral polypeptide may be N, M, F, HN and/or L of paramyxoviruses, and homologues thereof in other virus families. Preferably, the at least one nucleotide sequence (b) may be inserted in or replaces at least partially F and/or HN of paramyxoviruses, and homologues thereof in other virus families, such that the function or activity of the F and/or HN is disrupted at least partially or completely.

As used herein, the term “native” or “endogenous” refers to a polypeptide and/or nucleic acid that is naturally present in a particular virus, in particular a negative-strand RNA virus.

The skilled person understands that a construct, as described herein, may be obtained by various ways from a wild-type or modified negative-strand single-stranded RNA virus. For example, a substitution modification can be obtained by a single recombination event, or by a deletion and subsequence insertion, or by insertion and subsequent deletion. In the present invention, these modifications can be equivalent.

A heterologous sequence, inserted into the replication-competent or replication-deficient negative-strand RNA virus without modification of viral genes and their function, can be a sequence which is not essential for propagation and/or infection. There is thus a considerable risk that such non-essential heterologous sequence is lost during virus propagation, e.g. by an incorrect transcription by the RNA polymerase. For example, during nucleocapsid formation, in each case 6 nucleotides of the RNA may be bound by one N protein. Incorrect transcription may lead to an RNA molecule the length of which cannot be divided by 6, and thus nucleocapsid formation fails.

In the present invention, the replication-competent or replication-deficient negative-strand RNA virus may comprise a genetically stable RNA molecule. As used herein, “genetically stable” relates to nucleic acid molecules or genomes which can be replicated and/or propagated without alterations of the sequence during the replication and/or propagation process. In particular, a nucleic acid molecule is genetically stable if essentially all coding sequences are essential to replication and/or propagation, i.e. essentially all coding sequences are required for replication and/or propagation. Any alteration in sequence may prevent replication and/or propagation of the altered molecule, as it may lack an essential sequence.

In the present invention, the nucleotide sequence (b) encoding the at least one heterologous polypeptide may be essential to viral replication and/or propagation. The at least one nucleotide sequence (b) encoding the at least one heterologous polypeptide may provide the function or activity of at least one native viral polypeptide. In particular, nucleotide sequence (b) exhibits essentially the same function as at least one endogenous viral sequence. In the present invention, the function or activity of at least one endogenous viral sequence may be at least partially or completely disrupted, and the heterologous polypeptide encoded by sequence (b) may be functionally expressed. For example, the sequences coding such native viral polypeptide(s) may be deleted at least partially or completely. The heterologous nucleotide sequence may substitute the function of the at least one native sequence, and may thus become essential to viral replication and propagation and may provide genetic stability of the construct. In particular, the heterologous polypeptide encoded by sequence (b) is displayed on the surface in biologically active form. In the present invention, for example the SARS-COV-2 S protein exhibits essentially the same function as the sequences encoding SeV F and HN.

In the recombinant replication-competent or replication-deficient RNA virus of the present invention, an endogenous sequence encoding the F polypeptide and/or an endogenous sequence encoding the HN polypeptide may be modified, such that their function or activity is at least partially or completely disrupted. The nucleotide sequence (b) may be inserted in the locus of the F and/or HN gene. Preferred is that nucleotide sequence (b) is inserted in the locus of the HN gene. Also preferred is that nucleotide sequence (b) is inserted in the locus of the F gene.

In the native SeV virus, the F and HN are directly adjacent. In particular, the sequence encoding the F polypeptide and/or a sequence encoding the HN polypeptide may deleted at least partially or completely. Examples of such constructs are described in the Examples (“Type B” and “Type C” constructs).

For example, the nucleotide sequence (b) may replace at least one native (endogenous) sequence of the viral genome at least partially or completely. For example, a sequence (b), encoding the SARS-CoV-2 S protein may replace at least partially endogenous sequences encoding SeV F and/or HN.

For example, a sequence (b), encoding the ectodomain of SARS-COV-2 S protein may be inserted in sequences encoding SeV F and/or HN, such that the ectodomain of SARS-COV-2 S protein may be fused in frame with F transmembrane and/or cytoplasmic domain, and/or the HN transmembrane and/or cytoplasmic domain, wherein the function and/or activity of native HN and/or F polypeptides are at least partially or completely disrupted.

As used herein, the transmembrane domain, the cytoplasmic domain of the F and HN polypeptide, or a sequence encoding such domain may independently comprise a sequence described herein:
SEQ ID NO: 8 describes the amino acid sequence of the SeV F protein transmembrane domain (partial sequence of SeV F protein of SEQ ID NO:6). SEQ ID NO: 7 describes a nucleotide sequence encoding the SeV F protein transmembrane domain, encoding SEQ ID NO: 8.
SEQ ID NO:10 describes the amino acid sequence of the SeV F protein cytoplasmic domain (partial sequence of SeV F protein of SEQ ID NO:6). SEQ ID NO: 9 describes a nucleotide sequence encoding the SeV F protein cytoplasmic domain, encoding SEQ ID NO:10.
SEQ ID NO:16 describes the amino acid sequence of the SeV HN protein transmembrane domain (partial sequence of SeV HN protein of SEQ ID NO: 12). SEQ ID NO:15 describes a nucleotide sequence encoding the SeV HN protein transmembrane domain, encoding SEQ ID NO:16.
SEQ ID NO:14 describes the amino acid sequence of the SeV HN protein cytoplasmic domain (partial sequence of SeV HN protein of SEQ ID NO: 12). SEQ ID NO:13 describes a nucleotide sequence encoding the SeV HN protein cytoplasmic domain, encoding SEQ ID NO:14.
SEQ ID NO:18 describes the amino acid sequence of the SeV HN protein ectodomain (partial sequence of SeV HN protein of SEQ ID NO:12). SEQ ID NO:17 describes a nucleotide sequence encoding the SeV HN protein ectodomain, encoding SEQ ID NO:18.

The modified at least one coding sequence of the replication-competent or replication-deficient negative-strand RNA virus may include two or more coding sequences, each coding for a separate polypeptide, for example two, three of four coding sequences. Preferably, the modified at least one coding sequence includes two coding sequences. For example, the modified at least one coding sequence includes the SeV F and HN coding sequences.

In the recombinant replication-competent or replication-deficient RNA virus of the present invention, an endogenous sequence encoding the HN polypeptide may be modified, such that its function or activity is at least partially or completely disrupted. The nucleotide sequence (b) may be inserted in the locus of the HN gene. The endogenous sequence encoding the F polypeptide may be unmodified.

In particular, the sequence encoding the HN polypeptide may be deleted, at least partially or completely. More particular, in the recombinant replication-competent or replication-deficient RNA virus, the RNA molecule does not comprise a sequence encoding a HN polypeptide. Examples of such constructs are described in the Examples (“Type B” constructs). In the recombinant replication-competent or replication-deficient RNA virus of the present invention, an endogenous sequence encoding the HN polypeptide may be deleted, at least partially or completely, and the nucleotide sequence (b) may be inserted in the locus of the HN gene.

In the recombinant replication-competent or replication-deficient RNA virus of the present invention, an endogenous sequence encoding the F polypeptide may be modified, such that its function or activity is at least partially or completely disrupted. The nucleotide sequence (b) may be inserted in the locus of the F gene. The endogenous sequence encoding the HN polypeptide may be unmodified.

In particular, the sequence encoding the F polypeptide may deleted, at least partially or completely. More particular, in the recombinant replication-competent or replication-deficient RNA virus, the RNA molecule does not comprise a sequence encoding a F polypeptide. In the recombinant replication-competent or replication-deficient RNA virus of the present invention, an endogenous sequence encoding the F polypeptide may be deleted, at least partially or completely, and the nucleotide sequence (b) may be inserted in the locus of the F gene.

In the recombinant replication-competent or replication-deficient RNA virus of the present invention, an endogenous sequence encoding the F polypeptide and an endogenous sequence encoding the HN polypeptide may be modified, such that their function or activity is at least partially or completely disrupted. The nucleotide sequence (b) may be inserted in the locus of the F and HN gene.

In particular, the sequence encoding the F and HN polypeptides may by deleted, at least partially or completely. More particular, in the recombinant replication-competent or replication-deficient RNA virus, the RNA molecule does not comprise a sequence encoding an F polypeptide, and said RNA molecule does not comprise a sequence encoding a HN polypeptide. Examples of such constructs are described in the Examples (“Type C” constructs). In the recombinant replication-competent or replication-deficient RNA virus of the present invention, an endogenous sequence encoding the F polypeptide and an endogenous sequence encoding the HN polypeptide may be deleted, at least partially or completely, and the nucleotide sequence (b) may be inserted in the locus of the F and/or HN gene.

In the present invention, the nucleotide sequence (b) may be in operative linkage with the at least one endogenous expression control sequence. The nucleotide sequence (b) may also comprise at least one heterologous expression control sequence in operative linkage with the heterologous coding sequence. The nucleotide sequence (b) may also be in operative linkage with a combination of at least one endogenous viral expression control sequence and at least one heterologous expression control sequence. For example, an endogenous viral promoter sequence may be used. For example, the at least one nucleotide sequence (b), when inserted in the F locus, may be in operative contact with endogenous promotor of the F gene. In another example, the at least one nucleotide sequence (b), when inserted in the HN locus, may be in operative contact with endogenous promotor of the HN gene.

The recombinant replication-competent or replication-deficient negative-strand RNA virus of the invention may further comprise a heterologous sequence encoding a reporter polypeptide (reporter gene), e.g. a fluorescence protein such as GFP, eGFP or a derivative thereof. The reporter gene may be present only for in vitro evaluation of the RNA virus construct described herein. The reporter gene, such as eGFP, may be located downstream the 3′ end of the L gene. The reporter gene has no functional significance to immunization efficacy of the RNA virus constructs. For the medical use of the recombinant replication-competent or replication-deficient negative-strand RNA virus of the present invention, the reporter gene is not required. Rather, the presence of a reporter gene would hinder medical use of the replication-competent or replication-deficient negative-strand RNA virus of the present invention. As used herein, the recombinant replication-competent or replication-deficient negative-strand RNA for medical use, as described herein, preferably does not contain a reporter gene. In particular, as used herein, the recombinant replication-competent or replication-deficient negative-strand RNA for medical use, as described herein, does not contain the GFP gene or the eGFP gene, or a sequence derived therefrom.

In the present invention, the recombinant replication-competent or replication-deficient negative-strand RNA virus of the present invention may be capable of adsorbing at and penetrating into a host cell. The recombinant replication-competent or replication-deficient negative-strand RNA virus of the present invention may also be capable of infecting a eukaryotic cell.

If a sequence encoding an HN protein is present in the recombinant replication-competent or replication-deficient negative-strand RNA virus of the present invention, adsorption to a host cell may mediated by the HN protein. If a sequence encoding an F protein is present in the recombinant replication-competent or replication-deficient negative-strand RNA virus of the present invention, penetration into the cell may initiated by the F protein. If a sequence encoding a SARS-COV-2 S protein is present in the recombinant replication-competent or replication-deficient negative-strand RNA virus of the present invention, adsorbing at and penetrating into a host cell may be mediated by the SARS-CoV-2 S protein.

In the recombinant replication-competent or replication-deficient negative-strand RNA virus of the present invention, the heterologous polypeptide may mediate adsorption at and penetration into a host cell when displayed on the surface of the virus. In particular, the heterologous protein may be the SARS-COV-2 S protein, which can be chimeric protein, as described herein.

In particular, the host cell is a eukaryotic cell. The host cell may be any host cell capable of being infected with a negative-strand RNA virus. The skilled person knows suitable host cells. Suitable host cells are described herein.

The recombinant replication-deficient negative-strand RNA virus of the present invention may be capable of being rescued from eukaryotic cell.

The recombinant replication-deficient negative-strand RNA virus of the present invention may be capable of being propagated in an eukaryotic cell complemented in trans with a sequence deficient in the replication-deficient negative-strand RNA virus, in particular complemented in trans with a sequence encoding the P polypeptide. The cell may also be complemented in trans with a sequence encoding the N and/or L polypeptide. In particular, the recombinant replication-deficient negative-strand RNA virus of the invention is capable of being propagated in a eukaryotic cell complemented in trans with a sequence encoding (i) the P polypeptide, and (ii) the N and L polypeptide.

In the recombinant replication-competent or replication-deficient negative-strand RNA virus of the present invention, the heterologous polypeptide encoded by the at least one nucleotide sequence (b) may comprise a fragment of the Spike polypeptide, in particular the SARS-COV-2 Spike polypeptide.

The SARS-COV-2 S protein can be chimeric protein. In particular, the heterologous polypeptide encoded by the at least one nucleotide sequence (b) may comprise the ectodomain of the SARS-COV-2 Spike polypeptide.

The heterologous polypeptide may be a chimeric SARS-COV-2 Spike polypeptide. In the recombinant replication-competent or replication-deficient negative-strand RNA virus of the present invention, the nucleotide sequence (b) may encode
(i) the ectodomain of the SARS-COV-2 Spike polypeptide, or a fragment thereof,
(ii) the transmembrane domain of the SeV F or HN polypeptide, or a fragment thereof, and
(iii) the cytoplasmic domain of the SeV F or HN polypeptide, or a fragment thereof.

The nucleotide sequence (b) of this construct may comprise a partial sequence of the transmembrane domain of the SARS-COV-2 Spike polypeptide.

In this construct, the nucleotide sequence (b) may be in operative contact with an endogenous SeV expression control sequence, in particular the SeV F or HN expression control sequences, such as the SeV F or HN promoter.

Such constructs are termed herein “Variant 1 constructs”. Examples of Variant 1 constructs are described in the Examples.

In particular, in Variant 1 constructs, the nucleotide sequence (b) may encode
(i) the ectodomain of the SARS-COV-2 Spike polypeptide, or a fragment thereof,
(ii) the transmembrane domain of the SeV F polypeptide, or a fragment thereof, and
(iii) the cytoplasmic domain of the SeV F polypeptide, or a fragment thereof.

The nucleotide sequence (b) of this construct may comprise a partial sequence of the transmembrane domain of the SARS-COV-2 Spike polypeptide.

In particular, the nucleotide sequence (b) may comprise SEQ ID NO:27 or 30. The heterologous polypeptide encoded by nucleotide sequence (b) may comprise SEQ ID NO:31. The ectodomain of the SARS-COV-2 Spike polypeptide may be encoded by SEQ ID NO 21 or 28. The SeV F transmembrane and cytoplasmic domains may be encoded by SEQ ID NO 29. The amino acid sequence of the ectodomain of the SARS-COV-2 Spike polypeptide may comprise SEQ ID NO 22 or 32. The SeV F transmembrane and cytoplasmic domains may comprise SEQ ID NO 33.

In this construct, the nucleotide sequence (b) may be in operative contact with an endogenous SeV expression control sequence, in particular the SeV F expression control sequences, such as an SeV F promoter.

In particular, in Variant 1 constructs, the nucleotide sequence (b) may also encode
(i) the ectodomain of the SARS-COV-2 Spike polypeptide, or a fragment thereof,
(ii) the transmembrane domain of the SeV HN polypeptide, or a fragment thereof, and
(iii) the cytoplasmic domain of the SeV HN polypeptide, or a fragment thereof.

The nucleotide sequence (b) of this construct may comprise a partial sequence of the transmembrane domain of the SARS-COV-2 Spike polypeptide.

In this construct, the nucleotide sequence (b) may be in operative contact with an endogenous SeV expression control sequence, in particular the SeV HN expression control sequences, such as an SeV HN promoter.

As used herein, the term “fragment” includes a partial sequence of a polynucleotide or a polypeptide. The fragment may have essentially the same activity and/or function as the full-length polynucleotide or polypeptide. The fragment may have at least 80%, preferably at least 90%, at least 95%, at least 98% or at least 99% sequence identity with the full-length polynucleotide or polypeptide.

In particular “fragment of a S protein”, or “fragment of a SARS-COV-2 S protein” includes a polypeptide comprising a partial sequence of the full-length S protein. The fragment may have essential the same activity and/or function as the full-length S protein. In particular, a fragment may have at least 80%, preferably at least 90%, at least 95%, at least 98% or at least 99% sequence identity with the full-length S protein. Examples of fragments of a S protein include
(i) the ectodomain and a partial sequence of the transmembrane domain of SARS-COV-2 S protein,
(ii) the ectodomain and the transmembrane domain of SARS-COV-2 S protein,
(iii) the ectodomain, the transmembrane domain, and a partial sequence of the cytoplasmic domain of SARS-COV-2 S protein, or/and
(iv) the ectodomain, the transmembrane domain, and the cytoplasmic domain of SARS-COV-2 S protein, wherein the 19 C terminal amino acid residues of the cytoplasmic domain are deleted.

Preferably, in these fragments of the SARS-COV-2 S polypeptide, the domains are directly adjacent.

The ectodomain of the SARS-COV-2 Spike polypeptide may be encoded by: nucleotide (nt) 1-3.639 of SEQ ID NO: 19, or by SEQ ID NO:21 or 28. The transmembrane domain of the SARS-COV-2 Spike polypeptide may be encoded by: nt 3.640-3.702 of SEQ ID NO: 19, or by SEQ ID NO:23, or by SEQ ID NO:40. The cytoplasmic domain of the SARS-COV-2 Spike polypeptide may be encoded by: nt 3.703-3.819 of SEQ ID NO: 19, or by SEQ ID NO:25, or by SEQ ID NO:42. Positions 2820-2822 of SEQ ID NO:19 describe the STOP codon.

The ectodomain of the SARS-COV-2 Spike polypeptide may comprise: amino acid residue (aa) 1-1.213 of SEQ ID NO:20, or may comprise SEQ ID NO:22. The transmembrane domain of the SARS-CoV-2 Spike polypeptide may comprise: aa 1.214-1.234 of SEQ ID NO:20, or may comprise SEQ ID NO: 24, or may comprise SEQ ID NO:41. The cytoplasmic domain of the SARS-COV-2 Spike polypeptide may comprise: aa 1.235-1.273 of SEQ ID NO:20, or may comprise SEQ ID NO:26, or may comprise SEQ ID NO:43.

The fragment may be in particular an immunogenic fragment, i.e. a polypeptide capable of inducing an immune response in a subject. A particular fragment of a S protein, or fragment of a SARS-CoV-2 S protein may be an immunogenic fragment.

In the recombinant replication-competent or replication-deficient negative-strand RNA virus of the present invention, the nucleotide sequence (b) may encode
(i) the ectodomain of the SARS-COV-2 Spike polypeptide, or a fragment thereof,
(ii) the transmembrane domain of the SARS-COV-2 Spike polypeptide, or a fragment thereof, and
(iii) the cytoplasmic domain of the SeV F or HN polypeptide, or a fragment thereof.

The nucleotide sequence (b) of this construct may comprise a partial sequence of the cytoplasmic domain of the SARS-COV-2 Spike polypeptide.

In this construct, the nucleotide sequence (b) may be in operative contact with an endogenous SeV expression control sequence, in particular an SeV F or HN expression control sequence, such as an SeV F or HN promoter.

Such constructs are termed herein “Variant 2 constructs”. Examples of Variant 2 constructs are described in the Examples.

In particular, in Variant 2 constructs, the nucleotide sequence (b) may encode
(i) the ectodomain of the SARS-COV-2 Spike polypeptide, or a fragment thereof,
(ii) the transmembrane domain of the SARS-COV-2 Spike polypeptide, or a fragment thereof, and
(iii) the cytoplasmic domain of the SeV F polypeptide, or a fragment thereof.

The nucleotide sequence (b) of this construct may comprise a partial sequence of the cytoplasmic domain of the SARS-COV-2 Spike polypeptide. In particular, the nucleotide sequence (b) may comprise SEQ ID NO:36, 44 or 46. The heterologous polypeptide encoded by nucleotide sequence (b) may comprise SEQ ID NO:37, 45 or 47, encoded by SEQ ID N: 36, 44 or 46, respectively. The ectodomain of the SARS-COV-2 may be encoded by SEQ ID NO:21, 28 or 38. The ectodomain of the SARS-COV-2 may comprise SEQ ID NO:22, 32 or 39.

In this construct, the nucleotide sequence (b) may be in operative contact with endogenous SeV expression control sequences, in particular an SeV F expression control sequence, such as an SeV F promoter.

In particular, in Variant 2 constructs, the nucleotide sequence (b) may also encode
(i) the ectodomain of the SARS-COV-2 Spike polypeptide, or a fragment thereof,
(ii) the transmembrane domain of the SARS-COV-2 Spike polypeptide, or a fragment thereof, and
(iii) the cytoplasmic domain of the SeV HN polypeptide, or a fragment thereof.

The nucleotide sequence (b) of this construct may comprise a partial sequence of the cytoplasmic domain of the SARS-COV-2 Spike polypeptide.

In this construct, the nucleotide sequence (b) may be in operative contact with endogenous SeV expression control sequences, in particular an SeV HN expression control sequence, such as an SeV HN promoter.

In the recombinant replication-competent or replication-deficient negative-strand RNA virus of the present invention, the nucleotide sequence (b) may encode
(i) the ectodomain of the SARS-COV-2 Spike polypeptide, or a fragment thereof,
(ii) the transmembrane domain of the SARS-COV-2 Spike polypeptide, or a fragment thereof, and
(iii) the cytoplasmic domain of the SARS-COV-2 Spike polypeptide, or a fragment thereof.

Such constructs are termed herein “Variant 3 constructs”. Examples of Variant 3 constructs are described in the Examples.

In this construct, the nucleotide sequence (b) may be in operative contact with an endogenous SeV expression control sequence, in particular an SeV F or HN expression control sequence, such as an SeV F or HN promoter.

In the present invention, the term “anti-vector antigenicity”, as used herein, describes the capability of the vector to induce an immune response against endogenous components of the vector exposed to the immune system of the host (i.e. the subject to which the vector is administered). Anti-vector antigenicity induces an immune response of the host to the vector, i.e. an anti-vector immunity. In a negative-strand RNA virus, components exposed to the immune system of the host in particular include the surface proteins, such as the F and/or HN polypeptide. At least partial or complete deletion of sequences encoding the surface polypeptide may reduce anti-vector antigenicity

In the present invention, the recombinant replication-competent or replication-deficient negative-strand RNA virus may have a reduced anti-vector antigenicity in comparison to the wildtype form. In the recombinant replication-competent or replication-deficient negative-strand RNA virus of the invention, reduced anti-vector antigenicity may be achieved by at least partial or complete deletion of nucleotide sequences encoding a viral surface polypeptide, in particular F and/or HN, as described herein. For example, the recombinant replication-competent or replication-deficient negative-strand RNA virus particle of the invention may comprise essentially no endogenous (native) surface polypeptide on its surface. The recombinant replication-competent or replication-deficient negative-strand RNA virus particle of the invention may display the at least one heterologous polypeptide on its surface, for example the S protein as described herein. In these constructs, the function and/or activity of the F and/or HN polypeptide may be supplemented by the S protein, in particular the SARS-COV-2 S protein.

In the recombinant replication-competent or replication-deficient negative-strand RNA virus of the present invention, the ectodomain of the SARS-COV-2 Spike polypeptide may comprise an S1 subunit, said S1 subunit comprising a receptor binding domain (RBD), capable of binding to a host cell ACE-2 receptor.

In the recombinant replication-competent or replication-deficient negative-strand RNA virus of the present invention, wherein the S1 subunit can be cleaved off by a host cell ACE-2 protease.

In the recombinant replication-competent or replication-deficient negative-strand RNA virus of the present invention, the ectodomain of the SARS-COV-2 Spike polypeptide may comprise an S2 subunit, capable of fusing the SeV envelope with the host cell membrane or endosome membrane.

In the recombinant replication-competent or replication-deficient negative-strand RNA virus of the present invention, the sequence of the ectodomain of the S protein may comprise at least one sequence modification capable of stabilizing the ectodomain in the prefusion form. For example, the S protein or the ectodomain thereof can be provided in stabilized prefusion form, comprising two consecutive proline substitutions at residues K986 and V987 (K986P and V987P). In the prefusion form, the S protein is unable to mediate the fusion of the viral envelope with the cell/endosome membrane following receptor binding. A sequence encoding the F protein may be present, in order to mediate the fusion of the viral envelope with the cell/endosome membrane.

The recombinant replication-competent or replication-deficient negative-strand RNA virus of the present invention may be capable of inducing a mucosal immune response against the at least one heterologous polypeptide encoded by the at least one nucleotide sequence (b), for example when administered by the intranasal and/or mucosal administration route. The recombinant replication-competent or replication-deficient negative-strand RNA virus of the present invention, in particular the SeV vector of the invention, as described herein, is capable of easily penetrating the mucin layers in the respiratory tract, thus providing ideal conditions to deliver the at least one heterologous polypeptide via the intranasal and/or mucosal administration route, for example in the form of easy-to-handle and easy-to-use intranasal/mucosal spray vaccine.

The mucosal immune response may be an immediate response, as the recombinant replication-competent or replication-deficient negative-strand RNA virus of the present invention can display the at least one heterologous polypeptide on the surface.

The recombinant replication-competent or replication-deficient negative-strand RNA virus of the present invention may be capable of inducing a protective immunity response against a pathogen carrying a polypeptide antigen encoded by the at least one nucleotide sequence (b), in particular against SARS-COV-2. The protective immunity may be a sterilizing immunity. The term “sterilizing immunity”, as used herein, describes an immune response against a pathogen, in particular the SARS-COV-2 virus, that prevents transmission of the pathogen to another, not yet infected subject.

The recombinant replication-competent or replication-deficient negative-strand RNA virus of the present invention can be presented directly to the mucosal immune system, in particular in the course of intra-nasal/mucosal application, just as in natural infection of the respiratory tract by SARS-COV-2. Furthermore, upon entry of mucosal cell of the host, the virus is capable of producing the SARS-COV-2 Spike protein, and displaying it on the cell surface. In this way, an extensive protection, in particular including sterilizing immunity, can be achieved and ensured.

As used herein, the P protein may be encoded by a nucleotide sequence of SEQ ID NO:1, or a sequence having at least 80%, at least 85%, at least 90%, at least 95%, or at least 98% identity thereto.

As used herein, the P protein may comprise an amino acid sequence of SEQ ID NO:2, or a sequence having at least 80%, at least 85%, at least 90%, at least 95%, or at least 98% identity thereto.

As used herein, the deltaP 2-77 protein may be encoded by a nucleotide sequence of SEQ ID NO: 3, or a sequence having at least 80%, at least 85%, at least 90%, at least 95%, or at least 98% identity thereto.

As used herein, the deltaP 2-77 protein may comprise an amino acid sequence of SEQ ID NO:4, or a sequence having at least 80%, at least 85%, at least 90%, at least 95%, or at least 98% identity thereto.

As used herein, the SeV F protein may be encoded by a nucleotide sequence of SEQ ID NO:5, or a sequence having at least 80%, at least 85%, at least 90%, at least 95%, or at least 98% identity thereto.

As used herein, the SeV F protein may comprise an amino acid sequence of SEQ ID NO:6, or a sequence having at least 80%, at least 85%, at least 90%, at least 95%, or at least 98% identity thereto.

As used herein, the SeV F protein transmembrane domain may be encoded by a nucleotide sequence of SEQ ID NO:7, or a sequence having at least 80%, at least 85%, at least 90%, at least 95%, or at least 98% identity thereto.

As used herein, the SeV F protein transmembrane domain may comprise an amino acid sequence of SEQ ID NO:8, or a sequence having at least 80%, at least 85%, at least 90%, at least 95%, or at least 98% identity thereto.

As used herein, the SeV F protein cytoplasmic domain may be encoded by a nucleotide sequence of SEQ ID NO:9, or a sequence having at least 80%, at least 85%, at least 90%, at least 95%, or at least 98% identity thereto.

As used herein, the SeV F protein cytoplasmic domain may comprise an amino acid sequence of SEQ ID NO:10, or a sequence having at least 80%, at least 85%, at least 90%, at least 95%, or at least 98% identity thereto.

As used herein, the SeV HN protein may be encoded by a nucleotide sequence of SEQ ID NO:11, or a sequence having at least 80%, at least 85%, at least 90%, at least 95%, or at least 98% identity thereto.

As used herein, the SeV HN protein may comprise an amino acid sequence of SEQ ID NO:12, or a sequence having at least 80%, at least 85%, at least 90%, at least 95%, or at least 98% identity thereto.

As used herein, the SeV HN cytoplasmic domain may be encoded by a nucleotide sequence of SEQ ID NO:13, or a sequence having at least 80%, at least 85%, at least 90%, at least 95%, or at least 98% identity thereto.

As used herein, the SeV HN cytoplasmic domain may comprise an amino acid sequence of SEQ ID NO: 14, or a sequence having at least 80%, at least 85%, at least 90%, at least 95%, or at least 98% identity thereto.

As used herein, the SeV HN transmembrane domain may be encoded by a nucleotide sequence of SEQ ID NO:15, or a sequence having at least 80%, at least 85%, at least 90%, at least 95%, or at least 98% identity thereto.

As used herein, the SeV HN transmembrane domain may comprise an amino acid sequence of SEQ ID NO:16, or a sequence having at least 80%, at least 85%, at least 90%, at least 95%, or at least 98% identity thereto.

As used herein, the SeV HN ectodomain may be encoded by a nucleotide sequence of SEQ ID NO: 17, or a sequence having at least 80%, at least 85%, at least 90%, at least 95%, or at least 98% identity thereto.

As used herein, the SeV HN ectodomain may comprise an amino acid sequence of SEQ ID NO: 18, or a sequence having at least 80%, at least 85%, at least 90%, at least 95%, or at least 98% identity thereto.

As used herein, the SARS-COV-2 Spike(S) protein may be encoded by a nucleotide sequence of SEQ ID NO:19, or a sequence having at least 80%, at least 85%, at least 90%, at least 95%, or at least 98% identity thereto.

As used herein, the SARS-COV-2 Spike(S) protein may comprise an amino acid sequence of SEQ ID NO:20, or a sequence having at least 80%, at least 85%, at least 90%, at least 95%, or at least 98% identity thereto.

As used herein, the SARS-COV-2 Spike(S) ectodomain may be encoded by a nucleotide sequence of SEQ ID NO:21, 28, or 38 or a sequence having at least 80%, at least 85%, at least 90%, at least 95%, or at least 98% identity thereto.

As used herein, the SARS-COV-2 Spike(S) ectodomain may comprise an amino acid sequence of SEQ ID NO:22, 32, or 39 or a sequence having at least 80%, at least 85%, at least 90%, at least 95%, or at least 98% identity thereto.

As used herein, the SARS-COV-2 Spike(S) transmembrane domain may be encoded by a nucleotide sequence of SEQ ID NO:23 or 40, or a sequence having at least 80%, at least 85%, at least 90%, at least 95%, or at least 98% identity thereto.

As used herein, the SARS-COV-2 Spike(S) transmembrane domain may comprise an amino acid sequence of SEQ ID NO:24 or 41, or a sequence having at least 80%, at least 85%, at least 90%, at least 95%, or at least 98% identity thereto.

As used herein, the SARS-COV-2 Spike(S) cytoplasmic domain may be encoded by a nucleotide sequence of SEQ ID NO:25 or 42, or a sequence having at least 80%, at least 85%, at least 90%, at least 95%, or at least 98% identity thereto.

As used herein, the SARS-COV-2 Spike(S) cytoplasmic domain may comprise an amino acid sequence of SEQ ID NO:26 or 43, or a sequence having at least 80%, at least 85%, at least 90%, at least 95%, or at least 98% identity thereto.

As used herein, a chimeric protein comprising the SARS-COV-2 Spike ectodomain, the SeV F transmembrane domain and the SeV cytoplasmic domain (Variant 1 construct) may be encoded by a nucleotide sequence of SEQ ID NO:27, 30, or 34, or a sequence having at least 80%, at least 85%, at least 90%, at least 95%, or at least 98% identity thereto.

As used herein, a chimeric protein comprising the SARS-COV-2 Spike ectodomain, the SeV F transmembrane domain and the SeV cytoplasmic domain (Variant 1 construct) may comprise an amino acid sequence of SEQ ID NO:31 or 35, or a sequence having at least 80%, at least 85%, at least 90%, at least 95%, or at least 98% identity thereto.

As used herein, a SeV F transmembrane domain and the SeV cytoplasmic domain may be encoded by a nucleotide sequence of SEQ ID NO:29 or a sequence having at least 80%, at least 85%, at least 90%, at least 95%, or at least 98% identity thereto.

As used herein, a SeV F transmembrane domain and the SeV cytoplasmic domain, may comprise an amino acid sequence of SEQ ID NO:33, or a sequence having at least 80%, at least 85%, at least 90%, at least 95%, or at least 98% identity thereto.

As used herein, a chimeric protein comprising the SARS-COV-2 Spike ecto, transmembrane domain and cytoplasmic domain (19 C terminal amino acid residues of the cytoplasmic domain deleted), and the SeV F cytoplasmic domain (Variant 2 construct) may be encoded by a nucleotide sequence of SEQ ID NO:36, 44 or 46, or a sequence having at least 80%, at least 85%, at least 90%, at least 95%, or at least 98% identity thereto.

As used herein, a chimeric protein comprising the SARS-COV-2 Spike ecto, transmembrane domain and cytoplasmic domain (19 C terminal amino acid residues of the cytoplasmic domain deleted), and the SeV F cytoplasmic domain (Variant 2 construct) may comprise an amino acid sequence of SEQ ID NO: 37, 45 or 47, or a sequence having at least 80%, at least 85%, at least 90%, at least 95%, or at least 98% identity thereto.

Another aspect of the present invention relates to a recombinant replication-deficient single-stranded negative-strand RNA virus particle, comprising a nucleocapsid and an envelope, said nucleocapsid comprising in its genome:
(a) a nucleotide sequence encoding the SeV P polypeptide, said nucleotide sequence being modified in comparison to the wildtype form, said modification leading to replication deficiency of the negative-strand RNA virus, and
(b) at least one nucleotide sequence encoding at least one heterologous polypeptide, wherein the at least one heterologous polypeptide is displayed on the surface of the virus particle.

The nucleotide sequence (a) encoding a P polypeptide, said nucleotide sequence being modified in comparison to the wildtype form, and at least one nucleotide sequence (b) encoding at least one heterologous polypeptide are described herein in the context of the replication-deficient negative-strand RNA virus.

Another aspect of the present invention relates to a recombinant replication-competent single-stranded negative-strand RNA virus particle, comprising a nucleocapsid and an envelope, said nucleocapsid comprising in its genome:
(a) a nucleotide sequence encoding the SeV P polypeptide, wherein the P protein exerts in particular its natural function in the viral replication cycle,
(b) at least one nucleotide sequence encoding at least one heterologous polypeptide, wherein the at least one heterologous polypeptide is displayed on the surface of the virus particle.

In particular, the recombinant replication-competent single-stranded negative-strand RNA virus particle is attenuated.

In particular, the virus is a Sendai virus.

In particular, the at least one nucleotide sequence (b) encodes a SARS-COV-2 Spike polypeptide, or a fragment thereof, and said envelope comprises the SARS-COV-2 Spike polypeptide, or fragment thereof being displayed on the surface of the virus particle.

Yet another aspect of the present invention relates to a recombinant replication-deficient Sendai virus, comprising in its genome:
(a) a nucleotide sequence encoding a P polypeptide, said nucleotide sequence being modified in comparison to the wildtype form, said modification leading to replication deficiency of the negative-strand RNA virus, and
(b) at least one nucleotide sequence encoding at least one heterologous polypeptide, wherein the at least one heterologous polypeptide is displayed on the surface of the Sendai virus particle.

The nucleotide sequence (a) encoding a P polypeptide, said nucleotide sequence being modified in comparison to the wildtype form, and the at least one nucleotide sequence (b) encoding at least one heterologous polypeptide are described herein in the context of the replication-deficient negative-strand RNA virus.

In particular, in this aspect, the at least one nucleotide sequence (b) encodes a SARS-COV-2 Spike polypeptide, or a fragment thereof, and said envelope comprises the SARS-COV-2 Spike polypeptide, or fragment thereof being displayed on the surface of the Sendai virus particle.

Yet another aspect of the present invention relates to a recombinant replication-competent Sendai virus, comprising in its genome:
(a) a nucleotide sequence encoding a P polypeptide, wherein the P protein exerts in particular its natural function in the viral replication cycle,
(b) at least one nucleotide sequence encoding at least one heterologous polypeptide, wherein the at least one heterologous polypeptide is displayed on the surface of the virus particle. In particular, the replication-competent Sendai virus particle is attenuated.

In particular, in this aspect, the at least one nucleotide sequence (b) encodes a SARS-COV-2 Spike polypeptide, or a fragment thereof, and said envelope comprises the SARS-COV-2 Spike polypeptide, or fragment thereof being displayed on the surface of the Sendai virus particle.

Yet another aspect of the present invention relates to a recombinant replication-deficient Sendai virus particle, comprising a nucleocapsid and an envelope, said nucleocapsid comprising in its genome:
(a) a nucleotide sequence encoding the SeV P polypeptide, said nucleotide sequence being modified (a) in comparison to the wildtype form, said modification leading to replication deficiency of the Sendai virus, and
(b) at least one nucleotide sequence encoding at least one heterologous polypeptide, wherein the at least one heterologous polypeptide is displayed on the surface of the virus particle.

The nucleotide sequence (a) encoding a P polypeptide, said nucleotide sequence being modified in comparison to the wildtype form, and the at least one nucleotide sequence (b) encoding at least one heterologous polypeptide are described herein in the context of the replication-deficient negative-strand RNA virus.

In particular, in this aspect, the at least one nucleotide sequence (b) encodes a SARS-COV-2 Spike polypeptide, or a fragment thereof, and said envelope comprises the SARS-COV-2 Spike polypeptide, or fragment thereof being displayed on the surface of the Sendai virus particle.

Yet another aspect of the present invention relates to a recombinant replication-competent Sendai virus particle, comprising a nucleocapsid and an envelope, said nucleocapsid comprising in its genome:
(a) a nucleotide sequence encoding the SeV P polypeptide, wherein the P protein exerts in particular its natural function in the viral replication cycle,
(b) at least one nucleotide sequence encoding at least one heterologous polypeptide, wherein the at least one heterologous polypeptide is displayed on the surface of the virus particle.

In particular, the replication-competent Sendai virus is attenuated.

In particular, in this aspect, the at least one nucleotide sequence (b) encodes a SARS-COV-2 Spike polypeptide, or a fragment thereof, and said envelope comprises the SARS-COV-2 Spike polypeptide, or fragment thereof being displayed on the surface of the Sendai virus particle.

Yet another aspect of the present invention relates to a recombinant replication-deficient Sendai virus (SeV) particle, comprising a nucleocapsid and an envelope, said nucleocapsid comprising in its genome:
(a) a nucleotide sequence encoding the SeV P polypeptide, said nucleotide sequence being modified in comparison to the wildtype form, said modification leading to replication deficiency of the Sendai virus, and
(b) at least one nucleotide sequence encoding the SARS-COV-2 Spike polypeptide, or a fragment thereof,
   wherein the SARS-COV-2 Spike polypeptide, or fragment thereof is displayed on the surface of the virus particle.

Yet another aspect of the present invention relates to a recombinant replication-competent Sendai virus (SeV) particle, comprising a nucleocapsid and an envelope, said nucleocapsid comprising in its genome:
(a) a nucleotide sequence encoding the SeV P polypeptide, wherein the P protein exerts in particular its natural function in the viral replication cycle, and
(b) at least one nucleotide sequence encoding the SARS-COV-2 Spike polypeptide, or a fragment thereof,
wherein the SARS-COV-2 Spike polypeptide, or fragment thereof is displayed on the surface of the virus particle.

In particular, the replication-competent Sendai virus is attenuated.

Further aspects of the invention relate to the medical use of the recombinant replication-deficient single-stranded negative-strand RNA virus of the invention. As used herein, the term “virus” includes an RNA molecule, a nucleocapsid, and a virus particle capable of infecting new host cells. In these aspects, it is preferred that the recombinant replication-deficient negative-strand RNA virus of the invention is provided in the form of a virus particle.

An aspect of the present invention relates to a pharmaceutical composition comprising the recombinant replication-deficient or replication-competent negative-strand RNA virus as described herein and/or the recombinant replication-deficient or replication-competent negative-strand RNA virus particle as described herein.

In particular, the pharmaceutical composition comprises the recombinant replication-deficient or replication-competent Sendai virus as described herein, and/or the recombinant replication-deficient or replication-competent Sendai virus particle as described herein.

Yet another aspect of the present invention relates to a vaccine comprising the recombinant replication-deficient or replication-competent negative-strand RNA virus as described herein and/or the recombinant replication-deficient or replication-competent negative-strand RNA virus particle as described herein. The vaccine may be an anti-SARS-COV-2 vaccine or/and a vaccine for prevention of COVID 19.

Yet another aspect of the present invention relates to an immunogenic composition comprising the recombinant replication-deficient or replication-competent negative-strand RNA virus as described herein and/or the recombinant replication-deficient or replication-competent negative-strand RNA virus particle as described herein. The immunogenic composition may elicit an immune response against SARS-COV-2.

Yet another aspect of the present invention relates to the recombinant replication-deficient or replication-competent negative-strand RNA virus as described herein, the recombinant replication-deficient or replication-competent negative-strand RNA virus particle as described herein, the pharmaceutical composition as described herein, the vaccine as described herein, or the immunogenic composition as described herein, for medical use.

In particular, the present invention relates to the recombinant replication-deficient or replication-competent negative-strand RNA virus as described herein, the recombinant replication-deficient or replication-competent negative-strand RNA virus particle as described herein, the pharmaceutical composition as described herein, the vaccine as described herein, or the immunogenic composition as described herein, for use in a method for prevention of a SARS-COV-2 infection.

Yet another aspect of the present invention relates to the recombinant replication-deficient or replication-competent negative-strand RNA virus as described herein, the recombinant replication-deficient or replication-competent negative-strand RNA virus particle as described herein, the pharmaceutical composition as described herein, the vaccine as described herein, or the immunogenic composition as described herein, for use in a method for prevention of COVID 19.

Yet another aspect of the present invention relates to the recombinant replication-deficient or replication-competent negative-strand RNA virus as described herein, the recombinant replication-deficient or replication-competent negative-strand RNA virus particle as described herein, the pharmaceutical composition as described herein, the vaccine as described herein, or the immunogenic composition as described herein, for use as a vaccine. The vaccine may be an anti-SARS-COV-2 vaccine or/and a vaccine for prevention of COVID 19.

The recombinant replication-deficient or replication-competent recombinant replication-deficient negative-strand RNA virus, the recombinant replication-deficient or replication-competent recombinant replication-deficient negative-strand RNA virus particle, the pharmaceutical composition, the vaccine or the immunogenic composition may be administered to a subject in need thereof by the intranasal and/or mucosal route.

Yet another aspect of the present invention relates to an RNA molecule, encoding a recombinant replication-deficient negative-strand RNA virus, as describe herein. The RNA molecule may be an isolated RNA molecule. In particular, the RNA molecule comprises
(a) a nucleotide sequence encoding a P polypeptide, said nucleotide sequence being modified in comparison to the wildtype form, and
(b) at least one nucleotide sequence encoding at least one heterologous polypeptide.

The nucleotide sequence (a) encoding a P polypeptide, said nucleotide sequence being modified in comparison to the wildtype form, and the at least one nucleotide sequence (b) encoding at least one heterologous polypeptide are described herein in the context of the replication-deficient negative-strand RNA virus.

Yet another aspect of the present invention relates to an RNA molecule, encoding a recombinant replication-competent negative-strand RNA virus, as described herein. The RNA molecule may be an isolated RNA molecule. In particular, the RNA molecule comprises
(a) a nucleotide sequence encoding a P polypeptide, wherein the P protein exerts in particular its natural function in the viral replication cycle, and
(b) at least one nucleotide sequence encoding at least one heterologous polypeptide.

In particular, the replication-competent negative-strand RNA virus is attenuated.

In particular, the RNA molecule further comprises
(c) a nucleotide sequence encoding a N polypeptide,
(d) a nucleotide sequence encoding a M polypeptide, and
(e) a nucleotide sequence encoding a L polypeptide.

The nucleotide sequence (c), (d) and (e) are described herein in the context of the replication-competent or replication-deficient negative-strand RNA virus.

Yet another aspect of the present invention is a nucleocapsid, comprising the RNA molecule of the present invention.

The nucleocapsid may consist of single-stranded RNA complexed with nucleoprotein (N). In particular in each case 6 nucleotides of the RNA may be bound by one N protein. The nucleocapsid may also comprise an RNA-dependent RNA polymerase (L) and the cofactor phosphoprotein (P), forming the RNA polymerase complex.

Yet another aspect of the present invention is a DNA molecule, encoding the RNA molecule of the present invention. The DNA molecule may be an isolated DNA molecule. In particular, the DNA molecule may be a cDNA molecule. The cDNA may comprise the genome of the replication-competent or replication-deficient negative-strand RNA virus as described herein. The cDNA may be capable of forming a virus particle in a suitable helper cell. The cDNA may be provided in the form of a plasmid, such as an expression plasmid, capable of being transcribed into an RNA molecule encoding the replication-competent or replication-deficient negative-strand RNA virus as described herein.

Yet another aspect of the invention is a host cell, comprising the replication-competent or replication-deficient negative-strand RNA virus as described herein, the recombinant replication-competent or replication-deficient negative-strand RNA virus particle as described herein, the RNA molecule as described herein, the nucleocapsid as described herein and/or DNA molecule as described herein. In particular, the host cell is a eukaryotic cell. The host cell may be any host cell capable of being infected with a negative-strand RNA virus. The skilled person knows suitable host cells.

Examples include the Vero cell, or a cell derived therefrom, such as the V3-10 helper cell, transfected with (i) a nucleic acid molecule encoding in trans at least the SeV P protein. In particular, the cell may be further transfected with (ii) at least one nucleic acid molecule encoding in trans the native viral proteins N and/or L.

The host cell, in particular the V3-10 helper cell, may be capable of replicating the recombinant replication-deficient negative-strand RNA virus of the present invention. Replication includes propagation of the replication-deficient negative-strand RNA virus of the present invention. An example of such eukaryotic cell capable of replicating the recombinant replication-deficient negative-strand RNA is a cell complemented in trans with a sequence deficient in the replication-deficient negative-strand RNA virus, in particular complemented in trans with a nucleic acid molecule encoding (i) the P polypeptide. The cell may further be transfected with (ii) at least one nucleic acid molecule encoding the N and/or L polypeptide. An Example is the V3-10 helper cell, as described herein.

The host cell, in particular the V3-10 helper cell, may also be capable of rescuing the replication-deficient negative-strand RNA virus of the present invention after being transfected with a nucleic acid molecule encoding the recombinant replication-deficient negative-strand RNA virus of the present invention, for example a DNA molecule as described herein. In particular, the host cell capable of viral rescue is not trans-complemented with exogenous sequences encoding the deficient viral proteins.

The recombinant replication-competent negative-strand RNA virus, as described herein, can be replicated in a host cell, without the need of trans-complementation. An example of such cell is the Vero cell.

The host cell may be a GMP-qualified eukaryotic cell. Suitable GMP-qualified eukaryotic cells are known to the skilled persons. An example is the Vero cell.

The host cell may also be a prokaryotic cell, such as a bacterium, suitable for maintenance and propagation of the DNA molecule as described herein, encoding the replication-competent or replication-deficient negative-strand RNA virus of the present invention. Suitable prokaryotic cells are known to the skilled person.

Yet another aspect of method for prevention of an infection with SARS-COV-2 and/or COVID 19, comprising administering to a subject in need thereof the recombinant replication-competent or replication-deficient negative-strand RNA virus as described herein, the recombinant replication-competent or replication-deficient negative-strand RNA virus particle as described herein, the pharmaceutical composition as described herein, the vaccine as described herein, and/or the immunogenic composition as described herein.

A further aspect of the present invention relates to a method for production of a pharmaceutical composition as described herein, the recombinant replication-competent or replication-deficient recombinant replication-competent or replication-deficient negative-strand RNA virus particle as described herein, the vaccine as described herein, or the immunogenic composition as described herein, comprising formulating the recombinant replication-competent or replication-deficient negative-strand RNA virus as described herein, the RNA molecule as described herein, or/and the nucleocapsid, together with at least one pharmaceutically acceptable excipient.

Yet another aspect of the present invention is a method for production of the recombinant replication-deficient negative-strand RNA virus as described herein,

The method for production of the recombinant replication-deficient negative-strand RNA virus or virus particle as described herein, may comprise
(a) transfecting a eukaryotic host cell with a DNA molecule encoding the RNA molecule of the present invention, said host cell being capable of expressing the SeV polypeptides N, P and L,
(b) cultivating the host cell under conditions such that said DNA molecule is transcribed, and the virus particle is formed, and
(c) isolating the virus particle of (b).

The method for production of the recombinant replication-deficient negative-strand RNA virus as or virus particle described herein, may also comprise
(a) infecting a eukaryotic host cell with a wildtype negative strand RNA virus, and transfecting the eukaryotic host cell with a DNA molecule as described herein, encoding a recombinant replication-deficient RNA virus of the invention, and
(b) cultivating the host cell under conditions such that said DNA molecule is transcribed, and the virus particle is formed, and
(c) isolating the virus particle of (b), wherein the wildtype negative strand RNA virus and the recombinant replication-deficient RNA virus may be derived from the same species.

Yet another aspect of the present invention is a method for production of the recombinant replication-competent negative-strand RNA virus or virus particle as described herein, comprising
(a) transfecting a eukaryotic host cell with a DNA molecule as described herein,
(b) cultivating the host cell under conditions such that said DNA molecule is transcribed, and the virus particle is formed, and
(c) isolating the virus particle of (b).

Yet another aspect of the present invention relates to the use of the host cell, as described herein, for the production of the recombinant replication-competent or replication-deficient negative-strand RNA virus and/or the recombinant replication-competent or replication-deficient negative-strand RNA virus particle, as described herein.

A further aspect of the present invention relates to the use of a recombinant replication-competent or replication-deficient negative-strand RNA virus as described herein, for the manufacture of a medicament for the prevention of an infection with SARS-COV-2 and/or COVID 19.

### Description of the Nucleotide and Amino Acid Sequences

| SEQ ID NO | Description |
|---|---|
| 1 | Nucleotide sequence encoding the Sendai virus (SeV) wildtype P protein |
| 2 | Amino acid sequence of Sendai virus (SeV) wildtype P protein, encoded by SEQ ID |
| | NO: 1 |
| 3 | Nucleotide sequence encoding the SeV P protein with deletion of amino acids 2-77 |
| | (,,deltaP 2-77”) |
| 4 | Amino acid sequence of the SeV P protein with deletion of amino acids 2-77 |
| | (,,deltaP 2-77”), encoded by SEQ ID NO: 3 |
| 5 | Nucleotide sequence encoding the Sendai virus (SeV) SeV F protein (surface protein |
| | for fusion with target cell) |
| 6 | Amino acid sequence of the Sendai virus (SeV) SeV F protein (surface protein for |
| | fusion with target cell), encoded by SEQ ID NO: 5 |
| 7 | Nucleotide sequence encoding the SeV F protein transmembrane domain (partial |
| | sequence of SeV F protein of SEQ ID NO: 5) |
| 8 | Amino acid sequence of the SeV F protein transmembrane domain (partial sequence |
| | of SeV F protein of SEQ ID NO: 6), encoded by SEQ ID NO: 7 |
| 9 | Nucleotide sequence encoding the SeV F protein cytoplasmic domain (partial |
| | sequence of SeV F protein of SEQ ID NO: 5) |
| 10 | Amino acid sequence of the SeV F protein cytoplasmic domain (partial sequence of |
| | SeV F protein of SEQ ID NO: 6), encoded by SEQ ID NO: 9 |
| 11 | Nucleotide sequence encoding the the Sendai virus (SeV) HN protein (surface |
| | protein for binding to the target cell) |
| | Cytoplasmic domain: nt 1-111 |
| | Transmembrane domain: nt 112-174 |
| | Ecto domain: nt 175-1.728 |
| 12 | Amino acid sequence of nucleotide sequence encoding the Sendai virus (SeV) HN |
| | protein (surface protein for binding to the target cell), encoded by SEQ ID NO: 11 |
| 13 | Nucleotide sequence encoding the SeV HN protein cytoplasmic domain (partial |
| | sequence of SeV HN protein of SEQ ID NO: 11) |
| 14 | Amino acid sequence of the SeV HN protein cytoplasmic domain (partial sequence |
| | of SeV F protein of SEQ ID NO: 12), encoded by SEQ ID NO: 13 |
| 15 | Nucleotide sequence encoding the SeV HN protein transmembrane domain (partial |
| | sequence of SeV HN protein of SEQ ID NO: 11) |
| 16 | Amino acid sequence of the SeV HN protein transmembrane domain (partial |
| | sequence of SeV HN protein of SEQ ID NO: 12), encoded by SEQ ID NO: 15 |
| 17 | Nucleotide sequence encoding the SeV HN protein ectodomain (partial sequence of |
| | SeV HN protein of SEQ ID NO: 11) |
| 18 | Amino acid sequence of the SeV HN protein ectodomain (partial sequence of SeV |
| | HN protein of SEQ ID NO: 12), encoded by SEQ ID NO: 17 |
| 19 | Nucleotide sequence encoding the corona virus SARS-COV-2 Spike (S) protein |
| | (surface protein for receptor binding and fusion with the target cell) |
| | Ecto domain: nt 1-3.639 |
| | Transmembrane domain: nt 3.640-3.702 |
| | Cytoplasmic domain: nt 3.703-3.822 |
| 20 | Amino acid sequence of the corona virus SARS-COV-2 Spike (S) protein (surface |
| | protein for receptor binding and fusion with the target cell), encoded by SEQ ID |
| | Nos: 19. |
| | Ectodomain: aa 1-1.213 |
| | transmembrane domain: aa 1.214-1.234 |
| | cytoplasmic domain: aa 1.235-1.273 |
| 21 | Nucleotide sequence encoding the SARS-COV-2 Spike ectodomain (partial sequence |
| | of SEQ ID NO: 19) |
| 22 | Amino acid sequence of the SARS-COV-2 Spike ectodomain (partial sequence of |
| | SEQ ID NO: 20), encoded by SEQ ID NO: 21 |
| 23 | Nucleotide sequence encoding the SARS-COV-2 Spike transmembrane domain |
| | (partial sequence of SEQ ID NO: 19) |
| 24 | Amino acid sequence of the SARS-COV-2 Spike transmembrane domain (partial |
| | sequence of SEQ ID NO: 20), encoded by SEQ ID NO: 23 |
| 25 | Nucleotide sequence encoding the SARS-COV-2 Spike cytoplasmic domain (partial |
| | sequence of SEQ ID NO: 19) |
| 26 | Amino acid sequence of the SARS-COV-2 Spike cytoplasmic domain (partial |
| | sequence of SEQ ID NO: 20), encoded by SEQ ID NO: 25 |
| 27 | Nucleotide sequence encoding a chimeric protein comprising the SARS-COV-2 Spike |
| | ectodomain, the SeV F transmembrane domain and the SeV cytoplasmic domain |
| | (Variant 1 construct). |
| | SARS-COV-2 ecto domain: nt 1-3639 |
| | SeV F transmembrane + cytoplasmic domain: nt 3.640-3.840 |
| 28 | Nucleotide sequence encoding the SARS-COV-2 Spike ectodomain, partial sequence |
| | of SEQ ID NO: 27 |
| 29 | Nucleotide sequence the SeV F transmembrane domain and the SeV cytoplasmic |
| | domain, partial sequence of SEQ ID NO: 27 |
| 30 | Nucleotide sequence encoding a chimeric protein comprising the SARS-COV-2 Spike |
| | ectodomain, the SeV F transmembrane domain and the SeV cytoplasmic domain |
| | (Variant 1 construct), codon-optimized sequence for expression in humans, based on |
| | Wuhan strain + D614G mutation |
| 31 | Amino acid sequence of a chimeric protein comprising the SARS-COV-2 Spike ecto |
| | domain, the SeV F transmembrane domain and the SeV cytoplasmic domain (Variant |
| | 1 construct), encoded by SEQ ID NOs: 27 or 30 |
| | SARS-COV-2 ecto domain: aa 1-1213 |
| | SeV F transmembrane + cytoplasmic domain: aa 1214-1279 |
| 32 | Amino acid sequence of the SARS-COV-2 Spike ectodomain, partial sequence of |
| | SEQ ID NO: 31, encoded by SEQ ID NO: 28 |
| 33 | Amino acid sequence of the SeV F transmembrane domain and the SeV cytoplasmic |
| | domain, partial sequence of SEQ ID NO: 31, encoded by SEQ ID NO: 29 |
| 34 | Nucleic acid sequence encoding a chimeric protein SARS-COV-2 ecto domain |
| | including prefusion stabilizing mutations + SeV F transmembrane + cytoplasmic |
| | domains as codon-optimized version for expression in human cells |
| 35 | Amino acid sequence of a chimeric protein SARS-COV-2 ecto domain including |
| | prefusion stabilizing mutations + SeV F transmembrane + cytoplasmic domains as |
| | codon-optimized version for expression in human cells, encoded by SEQ ID NO: 34 |
| 36 | Nucleotide sequence encoding a chimeric protein comprising the SARS-COV-2 Spike |
| | ecto, transmembrane domain and cytoplasmic domain (19 C terminal amino acid |
| | residues of the cytoplasmic domain deleted), and the SeV F cytoplasmic domain |
| | (Variant 2 construct). Exemplary construct SeV85E-1V2 comprises SEQ ID NO: 36. |
| | Based on Wuhan strain + D614G mutation |
| | SARS-COV-2 ecto domain: nt 1-3.639 |
| | SARS-COV-2 transmembrane domain + cytoplasmic domain without terminal 19 |
| | amino acids (aa): nt 3.640-3.762 |
| | SeV F cytoplasmic domain: nt 3.763-3.891 |
| 37 | Amino acid sequence of a chimeric protein comprising the SARS-COV-2 Spike ecto, |
| | transmembrane domain and cytoplasmic domain (19 C terminal amino acid residues |
| | of the cytoplasmic domain deleted), and the SeV F cytoplasmic domain, encoded by |
| | SEQ ID NO: 36 (Variant 2 construct). |
| 38 | Nucleotide sequence encoding the SARS-COV-2 Spike ecto domain (partial sequence |
| | of SEQ ID NO: 36) |
| 39 | Amino acid sequence of the SARS-COV-2 Spike ecto domain (partial sequence of |
| | SEQ ID NO: 37), encoded by SEQ ID NO: 38 |
| 40 | Nucleotide sequence encoding the SARS-COV-2 Spike transmembrane domain |
| | (partial sequence of SEQ ID NO: 36) |
| 41 | Amino acid sequence of the SARS-COV-2 Spike transmembrane domain (partial |
| | sequence of SEQ ID NO: 37), encoded by SEQ ID NO: 40 |
| 42 | Nucleotide sequence encoding the SARS-COV-2 Spike cytoplasmic domain (partial |
| | sequence of SEQ ID NO: 36) |
| 43 | Amino acid sequence of the SARS-COV-2 Spike cytoplasmic domain (partial |
| | sequence of SEQ ID NO: 37), encoded by SEQ ID NO: 42 |
| 44 | Chimeric protein SARS-COV-2 ecto, transmembrane domain and cytoplasmic |
| | domain without terminal 19 aa + SeV F cytoplasmic domain, codon-optimized |
| | sequences of the sequence above for expression in humans, based on Wuhan sequence |
| | of SARS-COV-2 including the D614G mutation |
| | SeV F cytoplasmic domain: nt 3763-3891 |
| 45 | Amino acid sequence encoded by SEQ ID NO: 44 |
| 46 | Chimeric protein SARS-COV-2 ecto, transmembrane domain and cytoplasmic |
| | domain without terminal 19 aa + SeV F cytoplasmic domain, codon-optimized |
| | sequences of the sequence above for expression in humans, based on the Omicron |
| | variant of SARS-COV-2 |
| | SeV F cytoplasmic domain: nt 3769-3897 |
| 47 | Amino acid sequence encoded by SEQ ID NO: 46 |

The present invention is described in detail and is illustrated by the figures and examples, which are used only for illustration purposes and are not meant to be limiting. Owing to the description and the examples, further embodiments which are likewise included in the invention are accessible to the skilled worker.

### DESCRIPTION OF THE DRAWINGS

FIG. 1: Rescue of recombinant SeV in the RNA Pol II helper system.

FIG. 2: cDNA constructs of SeV-based candidates against SARS-COV-2.

FIG. 3: Immunofluorescence staining of Vero cells transfected with eukaryotic expression plasmids encoding sequences for either SARS-COV-2 SIVI or SARS-COV-2 S2V1 revealed successful expression of both variants.

FIG. 4: The SeV85E-1V2 vaccine vector genome (encoding the SARS-COV-2 S protein as a surface protein instead of SeV proteins F and HN) was successfully rescued in Vero cells.

FIG. 5: The SeV85E-2V1 vaccine vector genome (encoding the SARS-COV-2 S protein as a surface protein instead of SeV proteins F and HN) was successfully rescued in Vero cells.

FIG. 6: The SeV85E-1V2 vaccine vector genome (encoding the SARS-COV-2 S protein as a surface protein instead of SeV proteins F and HN) was successfully rescued in V3-10 helper cells.

FIG. 7: The SeV85E-2V1 vaccine vector genome (encoding the SARS-COV-2 S protein as a surface protein instead of SeV proteins F and HN) was successfully rescued in V3-10 helper cells.

FIG. 8: The SeV85E-1V2 vaccine (encoding the SARS-COV-2 S protein as a surface protein instead of SeV proteins F and HN) was successfully propagated in V3-10 helper cells as reflected by increased fluorescence over time indicating viral spread during the observation period.

FIG. 9: The SeV85E-1V2 vaccine (encoding the SARS-COV-2 S protein as a surface protein instead of SeV proteins F and HN) was successfully propagated in V3-10 helper cells from passage 2 to 4 at defined multiplicities of infection (MOI).

FIG. 10: Immunoblots of SeV85E-1V2 viral particles (purified by ultracentrifugation (UC)) reveal expression of the SARS-COV-2 S protein indicating incorporation of SARS-CoV-2 S proteins directly into SeV85E-1V2 viral particles. Purified viral particles of SeV85E-1V2 pl and SeV85E pl (control virus, not encoding a chimeric SARS-COV-2 protein) were separated by SDS PAGE under reducing (+beta-ME) or non-reducing (−beta-ME) conditions. Expression of virus-encoded proteins SARS-COV-2 S and EGFP was analyzed by immunoblotting. For detection of the SARS-COV-2 S protein, two different antibodies were used (Sigma, GeneTex).

FIG. 11: Dose-dependent IgA levels in nasal washes (A) and brochoalveolar lavages (B) from mice immunized with SeV88-1V2.

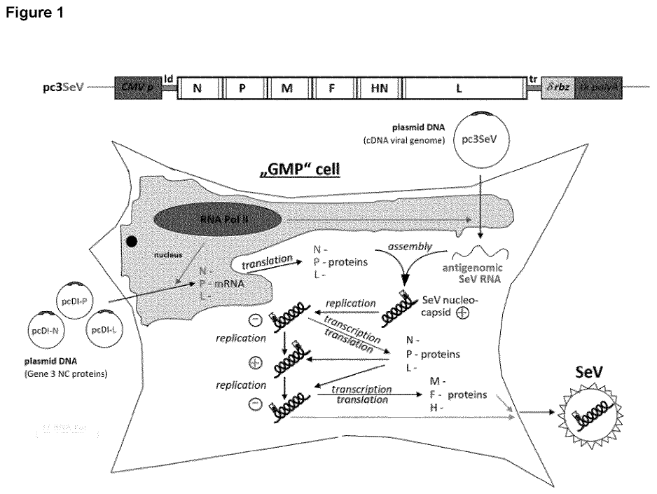

### Example 1

The Examples describes a 2^{nd} generation SARS-COV-2 vaccine. The vaccine comprises a Sendai virus vector, displaying a SARS-COV-2 antigen on the surface.

### Quality and Safety

The candidate vaccines include the quality and safety features of the Sendai virus (SeV) vector, as described in WO 2006/084746:
A key safety feature is partial truncation of a protein of the viral RNA polymerase complex of SeV, achieving a complete replication deficiency. Uncontrolled vector spreading in the vaccinated subject can thus be completely excluded. An infection in a subject treated with the SeV-based vaccine occurs exclusively abortive.
The SeV vector is characterized by genetic stability against recombination events, so that no undesirable (e.g. transgenic) properties can be introduced into the vector.
The genuine ability of the SeV vector to easily penetrate the mucin layers in the respiratory tract provides ideal conditions to deliver a transgenic antigen via the intranasal and/or mucosal administration route in the form of easy-to-handle and easy-to-use intranasal/mucosal spray vaccine.

### Virus Rescue and Propagation

A system supported by cellular RNA polymerase II was established for the primary production of the SeV vectors (the so-called SeV rescue process) in order to be more flexible in the choice of the rescue cells and to be able to use cells already certified for vaccine production. This reduces laborious testing procedures and thus regulatory burden.

WO 2006/084746 discloses SeV rescue and SeV propagation in different cell lines. In the present Examples, the manufacturing system for the SeV vaccine candidates was modified so that the same cells can be used for SeV rescue and subsequent propagation. This substantially simplifies the manufacturing process of the SeV vaccine candidates.

### Candidate SARS-COV-2 Vaccine

In order to achieve an efficient 2^{nd} generation anti-SARS-COV-2 vaccine, the inventors
(i) integrated the SARS-COV-2 S protein in the surface of an SeV-Vaccine vectors, and,
(ii) reduced the amount of SeV proteins F and HN on the surface of the SeV vaccine vectors. With respect to SARS-COV-2 S and SeV F and HN, the vector constructs termed Type A, B and C herein are described in the following Table 1.

TABLE 1
Table 1: Surface protein coding genes in vaccine candidate constructs

| | | Candidate |
|---|---|---|
| | | vaccine(s) |
| | | comprising |
| Type | Description | the construct |
| Type A | -----SARS-COV-2 S---SeV F---SeV HN----- | SeV85E-2V1 |
| Type B | -----SARS-COV-2 S---SeV F----- | |
| Type C | -----SARS-COV-2 S----- | SeV85E-1V1, |
| | | SeV85E-1V2, |
| | | SeV88-1V2 |

If a vaccinated subject has already acquired immunity against SARS-COV-2 in the respiratory tract, for example by SARS-COV-2 infection, the immune system of the subject may recognize the SeV vaccine vector against SARS-COV-2, and the SeV vaccine act as a booster immunization. If a vaccinated subject has not yet acquired immunity against SARS-COV-2 in the respiratory tract, the vaccine vector will induce immunity against SARS-COV-2 via different immune-stimulatory pathways and interactions with the immune system. First, the vaccine vector particles per se are immunogenic for SARS-COV-2 as they display the Spike protein on the surface; in this way they vaccine vector functions as a e.g. nanoparticle vaccine or Virus-like Particle vaccine (VLP). These particles are often recognized by antigen presenting cells of the immune system, e.g. dendritic cells, or are taken up by macrophages, which then cross present parts of the Spike protein to T lymphocytes, which can then differentiate into helper and effector cells. Secondly, the vaccine vector can infect cells, e.g. of the respiratory tract, and express the encoded Spike protein inside of these cells. This will then result in the expression of the Spike protein on the surface of these cells, which can then be recognized by the immune system as infected and will also result in the display of peptides of the Spike protein on MHC-I molecules after standard protein degradation inside of these cells.

To facilitate the detection of the newly generated SeV vaccine particles, the gene for the enhanced green fluorescent protein (eGFP) was inserted into the genome of all SeV vaccine prototypes. The eGFP gene is present only for in vitro evaluation, and has no functional significance to immunization efficacy of the vaccine candidates. After positive evaluation of the vector vaccine candidates, the eGFP gene is removed from the constructs. An example of an RNA virus of the present invention, being free of a sequence encoding GFP, is SeV88-1V2 (Example 5)

### Type A: Expression of the SARS-COV-2 S Protein in Addition to the Expression of the SeV Vector Proteins F and HN (SARS-COV-2 S+SeV F and HN)

In the type A constructs, the SARS-COV-2 S gene was introduced between the SeV P gene (truncated form Pₘᵤₜ) and the M gene.

The type A constructs are designed to determine display of a recombinant viral protein (e.g. the SARS-COV-2 S-Protein) obtained from another family (Coronaviruses) on the envelope of a paramyxovirus (in particular the envelope of SeV), in combination with the proteins F and HN. Such recombinant gene and/or protein may disturb assembly of the virus, and, if the protein is displayed on the surface, may interact with F and/or HN, so that that adsorption and penetration capacity may be negatively impacted.

Adsorption and penetration are essential steps in the infection of a host cell, in both the vaccine production of a replication deficient viral vector, as well as in eliciting the immune response in a subject.

The SARS-COV-2 S gene has a length of more than 3 kb. It had to be determined if a recombinant S gene is genetically stable in the SeV vector and thus is permanently present in the SeV vaccine vector genome. With regard to production of a vector vaccine, this aspect can in no way be considered as trivial. In the type A viral vectors, the SARS-COV-2 gene/protein is not essential for propagation and/or infection. There is thus a considerable risk that such non-essential gene is lost or inactivated during virus propagation if it has any disadvantages in view of the wild-type, leading to a reduced propagation capacity.

In the type A constructs, the SARS COV-2 S protein may be provided in stabilized prefusion form, for example by two consecutive proline substitutions at residues K986 and V987 (K986P and V987P).

### Type B: Expression of the SARS-COV-2 S Protein in Addition to the Expression of the SeV-Vector Proteins F (SARS-COV-2 S+SeV-F)

In type B constructs, the SeV HN gene was deleted. The SARS-COV-2 S gene was introduced between the SeV F gene and the L gene, i.e. replaces the HN gene.

Up to now no studies are available demonstrating an “interfamiliar” substitution of such functionally essential proteins between the completely different virus families of paramyxoviruses and coronaviruses, without a loss of function. Importantly, the function of surface proteins of paramyxoviruses and coronaviruses is not equivalent. Paramyxoviruses display two proteins on the surface: adsorption to a host cell is mediated by the HN protein and penetration (fusion) into the cell is initiated by the F protein. In contrast, in coronaviruses, in particular in SARS-COV-2, adsorption and penetration are mediated by a single surface protein, the S protein. The inventors demonstrated that a SeV virus, in which the F and HN proteins are partially or completely replaced by the SARS-COV-2 S protein, could form recombinant SeV virus particles capable of adsorption at the target cell, and penetration into the cell, and are suitable as anti-SARS-COV-2 vaccine.

Furthermore, in the B type candidate vaccines (SARS-COV-2 S+SeV-F), the amount of SeV surface proteins was reduced, as SeV-HN is no longer present in the SeV vaccine vector genome leading to a reduction in anti-SeV vector immunity.

The penetration mechanism differs in paramyxoviruses (fusion with the host cell membrane) and coronaviruses (mostly endocytosis). Penetration by membrane fusion in coronaviruses has been described, but seems to be less efficient than endocytosis (Jackson, C. B., Farzan, M., Chen, B. et al. Mechanisms of SARS-COV-2 entry into cells. Nat Rev Mol Cell Biol 23, 3-20 (2022). https://doi.org/10.1038/s41580-021-00418-x). In this regard, an “interfamiliar” substitution, as described above, may have a negative impact upon the penetration mechanism and thus replication efficacy.

Adsorption of SARS-COV-2 starts with docking of the virus at the ACE-2 receptor at the host (target) cell in a “prefusion form”. In the prefusion form, the receptor binding domain (RBD) is located inside the S protein due to its natural folding properties. However, by hinge-like movements, the RBD domain repeatedly moves to the surface of the S-protein and can thereby dock to the target cell ACE-2 receptor. Binding to the ACE-2 receptor triggers a conformational rearrangement of the S protein from the metastable prefusion conformation to a highly stable postfusion conformation, which facilitates/stabilizes subsequent membrane fusion. However, the ACE-2 protein acts not only as receptor for the S protein, but also as extracellular peptidase that cuts between the S1 and S2 domains of the SARS-COV-2 S protein. S1 is thereby cleaved off, while S2 fuses with the host cell membrane. The S1 subunit contains the RBD, which binds to the target cell (host cell) receptor. The S2 subunit mediates the fusion of the viral envelope with the cell/endosome membrane following receptor binding. The random conformational change of the RBD is influenced by the ACE-2 receptor: the ACE-2 receptor binds the RBD and stabilizes the postfusion form over the prefusion form.

For many viral fusion glycoproteins, prefusion stabilization by sequence modification can be induced by specific exchange of amino acids at the cleavage site, as described herein, and generally prevents a more conformationally stable postfusion structure, which, however, is significantly more susceptible to heat stress and to freeze-thaw cycles, as well as exhibiting a significant loss of efficacy following storage at room temperature. Accordingly, a prefusion stabilization usually results in significantly increased yields in the recombinant expression of numerous viral fusion glycoproteins. Moreover, prefusion-stabilized viral glycoproteins are usually better/stronger immunogens than their non-stabilized wild-type counterparts, as neutralization by antibodies rather addresses the prefusion than the postfusion conformation.

In the type B constructs, the SARS COV-2 S protein may be provided in the stabilized prefusion form, for example by two consecutive proline substitutions at residues K986 and V987 (K986P and V987P).

The type B candidate vaccines (expression of a prefusion-stabilized SARS-COV-2 S protein together with the SeV vector protein F, but not SeV HN(=SARS-COV-2 S+SeV F), are designed for more efficient vector production and an enhanced immunogenicity against the SARS-COV-2 S protein by stabilization in the prefusion conformation, and at the same time the proportion of SeV vector proteins in the SeV vaccine particles is reduced by omitting the SeV vector protein HN, which should significantly reduce the anti-vector vaccine immunogenicity and thus enable significantly more efficient re-vaccinations (e.g., booster vaccinations).

In the type B SeV constructs the SARS-COV-2 S protein performs receptor binding instead of the SeV HN protein. The S protein represents an essential component of the SeV vaccine vector. The SARS-CoV-2 S gene is an essential and thus mandatory gene of the SeV vaccine vector, the loss or damage of which would completely disable the replication capacity of the recombinant SeV vaccine virus in a transcomplemented cell.

In the type B constructs, no HN coding sequence may be present. In type B constructs, adsorption of the SeV vector particle at the ACE-2 receptor of the target cell may be mediated by the prefusion-stabilized SARS-COV-2 S protein. In type B constructs, SeV F protein could mediate the fusion of the viral envelope with the cell or endosomal membrane. It was, however, unknown to the inventors if the prefusion-stabilized S protein and the SeV F protein could functionally interact so that those type B constructs, containing a prefusion-stabilized SARS-COV-2 S protein, could adsorb to and fuse with the host cell.

### Type C: Expression of SARS-COV-2 S Protein Only (SARS-COV-2 S “Only”); No Expression of SeV Vector Proteins F and HN

In type C constructs, the SeV HN and F genes were deleted. The SARS-COV-2 S gene was introduced between the SeV M and L genes, i.e. replaces the F and HN genes.

This approach (SARS-COV-2 S “only”) appeared to be the most interesting of approaches A, B and C, since all surface proteins of the SeV vector are eliminated and thus cannot function as antigens for anti-vector immunity.

Due to replication deficiency, the vaccine vector will not propagate in the subject. Importantly, any immunity the subject has developed against a vaccine vector (as is the case, for example, with an adenovirus vector) would not interfere with the development of immunity against SARS-COV-2, as the type C vaccine vector do not display vector proteins on the surface.

In the type C vaccine vectors, the SARS-COV-2 S protein is responsible for adsorption to and penetration into the target cell, i.e. is an essential protein. The function of the S protein is required for replication/propagation of the vaccine vectors, i.e. the vaccine vectors can only replicate if the S gene is intact, and the intact S gene is selected during propagation.

By deletion of the SeV genes F and HN, chimeric SeV vectors of type C have approximately the same genome length as SeV wildtype viruses. The adsorption and penetration capability of the SARS-CoV-2 protein is comparable to that of the SeV F and HN proteins, and they replicate with similar efficiency to SeV wildtypes.

Preferably, a prefusion stabilization sequence is not present in Type C constructs. In the Type C constructs, the SARS COV-2 S protein may be provided in a form capable of conformational change between the prefusion form and the postfusion form, and capable of being cleaved by the ACE-2 receptor protease of the host cell.

### Example 2

Variants of SARS-COV-2 S Protein: Chimeric Constructs, Combining the S Protein Ectodomain with Cytoplasmic and/or Transmembrane Domain of SeV F and/or HN

In parallel to the considerations on the combinatory of the surface proteins (i.e. arrangement, distance and number of surface protein molecules on a virus particle), it had to be investigated whether and in which constellations the SARS-COV-2 S protein is incorporated into the SeV vector envelope. If the SeV surface proteins are involved in concentrating the surface proteins in the cytoplasmic membrane of the host cell in addition to the SeV matrix protein (M), incorporation of SARS-COV-2 S protein into the vaccine vector envelope may have a negative impact on the budding process.

It has to be tested if portions of the SeV vector surface proteins F and HN are required, for example in virus assembly, for the interaction with the SeV vector protein M (Matrix protein). The three different design types (A-C) of the vaccine candidates were each designed with three different S proteins, including: (i) the ectodomain ecto from the SARS-COV-2 S protein; (ii) the transmembrane domain tm and/or the (iii) cytoplasmic domain ct derived from either SARS-COV-2 or SeV (Table 2). This results in the following three variants per construction type A-C:

TABLE 2
Table 2: Variants of SARS-COV-2 S proteins

| | | | Candidate |
|---|---|---|---|
| | | | vaccine(s) |
| | | | comprising |
| Variant | Domains | Description | the construct |
| Variant V1 | SARS-COV-2 ecto, | SeV/SARS-COV-2 | SeV85E-2V1, |
| | SeV tm and SeV ct | chimeric protein | SeV85E-1V1 |
| Variant V2 | SARS-COV-2 ecto, | SeV/SARS-COV-2 | SeV85E-1V2 |
| | SARS-CoV-2 tm | chimeric protein | |
| | and SeV ct | | |
| Variant V3 | SARS-COV-2 ecto, | | |
| | SARS-CoV-2 tm | | |
| | and SARS-COV-2 ct | | |

Exemplary construct SeV85E-1V2 comprises SEQ ID NO:36. The chimeric polypeptide comprising the SARS-COV-2 Spike ecto, transmembrane domain and cytoplasmic domain (19 C terminal amino acid residues deleted), and the SeV F cytoplasmic domain is encoded by SEQ ID NO:36, and has the amino acid sequence of SEQ ID NO:37.

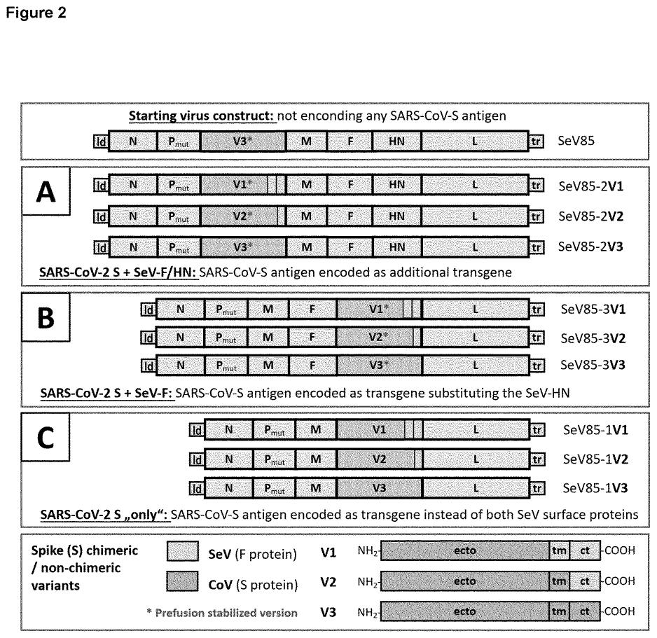

In total 9 different SeV vaccine constructs were designed (FIG. 2).

### Example 3

From the multitude of possible combinations, a meaningful and promising selection (prioritization) of SeV vaccine constructs had to be made, which is compiled in FIG. 2 in the form of genome maps. SeV85E-2V1 (Type A), SeV85E-1V1 (type C) and SeV85E-1V2 (Type C) were evaluated in six steps outlined in Table 3:

TABLE 3
Table 3: Evaluation of SeV vaccine constructs

| Step | |
|---|---|
| 1 | Plasmid-based expression of the chimeric SARS-COV-2 S proteins of the prioritized |
| | SeV vaccine candidates in cell culture (control experiment for the verification of the |
| | structurally correct expression of the chimeric SARS-COV-2 S proteins) |
| 2 | Rescue, i.e. initial production of the SeV vaccine candidates in GMP qualified Vero |
| | cells and V3-10 helper cells |
| 3 | Propagation of the SeV vaccine candidates in in helper cells transfected with a nucleic |
| | acid molecule encoding in trans at least the SeV P protein, to provide sufficient |
| | amounts of virus for the subsequent in vivo tests (animal models, clinical trials) |
| 4 | Evidence for integration of SARS-COV-2 S protein in SeV vaccine candidate virus |
| | particles |
| 5 | Stability of the expression of the SARS-COV-2 S protein by the SeV vaccine |
| | candidates in cell culture |
| 6 | Characterization of the immunological response after infection of murine test animals |
| | with the SeV vaccine candidates |

### Step 1

### Plasmid-Based Expression of the Chimeric SARS-COV-2 S Proteins of the Prioritized SeV Vaccine Candidates in Cell Culture (Control Experiment for the Verification of the Structurally Correct Expression of the Chimeric SARS-COV-2 S Proteins)

Plasmid-based expression of the chimeric SARS-COV-2 S proteins of the SeV vaccine candidates SeV85E-2V1, SeV85E-1V1 and SeV85E-1V2 in a cell culture has verified the expression of the correct structure of the chimeric SARS-COV-2 S proteins.

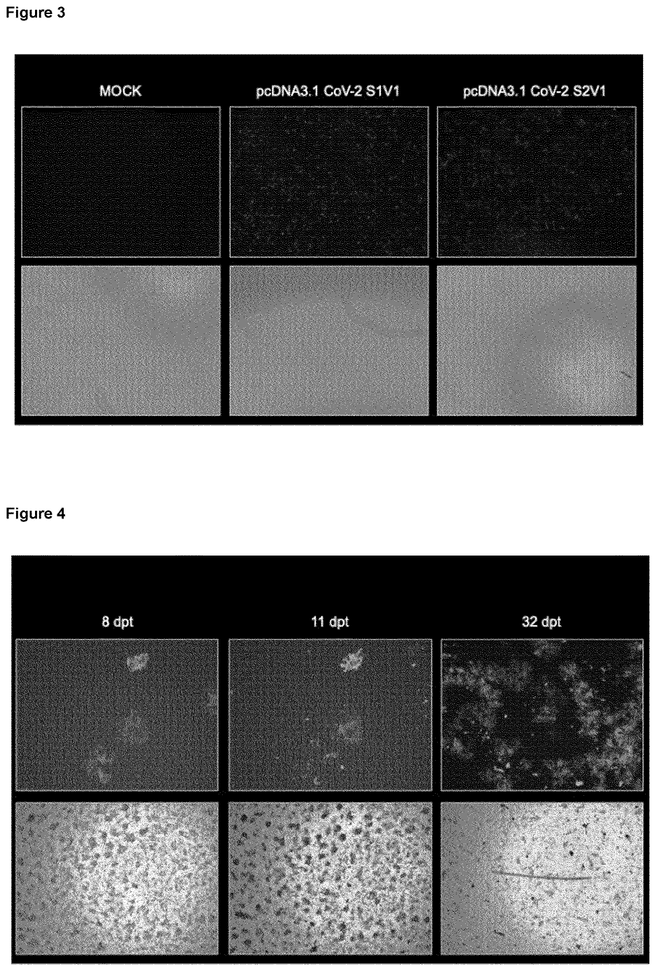

Vero cells were transfected with eukaryotic expression plasmids pcDNA3.1 CoV-2 S2V1 (Type A), pcDNA3.1 CoV-2 SIVI (Type C), and pcDNA3.1 CoV-2 S1V2 (Type C), with the coding sequences for SARS-COV-2 S2V1, SARS-COV-2 S1V1 and SARS-COV-2 S1V2, respectively, or were not transfected (MOCK=negative control). 48 hours post-transfection, cells were stained with a primary antibody directed against SARS-COV-1/2 S protein. A goat anti-mouse Alexa Fluor 546 was used as the secondary antibody. Structural integrity of the coding chimeric sequences for either SARS-COV-2 S2V1, SARS-COV-2 S1V1 or SARS-COV-2 S1V2 is shown by red signals (FIG. 3, middle and right panel).

### Step 2

Rescue, i.e. Initial Production of the SeV Vaccine Candidates in GMP Qualified Vero Cells and V3-10 Helper Cells

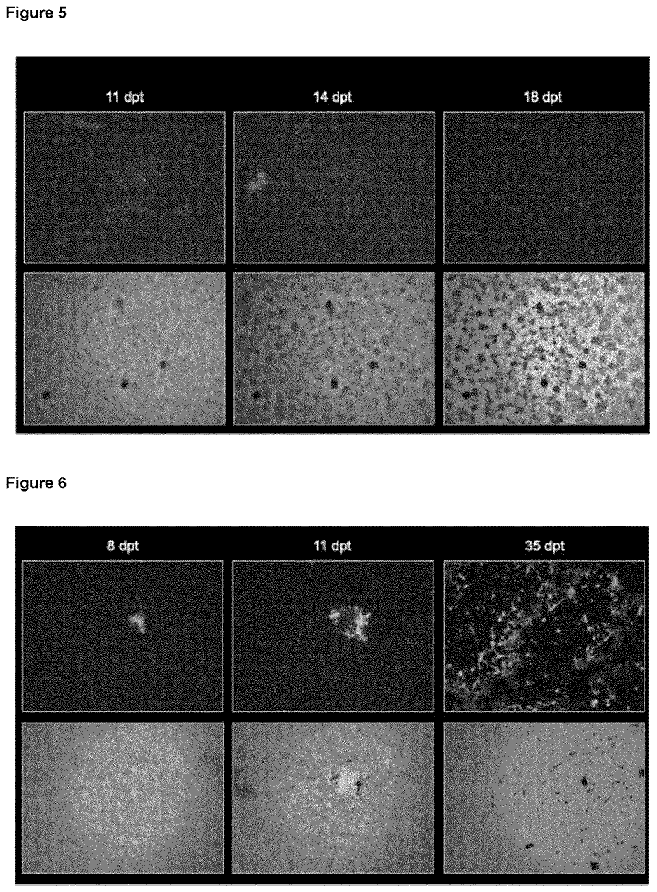

Rescue, i.e. the initial production of recombinant virus particles, was successful with both the SeV vaccine candidate SeV85E-1V2 (type C) and the SeV vaccine candidate SeV85E-2V1 (Type A) in GMP qualified Vero cells (FIGS. 4 and 5).

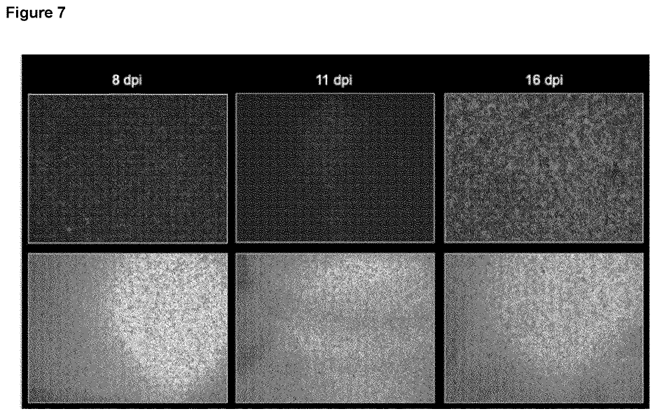

In addition, rescue was achieved with the SeV vaccine candidates SeV85E-1V2 (type C) and SeV85E-2V1 (type A) in the helper cell line V3-10 (FIGS. 6 and 7).

V3-10 cells are Vero-based helper cells stably transfected with plasmids encoding at least SeV proteins N and P. This helper cell line can be used for rescue and propagation of replication-deficient SeV vectors (here: Pₘᵤₜ). Pₘᵤₜ describes an N-terminally truncated form of the P protein.

### Step 3

### Propagation of the SeV Vaccine Candidates in GMP-Qualified Vero Cells and in Helper Cells to Provide Sufficient Amounts of Virus for the Subsequent In Vivo Tests (Animal Models, Clinical Trials)

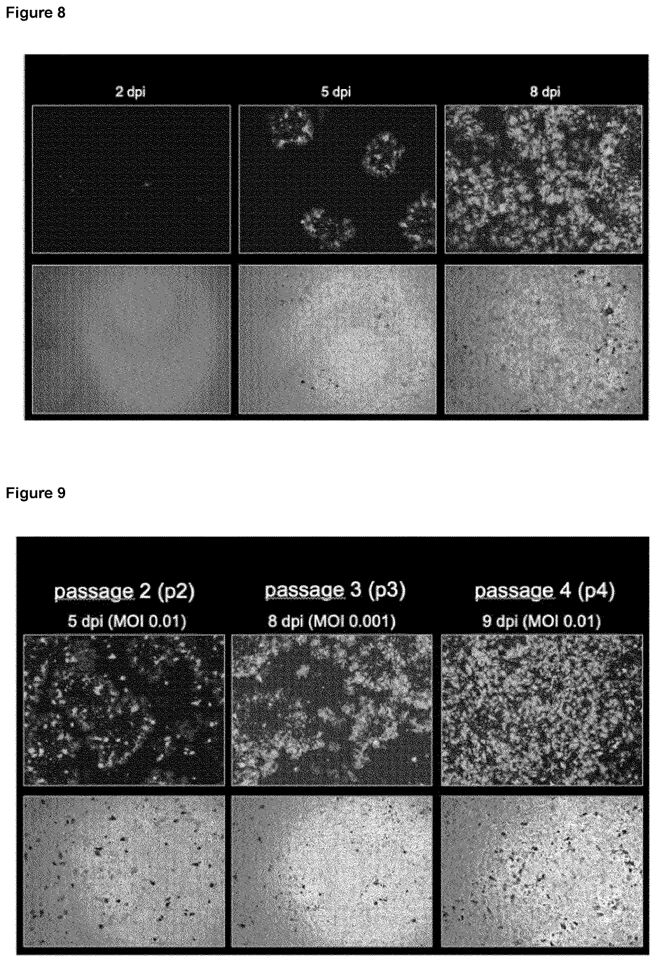

Propagation in V3-10 helper cells was successful for the SeV vaccine candidate SeV85E-1V2 (type C). With this type C prototype, the titers required for animal testing could already be achieved (FIGS. 8 and 9).

### Step 4

### Evidence for Integration of SARS-COV-2 S Protein in SeV Vaccine Candidate Virus Particles

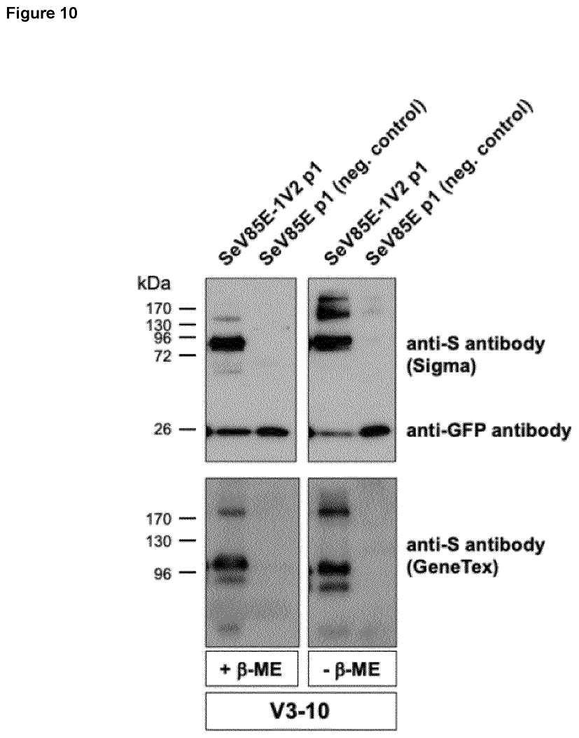

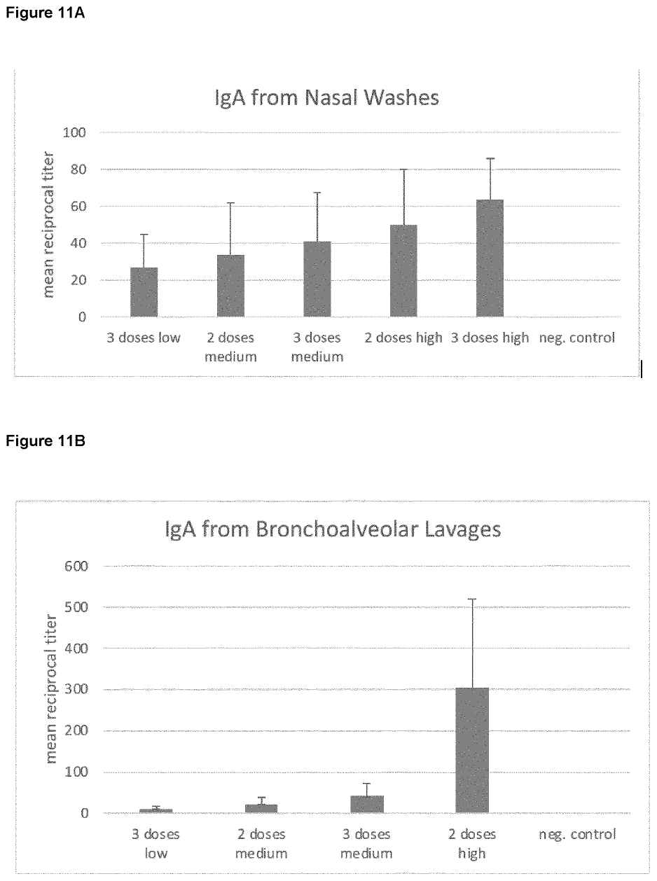

Immunoblots of SeV85E-1V2 (type C) virus particles (purified by ultracentrifugation) showed expression of SARS-COV-2 S protein, demonstrating incorporation of SARS-COV-2 S proteins directly into the SeV85E-1V2 (type C) virus particle (FIG. 10).

### Step 5

### Stability of the Expression of the SARS-COV-2 S Protein by the SeV Vaccine Candidates in Cell Culture.

The stability of SARS-COV-2 S protein expression is demonstrated by immunoblot analysis in supernatants and whole cell lysates collected from the various viral passages.

### Step 6

Characterization of the Immunological Response after Infection of Murine Test Animals with the SeV Vaccine Candidates

The humoral and cellular immune response of mice vaccinated with the SeV vector constructs is determined.

### Conclusion

The Example demonstrates that recombinant SeV candidate vaccines displayed the chimeric SARS-COV-2 S protein on the surface and, at the same time, have substantially reduced expression of SeV surface antigens. SeV vaccine candidates could be rescued from GMP-qualified Vero cells. The SeV candidate vaccine could be propagated in Vero cell-derived helper cells (e.g. cell line V3-10), being transfected with a nucleic acid molecule encoding in trans at least the SeV P protein. Cell culture in Vero cell-derived helper cells can provide sufficient amounts of virus quantities for in vivo tests (animal models & clinical trials).

### Example 4

### Attenuation of Replication-Competent SeV

Here, we identified differences in attenuation between two viral vector constructs of our SARS-CoV-2 vaccines that both contain the attenuating mutation of the exchange of both Sendai surface proteins F and HN entirely against the SARS-COV-2 surface protein S. One vaccine candidate is replication competent and still contains the unmodified Sendai P gene (1); the other vaccine candidate (2) contains the additional attenuating deletion of amino acids 2 to 77 within the P protein, which renders it replication-deficient. Compared to the unmodified recombinant Sendai virus vector, a huge decrease in replication efficacy can be observed increasing with additional attenuating mutations. The following viral titers were obtained from viral replication studies under cell culture conditions in Vero cells (replication-competent SeV) and V3-10 helper cells (replication-deficient SeV).

| | unmodified | (1) replication | (2) replication- |
|---|---|---|---|
| | replication | competent | deficient |
| | competent | Se V with | SeV with |
| | SeV with | SARS- | SARS- |
| | Sendai F | COV-2 S | COV-2 S |
| | and HN as | protein as only | protein as only |
| | surface proteins | surface protein | surface protein |
| Cell infectious unit/mL | 5x 10e8 | 2x 10e6 | 2x 10e4 |
| (ciu/mL) | | | |
| Attenuation factor | 100% | 0.4% | 0.004% |

In conclusion, a replication-competent SeV (1), wherein the surface proteins F and HN are replaced by SARS-COV-2 surface protein S, is attenuated in view of an unmodified replication-competent SeV, making such recombinant virus suitable as an immunogenic agent or vaccine against SARS-COV-2.

Furthermore, such modified replication-competent SeV (1) has an increased production efficacy, compared with replication-deficient SeV (2).

### Example 5

### Immunogenic Effect of SeV88-1V2

BAL samples represent mucosal tissue samples of the lower respiratory tract, NW samples of the upper respiratory tract. Both samples can help to describe the immunological status of these tissues as they contain constituents of the immune system, such as macrophages, lymphocytes, neutrophils or antibodies released from surrounding cells of the tissue or immune cells. Specific antibodies against a pathogen can contribute to a protective immune response by binding and neutralising invading pathogens such as viruses.

### Methods

Female BALB/c mice (4 weeks of age) in groups of 5 mice were intranasally (IN) immunized two or three times every three weeks, with escalating vaccine doses. For collection of bronchoalveolar lavages (BAL) and nasal washes (NW) mice were sacrificed two weeks after the last immunization by cervical dislocation under anaesthetization.

IgA antibodies against the SARS-COV-2 Spike protein were measured by an enzyme-linked immunosorbent assay (ELISA) in a microtiter format. Serial two-fold dilutions were prepared and incubated in an antigen-precoated microtiter plate. Subsequently, a horseradish-peroxidace labelled anti-mouse IgA was added. After 1 h the substrate 3,3′,5,5′ Tetramethylbenzidine (TMB) was added and the reaction was stoppe after 30 min with NaH₂SO₄. Subsequently, the colorimetric conversion was measured in a spectrophotometer at 450 nm. The titer is expressed as reciprocal values of the mean.

### Nasal Washes (NW)

| | DOSE | SeV88-1V2 | MEAN | SD |
|---|---|---|---|---|
| G1 | 1x 10e4 ciu* | 3 doses low | 27 | 18 |
| G2 | 1x 10e5 | 2 doses medium | 34 | 28 |
| G3 | 1x 10e5 | 3 doses medium | 41 | 27 |
| G4 | 1x 10e6 | 2 doses high | 50 | 30 |
| G5 | 1x 10e6 | 3 doses high | 64 | 22 |
| G6 | PBS | neg. control | 0 | 0 |

### Bronchoalveloar Lavages (BAL)

| | DOSE | SeV88-1V2 | MEAN | SD |
|---|---|---|---|---|
| G1 | 1x 10e4 ciu* | 3 doses low | 10 | 6 |
| G2 | 1x 10e5 | 2 doses medium | 21 | 17 |
| G3 | 1x 10e5 | 3 doses medium | 41 | 30 |
| G4 | 1x 10e6 | 2 doses high | 304 | 215 |
| G5 | PBS | neg. control | 0 | 0 |

*cell infectious units

### Results

The search of specific IgA in IN-immunized mice showed that the immunization was able to stimulate a specific mucosal immune response against SARS-COV-2. Immunization with SeV88-1V2 dose-dependently induced specific IgA antibodies. On the basis of the specific IgA levels, the concentration of IgA was higher in BAL than in NW. In particular, groups G4 and G5 revealed the highest titers in comparison to groups immunized with a lower dose or fewer doses of SeV88-1V2 (FIG. 11).

The invention also encompasses the following items:
1. A recombinant replication-deficient negative-strand RNA virus, comprising in its genome:
   (a) a nucleotide sequence encoding a P polypeptide, said nucleotide sequence being modified in comparison to the wildtype form, said modification leading to replication deficiency of the negative-strand RNA virus, and
   (b) at least one nucleotide sequence encoding at least one heterologous polypeptide, wherein the at least one heterologous polypeptide is displayed on the surface of the virus particle.
2. The recombinant replication-deficient negative-strand RNA virus according to item 1, which is a Paramyxovirus, Artovirus, Bornavirus, Filovirus, Lispivirus, Mymonavirus, Nyamivirus, Pneumovirus, Rhabdovirus, Sunvirus, or Xinmovirus.
3. The recombinant replication-deficient negative-strand RNA virus according to item 1 or 2, which is a Sendai virus.
4. The recombinant replication-deficient negative-strand RNA virus according to any one of the preceding items, wherein the modification in the nucleotide sequence (a) is located in the nucleotide sequence encoding the N terminus of the P polypeptide.
5. The recombinant replication-deficient negative-strand RNA virus according to any one of the preceding items, wherein the modification in the nucleotide sequence (a) is a deletion in the nucleotide sequence encoding the N terminus of the P polypeptide.
6. The recombinant replication-deficient negative-strand RNA virus according to any one of the preceding items, wherein the modification in the nucleotide sequence (a) is
   (i) a deletion of the nucleotide sequence encoding the amino acids 2-77 of the P polypeptide, in comparison to the wildtype form, or
   (ii) a partial sequence of (i), leading to replication deficiency of the RNA virus.
7. The recombinant replication-deficient negative-strand RNA virus according to any one of the preceding items, which is transcription-competent.
8. The recombinant replication-deficient negative-strand RNA virus according to any one of the preceding items, wherein the genome further comprises
   (c) a nucleotide sequence encoding a negative-strand RNA virus N polypeptide,
   (d) a nucleotide sequence encoding a negative-strand RNA virus M polypeptide, and
   (e) a nucleotide sequence encoding a negative-strand RNA virus L polypeptide.
9. The recombinant replication-deficient negative-strand RNA virus according to any one of the preceding items, wherein the heterologous polypeptide comprises a heterologous antigen.
10. The recombinant replication-deficient negative-strand RNA virus according to any one of the preceding items, wherein the heterologous polypeptide comprises a viral antigen.
11. The recombinant replication-deficient negative-strand RNA virus according to item 10, wherein the viral antigen and the negative-stranded RNA virus are selected from viruses of different families.
12. The recombinant replication-deficient negative-strand RNA virus according to any one of the preceding items, wherein the heterologous polypeptide comprises an antigen from a virus which is not a negative-strand RNA virus.
13. The recombinant replication-deficient negative-strand RNA virus according to any one of the preceding items, wherein the heterologous polypeptide comprises an antigen from a positive-strand virus, such as a coronavirus.
14. The recombinant replication-deficient negative-strand RNA virus according to any one of the preceding items, wherein the negative-strand RNA virus is a paramyxovirus, such as a Sendai virus, and the heterologous polypeptide comprises an antigen from a coronavirus.
15. The recombinant replication-deficient negative-strand RNA virus according to any one of the preceding items, wherein the heterologous polypeptide comprises a coronavirus Spike protein (S protein), or an immunogenic fragment thereof.
16. The recombinant replication-deficient negative-strand RNA virus according to any one of the preceding items, wherein the coronavirus is SARS-COV-2.
17. The recombinant replication-deficient negative-strand RNA virus according to any one of the preceding items, wherein the at least one nucleotide sequence (b) is located in the RNA molecule between two coding sequences, at a position upstream the 5′ terminal coding sequence and/or downstream the 3′ terminal coding sequence.
18. The recombinant replication-deficient negative-strand RNA virus according to any one of the preceding items, wherein the genome further comprises
   (f) a nucleotide sequence encoding a negative-strand RNA virus F polypeptide, and
   (g) a nucleotide sequence encoding a negative-strand RNA virus HN polypeptide.
19. The recombinant replication-deficient negative-strand RNA virus according to any one of the preceding items, wherein the at least one nucleotide sequence (b) is inserted in or replaces at least partially at least one native (endogenous) sequence encoding at least one viral polypeptide, such that the function or activity of the at least one endogenous viral polypeptide is disrupted at least partially or completely.
20. The recombinant replication-deficient negative-strand RNA virus according to any one of the preceding items, wherein the nucleotide sequence (b) encoding the at least one heterologous polypeptide is essential to viral replication and/or propagation.
21. The recombinant replication-deficient negative-strand RNA virus according to any one of the preceding items, wherein the heterologous polypeptide encoded by sequence (b) is functionally expressed (in biologically active form).
22. The recombinant replication-deficient negative-strand RNA virus according to any one of the preceding items, wherein an endogenous sequence encoding the F polypeptide and/or an endogenous sequence encoding the HN polypeptide is modified, such that their function or activity is at least partially or completely disrupted, and the nucleotide sequence (b) is inserted in the locus of the F and/or HN gene.
23. The recombinant replication-deficient negative-strand RNA virus according to item 22, wherein an endogenous sequence encoding the HN polypeptide is modified, such its their function or activity is at least partially or completely disrupted, and the nucleotide sequence (b) is inserted in the locus of the HN gene.
24. The recombinant replication-deficient negative-strand RNA virus according to item 22, wherein an endogenous sequence encoding the F polypeptide is modified, such its their function or activity is at least partially or completely disrupted, and the nucleotide sequence (b) is inserted in the locus of the F gene.
25. The recombinant replication-deficient negative-strand RNA virus according to item 22, wherein an endogenous sequence encoding the F polypeptide and an endogenous sequence encoding the HN polypeptide are modified, such that their function or activity is at least partially or completely disrupted, and the nucleotide sequence (b) is inserted in the locus of the F and HN gene.
26. The recombinant replication-deficient negative-strand RNA virus according to any one of the preceding items, being capable of adsorbing at and penetrating into a host cell when displayed on the surface of the virus.
27. The recombinant replication-deficient negative-strand RNA virus according to any one of the preceding items, wherein the heterologous polypeptide mediates adsorption at and penetration into a host cell when displayed on the surface of the virus.
28. The recombinant replication-deficient negative-strand RNA virus according to item 27, wherein the host cell is a eukaryotic cell.
29. The recombinant replication-deficient negative-strand RNA virus according to any one of the preceding items, which is capable of infecting a eukaryotic cell.
30. The recombinant replication-deficient negative-strand RNA virus according to any one of the preceding items, capable of being rescued from eukaryotic cell.
31. The recombinant replication-deficient negative-strand RNA virus according to any one of the preceding items, capable of being propagated in a eukaryotic cell, said cell being complemented in trans with a sequence deficient in the replication-deficient negative-strand RNA virus, in particular complemented in trans with a sequence encoding the P polypeptide.
32. The recombinant replication-deficient negative-strand RNA virus according to any one of the preceding items, capable of being propagated in a eukaryotic cell complemented in trans with a sequence encoding
   (i) the P polypeptide, and/or
   (ii) the N and L polypeptide.
33. The recombinant replication-deficient negative-strand RNA virus according to any one of the preceding items, wherein the heterologous polypeptide encoded by the at least one nucleotide sequence (b) comprises a fragment of the Spike polypeptide.
34. The recombinant replication-deficient negative-strand RNA virus according to any one of the preceding items, wherein the nucleotide sequence (b) encodes
   (i) the ectodomain of the SARS-COV-2 Spike polypeptide, or a fragment thereof,
   (ii) the transmembrane domain of the SeV F or HN polypeptide, or a fragment thereof, and
   (iii) the cytoplasmic domain of the SeV F or HN polypeptide, or a fragment thereof.
35. The recombinant replication-deficient negative-strand RNA virus according to any one of the preceding items, wherein the nucleotide sequence (b) encodes
   (i) the ectodomain of the SARS-COV-2 Spike polypeptide, or a fragment thereof,
   (ii) the transmembrane domain of the SARS-COV-2 Spike polypeptide, or a fragment thereof, and
   (iii) the cytoplasmic domain of the SeV F or HN polypeptide, or a fragment thereof.
36. The recombinant replication-deficient negative-strand RNA virus according to any one of the preceding items, wherein the nucleotide sequence (b) encodes
   (i) the ectodomain of the SARS-COV-2 Spike polypeptide, or a fragment thereof,
   (ii) the transmembrane domain of the SARS-COV-2 Spike polypeptide, or a fragment thereof, and
   (iii) the cytoplasmic domain of the SARS-COV-2 Spike polypeptide, or a fragment thereof.
37. The recombinant replication-deficient negative-strand RNA virus according to any one of the preceding items, having reduced anti-vector antigenicity.
38. The recombinant replication-deficient negative-strand RNA virus according to any one of the preceding items, wherein the ectodomain of the SARS-COV-2 Spike polypeptide comprises an S1 subunit comprising a receptor binding domain (RBD), capable of binding to a host cell ACE-2 receptor.
39. The recombinant replication-deficient negative-strand RNA virus according to any one of the preceding items, wherein the S1 subunit can be cleaved off by a host cell ACE-2 protease.
40. The recombinant replication-deficient negative-strand RNA virus according to any one of the preceding items, wherein the ectodomain of the SARS-COV-2 Spike polypeptide comprises an S2 subunit, capable of fusing the SeV envelope with the host cell membrane or endosome membrane.
41. The recombinant replication-deficient negative-strand RNA virus according to any one of the preceding items, wherein the sequence of the ectodomain comprises at least one sequence modification capable of stabilizing the ectodomain in the prefusion form.
42. The recombinant replication-deficient negative-strand RNA virus according to any one of the preceding items, capable of inducing a mucosal immune response against the at least one heterologous polypeptide encoded by the at least one nucleotide sequence (b).
43. The recombinant replication-deficient negative-strand RNA virus according to any one of the preceding items, capable of inducing a protective immunity response against a pathogen carrying a polypeptide antigen encoded by the at least one nucleotide sequence (b).
44. Recombinant replication-deficient negative-strand RNA virus particle, comprising a nucleocapsid and an envelope, said nucleocapsid comprising in its genome:
   (a) a nucleotide sequence encoding the SeV P polypeptide, said nucleotide sequence being modified in comparison to the wildtype form, said modification leading to replication deficiency of the negative-strand RNA virus, and
   (b) at least one nucleotide sequence encoding at least one heterologous polypeptide, wherein the at least one heterologous polypeptide is displayed on the surface of the virus particle.
45. The recombinant replication-deficient negative-strand RNA virus particle according to item 44, wherein the at least one nucleotide sequence (b) encodes a SARS-COV-2 Spike polypeptide, or a fragment thereof, and said envelope comprises the SARS-COV-2 Spike polypeptide, or fragment thereof being displayed on the surface of the Sendai virus particle.
46. A recombinant replication-deficient Sendai virus, comprising in its genome:
   (a) a nucleotide sequence encoding a P polypeptide, said nucleotide sequence being modified in comparison to the wildtype form, said modification leading to replication deficiency of the negative-strand RNA virus, and
   (b) at least one nucleotide sequence encoding at least one heterologous polypeptide, wherein the at least one heterologous polypeptide is displayed on the surface of the virus particle.
47. The recombinant replication-deficient Sendai virus (SeV) according to item 46, wherein the at least one nucleotide sequence (b) encodes a SARS-COV-2 Spike polypeptide, or a fragment thereof, and said envelope comprises the SARS-COV-2 Spike polypeptide, or fragment thereof being displayed on the surface of the Sendai virus particle.
48. Recombinant replication-deficient Sendai virus particle, comprising a nucleocapsid and an envelope, said nucleocapsid comprising in its genome:
   (a) a nucleotide sequence encoding the SeV P polypeptide, said nucleotide sequence being modified in comparison to the wildtype form, said modification leading to replication deficiency of the negative-strand RNA virus, and
   (b) at least one nucleotide sequence encoding at least one heterologous polypeptide, wherein the at least one heterologous polypeptide is displayed on the surface of the Sendai virus particle.
49. The recombinant replication-deficient Sendai virus (SeV) particle according to item 48, wherein the at least one nucleotide sequence (b) encodes a SARS-COV-2 Spike polypeptide, or a fragment thereof, and said envelope comprises the SARS-COV-2 Spike polypeptide, or fragment thereof being displayed on the surface of the Sendai virus particle.
50. Pharmaceutical composition comprising the recombinant replication-deficient negative-strand RNA virus according to any one of the items 1-43 and/or the recombinant replication-deficient negative-strand RNA virus particle of any one of the items 44-49.
51. Vaccine, comprising the recombinant replication-deficient negative-strand RNA virus according to any one of the items 1-43 and/or the recombinant replication-deficient negative-strand RNA virus particle of any one of the items 44-49.
52. Immunogenic composition, comprising the recombinant replication-deficient negative-strand RNA virus according to any one of the items 1-43 and/or the recombinant replication-deficient negative-strand RNA virus particle of any one of the items 44-49.
53. The recombinant replication-deficient negative-strand RNA virus according to any one of the items 1-43, the recombinant replication-deficient negative-strand RNA virus particle of any one of the items 44-49, the pharmaceutical composition according to item 50, the vaccine according to item 51, or the immunogenic composition according to item 52, for medical use.
54. The recombinant replication-deficient negative-strand RNA virus according to any one of the items 1-43, the recombinant replication-deficient negative-strand RNA virus particle of any one of the items 44-49, the pharmaceutical composition according to item 50, the vaccine according to item 51, or the immunogenic composition according to item 52, for use in a method for prevention of a SARS-COV-2 infection.
55. The recombinant replication-deficient negative-strand RNA virus according to any one of the items 1-43, the recombinant replication-deficient negative-strand RNA virus particle of any one of the items 44-49, the pharmaceutical composition according to item 50, the vaccine according to item 51, or the immunogenic composition according to item 52, for use in a method for prevention of COVID 19.
56. The recombinant replication-deficient negative-strand RNA virus according to any one of the items 1-43, the recombinant replication-deficient negative-strand RNA virus particle of any one of the items 44-49, the pharmaceutical composition according to item 50, the vaccine according to item 51, or the immunogenic composition according to item 52, for use as a vaccine.
57. The recombinant replication-deficient negative-strand RNA virus according to any one of the items 1-43, the recombinant replication-deficient negative-strand RNA virus particle of any one of the items 44-49, the pharmaceutical composition according to item 50, the vaccine according to item 51, or the immunogenic composition according to item 52 for use of any one of the items 53-56, administered by the intranasal and/or mucosal route.
58. RNA molecule encoding a recombinant replication-deficient negative-strand RNA virus, said RNA molecule comprising
   (a) a nucleotide sequence encoding a P polypeptide, said nucleotide sequence being modified in comparison to the wildtype form, and
   (b) at least one nucleotide sequence encoding at least one heterologous polypeptide.
59. The RNA molecule according to item 58, further comprising
   (c) a nucleotide sequence encoding a N polypeptide,
   (d) a nucleotide sequence encoding a M polypeptide, and
   (e) a nucleotide sequence encoding a L polypeptide.
60. Nucleocapsid, comprising the RNA molecule according to item 58 or 59.
61. DNA molecule, encoding the RNA molecule according to item 58 or 59.
62. Host cell, comprising the replication-deficient negative-strand RNA virus of any one of the items 1-43, the recombinant replication-deficient negative-strand RNA virus particle of any one of the items 44-49, the RNA molecule according to item 58 or 59, the nucleocapsid according to item 60 and/or DNA molecule according to item 61.
63. Method for prevention of an infection with SARS-COV-2 and/or COVID 19, comprising administering to a subject in need thereof the recombinant replication-deficient negative-strand RNA virus according to any one of the items 1-43, the recombinant replication-deficient negative-strand RNA virus particle of any one of the items 44-49, the pharmaceutical composition according to item 50, the vaccine according to item 51, or the immunogenic composition according to item 52.
64. Method for production of the pharmaceutical composition according to item 50, the vaccine according to item 51, or the immunogenic composition according to item 52, comprising formulating the recombinant replication-deficient negative-strand RNA virus according to any one of the items 1-43 or the recombinant replication-deficient negative-strand RNA virus particle according to any one of the items 44-49, together with at least one pharmaceutically acceptable excipient.
65. Method for production of the recombinant negative-strand RNA virus or virus particle according to any one of the items 1-45, comprising
   (a) transfecting a eukaryotic host cell with a DNA molecule according to item 61, said host cell being capable of expressing the SeV polypeptides N, P and L,
   (b) cultivating the host cell under conditions such that said DNA molecule is transcribed, and the virus particle is formed, and
   (c) isolating the virus particle of (b).
66. Method for production of the recombinant negative-strand RNA virus or virus particle according to any one of the items 1-45, comprising
   (a) infecting a eukaryotic host cell with a wildtype negative strand RNA virus, and transfecting the eukaryotic host cell with a DNA molecule according to item 61, encoding a recombinant replication-deficient RNA virus, and
   (b) cultivating the host cell under conditions such that said DNA molecule is transcribed, and the virus particle is formed, and
   (c) isolating the virus particle of (b).
67. The method of item 66, wherein the wildtype negative strand RNA virus and the recombinant replication-deficient RNA virus are derived from the same species.
68. Use of the host cell of item 62 for the production of the recombinant replication-deficient negative-strand RNA virus according to any one of the items 1-43, and/or the recombinant replication-deficient negative-strand RNA virus particle of any one of the claims 44-49.
69. Use of a recombinant replication-deficient negative-strand RNA virus according to any one of the items 1-43, for the manufacture of a medicament for the prevention of an infection with SARS-CoV-2 and/or COVID 19.
70. A recombinant replication-competent negative-strand RNA virus, comprising in its genome:
   (a) a nucleotide sequence encoding a P polypeptide, wherein the P protein exerts in particular its natural function in the viral replication cycle,
   (b) at least one nucleotide sequence encoding at least one heterologous polypeptide, wherein the at least one heterologous polypeptide is displayed on the surface of the virus particle,
wherein the heterologous polypeptide comprises a viral antigen, and wherein the viral antigen and the negative-stranded RNA virus are selected from viruses of different families.
71. The recombinant replication-competent negative-strand RNA virus according to item 68, which is attenuated.
72. The recombinant replication-deficient negative-strand RNA virus according to any one of the items 70-71, which is a Paramyxovirus, Artovirus, Bornavirus, Filovirus, Lispivirus, Mymonavirus, Nyamivirus, Pneumovirus, Rhabdovirus, Sunvirus, or Xinmovirus.
73. The recombinant replication-competent negative-strand RNA virus according to any one of the items 70-72, which is a Sendai virus.
74. The recombinant replication-competent negative-strand RNA virus according to any one of the items 70-73, wherein the genome further comprises
   (c) a nucleotide sequence encoding a negative-strand RNA virus N polypeptide,
   (d) a nucleotide sequence encoding a negative-strand RNA virus M polypeptide, and
   (e) a nucleotide sequence encoding a negative-strand RNA virus L polypeptide,
75. The recombinant replication-competent negative-strand RNA virus according to any one of the items 70-74, wherein the heterologous polypeptide comprises a heterologous antigen.
76. The recombinant replication-competent negative-strand RNA virus according to any one of the items 70-75, wherein the heterologous polypeptide comprises a viral antigen.
77. The recombinant replication-competent negative-strand RNA virus according to item 76, wherein the viral antigen and the negative-stranded RNA virus are selected from viruses of different families.
78. The recombinant replication-competent negative-strand RNA virus according to any one of the items 70-77, wherein the heterologous polypeptide comprises an antigen from a virus which is not a negative-strand RNA virus.
79. The recombinant replication-competent negative-strand RNA virus according to any one of the items 70-78, wherein the heterologous polypeptide comprises an antigen from a positive-strand virus, such as a coronavirus.
80. The recombinant replication-competent negative-strand RNA virus according to any one of the items 70-79, wherein the negative-strand RNA virus is a paramyxovirus, such as a Sendai virus, and the heterologous polypeptide comprises an antigen from a coronavirus.
81. The recombinant replication-competent negative-strand RNA virus according to any one of the items 70-80, wherein the heterologous polypeptide comprises a coronavirus Spike protein (S protein), or an immunogenic fragment thereof.
82. The recombinant replication-competent negative-strand RNA virus according to any one of the items 70-81, wherein the coronavirus is SARS-COV-2.
83. The recombinant replication-competent negative-strand RNA virus according to any one of the items 70-82, wherein the at least one nucleotide sequence (b) is located in the RNA molecule between two coding sequences, at a position upstream the 5′ terminal coding sequence and/or downstream the 3′ terminal coding sequence.
84. The recombinant replication-competent negative-strand RNA virus according to any one of the items 70-83, wherein the genome further comprises
   (f) a nucleotide sequence encoding a negative-strand RNA virus F polypeptide, and
   (g) a nucleotide sequence encoding a negative-strand RNA virus HN polypeptide.
85. The recombinant replication-competent negative-strand RNA virus according to any one of the items 70-84, wherein the at least one nucleotide sequence (b) is inserted in or replaces at least partially at least one native (endogenous) sequence encoding at least one viral polypeptide, such that the function or activity of the at least one endogenous viral polypeptide is disrupted at least partially or completely.
86. The recombinant replication-competent negative-strand RNA virus according to any one of the items 70-85, wherein the nucleotide sequence (b) encoding the at least one heterologous polypeptide is essential to viral replication and/or propagation.
87. The recombinant replication-competent negative-strand RNA virus according to any one of the items 70-86, wherein the heterologous polypeptide encoded by sequence (b) is functionally expressed (in biologically active form).
88. The recombinant replication-competent negative-strand RNA virus according to any one of the items 70-87, wherein an endogenous sequence encoding the F polypeptide and/or an endogenous sequence encoding the HN polypeptide is modified, such that their function or activity is at least partially or completely disrupted, and the nucleotide sequence (b) is inserted in the locus of the F and/or HN gene.
89. The recombinant replication-competent negative-strand RNA virus according to item 88, wherein an endogenous sequence encoding the HN polypeptide is modified, such its their function or activity is at least partially or completely disrupted, and the nucleotide sequence (b) is inserted in the locus of the HN gene.
90. The recombinant replication-competent negative-strand RNA virus according to item 88, wherein an endogenous sequence encoding the F polypeptide is modified, such its their function or activity is at least partially or completely disrupted, and the nucleotide sequence is inserted in the locus of the F gene.
91. The recombinant replication-competent negative-strand RNA virus according to item 88, wherein an endogenous sequence encoding the F polypeptide and an endogenous sequence encoding the HN polypeptide are modified, such that their function or activity is at least partially or completely disrupted, and the nucleotide sequence is inserted in the locus of the F and HN gene.
92. The recombinant replication-competent negative-strand RNA virus according to any one of the items 70-91, being capable of adsorbing at and penetrating into a host cell when displayed on the surface of the virus.
93. The recombinant replication-competent negative-strand RNA virus according to any one of the items 70-92, wherein the heterologous polypeptide mediates adsorption at and penetration into a host cell when displayed on the surface of the virus.
94. The recombinant replication-competent negative-strand RNA virus according to item 93, wherein the host cell is a eukaryotic cell.
95. The recombinant replication-competent negative-strand RNA virus according to any one of the items 70-94, which is capable of infecting a eukaryotic cell.
96. The recombinant replication-competent negative-strand RNA virus according to any one of the items 70-95, capable of being rescued from eukaryotic cell.
97. The recombinant replication-competent negative-strand RNA virus according to any one of the items 70-96, capable of being propagated in a eukaryotic cell, said cell being complemented in trans with a sequence deficient in the replication-competent negative-strand RNA virus, in particular complemented in trans with a sequence encoding the P polypeptide.
98. The recombinant replication-competent negative-strand RNA virus according to any one of the items 70-97, wherein the heterologous polypeptide encoded by the at least one nucleotide sequence (b) comprises a fragment of the Spike polypeptide.
99. The recombinant replication-competent negative-strand RNA virus according to any one of the items 70-98, wherein the nucleotide sequence (b) encodes
   (i) the ectodomain of the SARS-COV-2 Spike polypeptide, or a fragment thereof,
   (ii) the transmembrane domain of the SeV F or HN polypeptide, or a fragment thereof, and
   (iii) the cytoplasmic domain of the SeV F or HN polypeptide, or a fragment thereof.
100. The recombinant replication-competent negative-strand RNA virus according to any one of the items 70-99, wherein the nucleotide sequence (b) encodes
   (i) the ectodomain of the SARS-COV-2 Spike polypeptide, or a fragment thereof,
   (ii) the transmembrane domain of the SARS-COV-2 Spike polypeptide, or a fragment thereof, and
   (iii) the cytoplasmic domain of the SeV F or HN polypeptide, or a fragment thereof.
101. The recombinant replication-competent negative-strand RNA virus according to any one of the items 70-100, wherein the nucleotide sequence (b) encodes
   (i) the ectodomain of the SARS-COV-2 Spike polypeptide, or a fragment thereof,
   (ii) the transmembrane domain of the SARS-COV-2 Spike polypeptide, or a fragment thereof, and
   (iii) the cytoplasmic domain of the SARS-COV-2 Spike polypeptide, or a fragment thereof.
102. The recombinant replication-competent negative-strand RNA virus according to any one of the items 70-101, having reduced anti-vector antigenicity.
103. The recombinant replication-competent negative-strand RNA virus according to any one of the items 70-102, wherein the ectodomain of the SARS-COV-2 Spike polypeptide comprises an S1 subunit comprising a receptor binding domain (RBD), capable of binding to a host cell ACE-2 receptor.
104. The recombinant replication-competent negative-strand RNA virus according to any one of the items 70-103, wherein the S1 subunit can be cleaved off by a host cell ACE-2 protease.
105. The recombinant replication-competent negative-strand RNA virus according to any one of the items 70-104, wherein the ectodomain of the SARS-COV-2 Spike polypeptide comprises an S2 subunit, capable of fusing the SeV envelope with the host cell membrane or endosome membrane.
106. The recombinant replication-competent negative-strand RNA virus according to any one of the items 70-105, wherein the sequence of the ectodomain comprises at least one sequence modification capable of stabilizing the ectodomain in the prefusion form.
107. The recombinant replication-competent negative-strand RNA virus according to any one of the items 70-106, capable of inducing a mucosal immune response against the at least one heterologous polypeptide encoded by the at least one nucleotide sequence (b).
108. The recombinant replication-competent negative-strand RNA virus according to any one of the items 70-107, capable of inducing a protective immunity response against a pathogen carrying a polypeptide antigen encoded by the at least one nucleotide sequence (b).
109. Recombinant replication-competent negative-strand RNA virus particle, comprising a nucleocapsid and an envelope, said nucleocapsid comprising in its genome:
   (a) a nucleotide sequence encoding a P polypeptide, wherein the P protein exerts in particular its natural function in the viral replication cycle,
   (b) at least one nucleotide sequence encoding at least one heterologous polypeptide, wherein the at least one heterologous polypeptide is displayed on the surface of the virus particle,
110. The recombinant replication-competent negative-strand RNA virus particle according to item 109, wherein the at least one nucleotide sequence (b) encodes a SARS-COV-2 Spike polypeptide, or a fragment thereof, and said envelope comprises the SARS-COV-2 Spike polypeptide, or fragment thereof being displayed on the surface of the Sendai virus particle.
111. A recombinant replication-competent Sendai virus, comprising in its genome:
   (a) a nucleotide sequence encoding a P polypeptide, wherein the P protein exerts in particular its natural function in the viral replication cycle,
   (b) at least one nucleotide sequence encoding at least one heterologous polypeptide, wherein the at least one heterologous polypeptide is displayed on the surface of the virus particle.
112. The recombinant replication-competent Sendai virus (SeV) according to item 111, wherein the at least one nucleotide sequence (b) encodes a SARS-COV-2 Spike polypeptide, or a fragment thereof, and said envelope comprises the SARS-COV-2 Spike polypeptide, or fragment thereof being displayed on the surface of the Sendai virus particle.
113. Recombinant replication-competent Sendai virus particle, comprising a nucleocapsid and an envelope, said nucleocapsid comprising in its genome:
   (a) a nucleotide sequence encoding a P polypeptide, wherein the P protein exerts in particular its natural function in the viral replication cycle,
   (b) at least one nucleotide sequence encoding at least one heterologous polypeptide, wherein the at least one heterologous polypeptide is displayed on the surface of the Sendai virus particle.
114. The recombinant replication-competent Sendai virus (SeV) particle according to item 113, wherein the at least one nucleotide sequence (b) encodes a SARS-COV-2 Spike polypeptide, or a fragment thereof, and said envelope comprises the SARS-COV-2 Spike polypeptide, or fragment thereof being displayed on the surface of the Sendai virus particle.
115. The recombinant replication-competent Sendai virus or particle according to any one of the item 111-114, which is attenuated.
116. Pharmaceutical composition comprising the recombinant replication-competent negative-strand RNA virus or virus particle according to any one of the items 70-110, or the recombinant replication-competent Sendai virus or virus particle according to any one of the items 111-115.
117. Vaccine, comprising the recombinant replication-competent negative-strand RNA virus or virus particle according to any one of the items 70-110, or the recombinant replication-competent Sendai virus or virus particle according to any one of the items 111-115.
118. Immunogenic composition, comprising the recombinant replication-competent negative-strand RNA virus or virus particle according to any one of the items 70-110, or the recombinant replication-competent Sendai virus or virus particle according to any one of the items 111-115.
119. The recombinant replication-competent negative-strand RNA virus or virus particle according to any one of the items 70-110, the recombinant replication-competent Sendai virus or virus particle according to any one of the items 111-115, the pharmaceutical composition according to item 116, the vaccine according to item 117, or the immunogenic composition according to item 118, for medical use.
120. The recombinant replication-competent negative-strand RNA virus or virus particle according to any one of the items 70-110, the recombinant replication-competent Sendai virus or virus particle according to any one of the items 111-115, the pharmaceutical composition according to item 116, the vaccine according to item 117, or the immunogenic composition according to item 118, for use in a method for prevention of a SARS-COV-2 infection.
121. The recombinant replication-competent negative-strand RNA virus or virus particle according to any one of the items 70-110, the recombinant replication-competent Sendai virus or virus particle according to any one of the items 111-115, the pharmaceutical composition according to item 116, the vaccine according to item 117, or the immunogenic composition according to item 118, for use in a method for prevention of COVID 19.
122. The recombinant replication-competent negative-strand RNA virus or virus particle according to any one of the items 70-110, the recombinant replication-competent Sendai virus or virus particle according to any one of the items 111-115, the pharmaceutical composition according to item 116, the vaccine according to item 117, or the immunogenic composition according to item 118, for use as a vaccine.
123. The recombinant replication-competent negative-strand RNA virus or virus particle according to any one of the items 70-110, the recombinant replication-competent Sendai virus or virus particle according to any one of the items 111-115, the pharmaceutical composition according to item 116, the vaccine according to item 117, or the immunogenic composition according to item 118, for use of any one of the items 119-122, administered by the intranasal and/or mucosal route.
124. RNA molecule encoding a recombinant replication-competent negative-strand RNA virus, said RNA molecule comprising
   (a) a nucleotide sequence encoding a P polypeptide, wherein the P protein exerts in particular its natural function in the viral replication cycle, and
   (b) at least one nucleotide sequence encoding at least one heterologous polypeptide.
125. The RNA molecule according to item 124, further comprising
   (a) a nucleotide sequence encoding a N polypeptide,
   (b) a nucleotide sequence encoding a M polypeptide, and
   (c) a nucleotide sequence encoding a L polypeptide.
126. Nucleocapsid, comprising the RNA molecule according to any one of the items 124-125.
127. DNA molecule, encoding the RNA molecule according to any one of the items 124-125.
128. Host cell, comprising the recombinant replication-competent negative-strand RNA virus or virus particle according to any one of the items 70-110, the recombinant replication-competent Sendai virus or virus particle according to any one of the items 111-115, the RNA molecule according to any one of the items 124-125, the nucleocapsid according to item 126 and/or DNA molecule according to item 127.
129. Method for prevention of an infection with SARS-COV-2 and/or COVID 19, comprising administering to a subject in need thereof the recombinant replication-competent negative-strand RNA virus or virus particle according to any one of the items 70-110, the recombinant replication-competent Sendai virus or virus particle according to any one of the items 111-115, the pharmaceutical composition according to item 116, the vaccine according to item 117, or the immunogenic composition according to item 118.
130. Method for production of the pharmaceutical composition according to item 116, the vaccine according to item 117, or the immunogenic composition according to item 118, comprising formulating the recombinant replication-competent negative-strand RNA virus or virus particle according to any one of the items 70-110, or the recombinant replication-competent Sendai virus or virus particle according to any one of the items 111-115, together with at least one pharmaceutically acceptable excipient.
131. Method for production of the recombinant replication-competent negative-strand RNA virus or virus particle according to any one of the items 70-110, or the recombinant replication-competent Sendai virus or virus particle according to any one of the items 111-115, comprising
   (a) transfecting a eukaryotic host cell with a DNA molecule according to item 127,
   (b) cultivating the host cell under conditions such that said DNA molecule is transcribed, and the virus particle is formed, and
   (c) isolating the virus particle of (b).
132. Use of the host cell of item 128 for the production of recombinant replication-competent negative-strand RNA virus or virus particle according to any one of the items 70-110, or the recombinant replication-competent Sendai virus or virus particle according to any one of the items 111-115.
133. Use of a recombinant replication-competent negative-strand RNA virus or virus particle according to any one of the items 70-110, or the recombinant replication-competent Sendai virus or virus particle according to any one of the items 111-115, for the manufacture of a medicament for the prevention of an infection with SARS-COV-2 and/or COVID 19.

1. A negative-strand RNA virus, comprising in its genome:
(a) a nucleotide sequence encoding a P polypeptide, wherein
(i) the P polypeptide is modified in comparison to a respective wildtype P polypeptide, said modification providing a replication-deficient negative-strand RNA virus, or
(ii) the P polypeptide has the same function in the viral replication cycle as a respective wildtype P polypeptide, or is a wildtype P polypeptide with the sequence SEQ ID NO: 002, providing a replication-competent negative-strand RNA virus; and
(b) at least one nucleotide sequence encoding at least one heterologous polypeptide, wherein
the at least one heterologous polypeptide is displayed on the surface of the virus particle, wherein the heterologous polypeptide comprises a viral antigen, and wherein the viral antigen and the negative-stranded RNA virus are selected from viruses of different families.

2. The recombinant replication-deficient negative-strand RNA virus according to claim 1, wherein the modification in the nucleotide sequence (a)i) is
(i) a deletion of the nucleotide sequence encoding the amino acids 2-77 of the respective wildtype P polypeptide, or
(ii) a deletion, insertion or substitution of the nucleotide sequence encoding amino acids 33-41 of the respective wildtype P polypeptide.

3. (canceled)

4. The recombinant replication-competent negative-stranded RNA virus according to claim 1(a)ii), which is attenuated.

5. The recombinant replication-deficient or replication-competent negative-strand RNA virus according to claim 1, wherein no fusion (F) polypeptide, and/or hemagglutinin (HN) polypeptide is displayed on the surface of the virus particle.

6. The recombinant replication-deficient or replication-competent negative-strand RNA virus according to claim 1, which is a Sendai virus.

7. The recombinant replication-deficient or replication-competent negative-strand RNA virus according to claim 1, which is transcription-competent.

8. The recombinant replication-deficient or replication-competent negative-strand RNA virus according to claim 1, wherein the heterologous polypeptide comprises a coronavirus Spike protein (S protein), or an immunogenic fragment thereof.

9. The recombinant replication-deficient or replication-competent negative-strand RNA virus according to claim 1, wherein an endogenous sequence encoding the F polypeptide and/or an endogenous sequence encoding the HN polypeptide is modified, such that their function or activity is at least partially or completely disrupted, and the nucleotide sequence (b) is inserted in the locus of the F and/or HN gene.

10. The recombinant replication-deficient or replication-competent negative-strand RNA virus according to claim 1, being capable of adsorbing at and penetrating into a host cell when displayed on the surface of the virus.

11. The recombinant replication-deficient or replication-competent negative-strand RNA virus according to claim 1, wherein the nucleotide sequence (b) encodes
A. (i) the ectodomain of the SARS-COV-2 Spike polypeptide, or a fragment thereof,
(ii) the transmembrane domain of the SeV F or HN polypeptide, or a fragment thereof, and
(iii) the cytoplasmic domain of the SeV F or HN polypeptide, or a fragment thereof, or
B. (i) the ectodomain of the SARS-COV-2 Spike polypeptide, or a fragment thereof,
(ii) the transmembrane domain of the SARS-COV-2 Spike polypeptide, or a fragment thereof, and
(iii) the cytoplasmic domain of the SeV F or HN polypeptide, or a fragment thereof, or
C. (i) the ectodomain of the SARS-COV-2 Spike polypeptide, or a fragment thereof,
(ii) the transmembrane domain of the SARS-COV-2 Spike polypeptide, or a fragment thereof, and
(iii) the cytoplasmic domain of the SARS-COV-2 Spike polypeptide, or a fragment thereof.

12. The recombinant replication-deficient or replication-competent negative-strand RNA virus according claim 1, capable of inducing a mucosal immune response against the at least one heterologous polypeptide encoded by the at least one nucleotide sequence (b).

13. Pharmaceutical composition comprising the recombinant replication-deficient or replication-competent negative-strand RNA virus according to claim 1.

14. A method for prevention of a SARS-COV-2 infection and/or COVID 19, comprising administering to a subject in need thereof the recombinant replication-deficient or replication-competent negative-strand RNA virus according to claim 1.

15. The recombinant replication-deficient or replication-competent negative-strand RNA virus according to claim 1, administered by the intranasal and/or mucosal route.

16. An RNA molecule or DNA molecule encoding a recombinant replication-deficient or replication-competent negative-strand RNA virus of claim 1.

17. A nucleocapsid, comprising the RNA molecule according to claim 16.

18. A host cell, comprising the replication-deficient or replication-competent negative-strand RNA virus of claim 1.

19. A method for prevention of a SARS-COV-2 infection and/or COVID 19, comprising administering to a subject in need thereof the pharmaceutical composition according to claim 13.

20. The pharmaceutical composition according to claim 13 administered by the intranasal and/or mucosal route.

21. The method of claim 14, administered by the intranasal and/or mucosal route.
